(19) United States
(12) Patent Application Publication
(10) Pub. No.: US 2006/0252107 A1
(43) Pub. Date: Nov. 9, 2006

(54) Compositions and methods for diagnosing and treating retinal diseases

(75) Inventors: Ryo Kubota, Seattle, WA (US); Krzysztof Palczewski, (US); Anna Gall, Woodinville, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Acucela, Inc., Seattle, WA (US)

(21) Appl. No.: 11/361,027

(22) Filed: Feb. 22, 2006

Related U.S. Application Data
(60) Provisional application No. 60/655,344, filed on Feb. 22, 2005.

(51) Int. Cl. G01N 33/53; C07K 16/18; C07K 14/47; C07D 213/55; C07D 213/20; C12N 5/06

(52) U.S. Cl. 435/7.92; 530/387.1; 530/409; 435/326; 546/341; 546/347

(57) ABSTRACT

The present invention relates generally to the preparation and use of retinoid N-retinylidene-N-retinyl-ethanolamine (A2E) conjugated to a carrier polypeptide and to the preparation and use of antibodies that bind specifically to A2E. The invention relates to the use of A2E conjugates as immunogens or vaccines and to the use of A2E specific antibodies for treatment of ophthalmic diseases. Provided herein are methods for enhancing retinal neuronal cell survival, including photoreceptor cell survival, using antibodies that specifically bind to A2E or by inducing an immune response using an A2E immunoconjugate. Enhancing survival of photoreceptor cells or decreasing accumulation of A2E in the eye using the A2E immunoconjugates or A2E specific antibodies is useful for treatment of ophthalmic diseases such as macular degeneration.

### CROSS-REFERENCE TO RELATED APPLICATION

This patent application claims the benefit of U.S. Provisional Patent Application No. 60/655,344 filed Feb. 22, 2005, which is incorporated herein by reference in its entirety.

### BACKGROUND OF THE INVENTION

1. Field of the Invention

The present invention relates generally to the preparation of retinoid N-retinylidene-N-retinyl-ethanolamine (A2E) derivatives and A2E-like compounds that may be conjugated to a carrier polypeptide for use as immunoconjugates and to the preparation and use of antibodies that bind specifically to A2E that are useful for treating and preventing ophthalmic diseases, such as macular degeneration. The antibodies that bind specifically to A2E are also useful for detecting A2E in a biological sample and for diagnosis and prognosis of an ophthalmic disease.

2. Description of the Related Art

Neurodegenerative diseases, such as glaucoma, macular degeneration, and Alzheimer's disease, affect millions of patients throughout the world. Because the loss of quality of life associated with these diseases is considerable, drug research and development in this area is of great importance.

Macular degeneration is a disease that affects central vision. Macular degeneration affects between five and ten million patients in the United States, and it is the leading cause of blindness worldwide. Macular degeneration is a disease that causes the loss of photoreceptor cells in the central part of retina called the macula. Macular degeneration can be classified into two types: dry type and wet type. The dry form is more common than the wet, with about 90% of age-related macular degeneration (ARMD) patients diagnosed with the dry form. The wet form of the disease usually leads to more serious vision loss. The exact causes of age-related macular degeneration are still unknown. The dry form of ARMD may result from the aging and thinning of macular tissues and from deposition of pigment in the macula. In wet ARMD, new blood vessels grow beneath the retina and leak blood and fluid. This leakage causes the retinal cells to die, creating blind spots in central vision.

One protocol approved by the Food and Drug Administration (FDA) for treating ARMD is a photodynamic therapy that uses a special drug combined with laser photocoagulation. This treatment, however, can only be applied to half of patients newly diagnosed with wet form of ARMD. Very recently, the FDA approved Macugen® (pegaptanib sodium injection) for the treatment of neovascular (wet) ARMD). For the vast majority of patients who have the dry form of macular degeneration, no treatment is available. Because the dry form precedes development of the wet form of macular degeneration, intervention in disease progression of the dry form could benefit patients that presently have dry form and may delay or prevent development of the wet form.

Declining vision noticed by the patient or by an ophthalmologist during a routine eye exam may be the first indicator of macular degeneration. The formation of exudates, or “drusen,” from blood vessels in and under the macular is often the first physical sign that macular degeneration may develop. Symptoms include perceived distortion of straight lines and, in some cases, the center of vision appears more distorted than the rest of a scene; a dark, blurry area or “white-out” appears in the center of vision; and/or color perception changes or diminishes.

Different forms of macular degeneration may also occur in younger patients. Non-age related etiology may be linked to heredity, diabetes, nutritional deficits, head injury, infection, or other factors.

Glaucoma is a broad term used to describe a group of diseases that causes visual field loss, often without any other prevailing symptoms. The lack of symptoms often leads to a delayed diagnosis of glaucoma until the terminal stages of the disease. Prevalence of glaucoma is estimated to be three million in the United States, with about 120,000 cases of blindness attributable to the condition. The disease is also prevalent in Japan, which has four million reported cases. In other parts of the world, treatment is less accessible than in the United States and Japan, thus glaucoma ranks as a leading cause of blindness worldwide. Even if subjects afflicted with glaucoma do not become blind, their vision is often severely impaired.

The loss of peripheral vision is caused by the death of ganglion cells in the retina. Ganglion cells are a specific type of projection neuron that connects the eye to the brain. Glaucoma is often accompanied by an increase in intraocular pressure. Current treatment includes use of drugs that lower the intraocular pressure; however, lowering the intraocular pressure is often insufficient to completely stop disease progression. Ganglion cells are believed to be susceptible to pressure and may suffer permanent degeneration prior to the lowering of intraocular pressure. An increasing number of cases of normal tension glaucoma has been observed in which ganglion cells degenerate without an observed increase in the intraocular pressure. Because current glaucoma drugs only treat intraocular pressure, a need exists to identify new therapeutic agents that will prevent or reverse the degeneration of ganglion cells. Recent reports suggest that glaucoma is a neurodegenerative disease, similar to Alzheimer's disease and Parkinson's disease in the brain, except that it specifically affects retinal neurons. The retinal neurons of the eye originate from diencephalon neurons of the brain. Though retinal neurons are often mistakenly thought not to be part of the brain, retinal cells are key components of vision, interpreting the signals from the light sensing cells.

Alzheimer's disease (AD) is the most common form of dementia among the elderly. Dementia is a brain disorder that seriously affects a person's ability to carry out daily activities. Alzheimer's is a disease that affects four million people in the United States alone. It is characterized by a loss of nerve cells in areas of the brain that are vital to memory and other mental functions. Some drugs can prevent AD symptoms for a finite period of time, but no drugs are available that treat the disease or completely stop the progressive decline in mental function. Recent research suggests that glial cells that support the neurons or nerve cells may have defects in AD sufferers, but the cause of AD remains unknown. Individuals with AD seem to have a higher incidence of glaucoma and macular degeneration, indicating that similar pathogenesis may underlie these neurodegenerative diseases of the eye and brain. (See Giasson et al., Free Radic. Biol. Med. 32:1264-75 (2002); Johnson et al., Proc. Natl. Acad. Sci. USA 99:11830-35 (2002); Dentchev et al., Mol. Vis. 9:184-90 (2003)).

Neuronal cell death underlies the pathology of these diseases. Unfortunately, very few compositions and methods that enhance retinal neuronal cell survival, particularly photoreceptor cell survival, have been discovered. A need therefore exists to identify and develop compositions that that can be used for treatment and prophylaxis of retinal diseases and disorders.

### BRIEF SUMMARY OF THE INVENTION

Briefly stated, the present invention relates to compounds, compositions, and methods for treatment of one or more retinal diseases and disorders. In particular, the invention provides retinoid N-retinylidene-N-retinyl-ethanolamine- (A2E)-like molecules A2E derivatives, and A2E carrier polypeptide conjugates. The invention also provides methods for using the conjugates as immunogens. Also provided are antibodies that bind specifically to A2E and that are useful for treating and preventing ophthalmic diseases, such as macular degeneration. The invention also provides methods for using the antibodies that bind specifically to A2E for detecting A2E in a biological sample and for diagnosis and prognosis of an ophthalmic disease.

In one embodiment, a compound is provided that has the following structure 1:

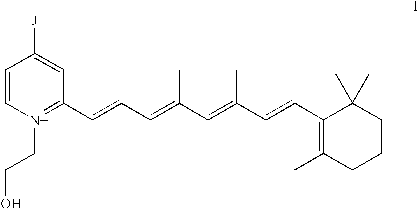

or a pharmaceutically acceptable acid addition salt thereof;

wherein

J is -Z¹-Y, -Z²-R⁰—Y, -Z²-R⁰-Z³-Y, or -Z²-Y′—R⁰,

Z¹ is a divalent C₁-C₄₀ alkyl,

Z² is a divalent C₁-C₄₀ alkyl,

Z³ is a polyethylene glycol having the formula —(CH₂CH₂O)ₙCH₂CH₂— wherein n=2-12, or —R⁴—CH₂—, wherein R⁴ is C₁-C₄₀ alkylene or C₁-C₄₀ heteroalkylene;

R⁰ is a monovalent or divalent optionally substituted homocycle, aryl, heteroaryl, or heterocycle; and

wherein Y is a monovalent electrophilic or nucleophilic moiety suitable for covalent attachment of the compound to an amino acid, and wherein Y′ is a divalent electrophilic or nucleophilic moiety suitable for covalent attachment of the compound to an amino acid. In certain embodiment, Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl. Additional embodiments of an A2E-like compound having structure 1 are described in detail herein.

In one embodiment, a compound is provided that has the following structure 2:

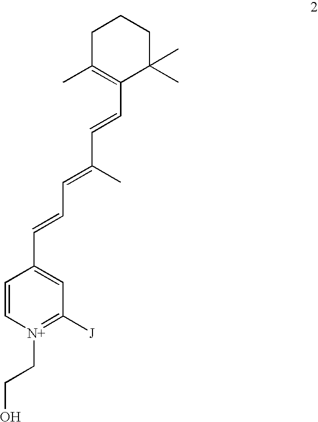

or a pharmaceutically acceptable acid addition salt thereof;

wherein

J is -Z¹-Y, -Z²-R⁰—Y, -Z²-R⁰-Z³-Y, or -Z²-Y′—R⁰,

Z¹ is a divalent C₁-C₄₀ alkyl,

Z² is a divalent C₁-C₄₀ alkyl,

Z³ is a polyethylene glycol having the formula —(CH₂CH₂O)ₙCH₂CH₂— wherein n=2-12, or —R⁴—CH₂—, wherein R⁴ is C₁-C₄₀ alkylene or C₁-C₄₀ heteroalkylene;

R⁰ is a monovalent or divalent optionally substituted homocycle, aryl, heteroaryl, or heterocycle; and

wherein Y is a monovalent electrophilic or nucleophilic moiety suitable for covalent attachment of the compound to an amino acid, and wherein Y′ is a divalent electrophilic or nucleophilic moiety suitable for covalent attachment of the compound to an amino acid. In a certain embodiment, Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl. Additional embodiments of an A2E-like compound having structure 2 are described in detail herein.

In another embodiment, a compound is provided that has the following structure 3:

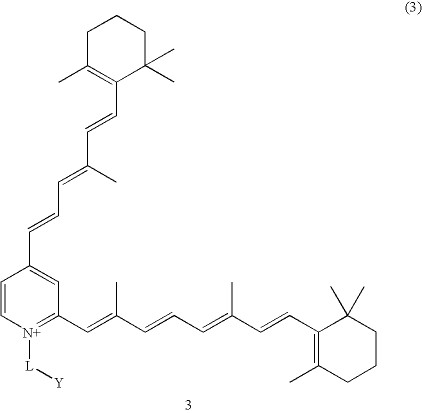

or a pharmaceutically acceptable acid addition salt thereof,

wherein L is a divalent linker group -R₁-, -R₂-, or -R₃-, and R₁ is divalent C₁-C₆ alkyl;

R₂ is C₁-C₄₀ alkylene or C₁-C₄₀ heteroalkylene; and

R₃ is a polyethylene glycol having the formula —(CH₂CH₂O)ₙCH₂CH₂— wherein n=2-12;

and wherein Y is an electrophilic or nucleophilic moiety suitable for reaction of the compound with an amino acid. In a certain embodiment, Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl. Additional embodiments of an A2E like compound having structure 3 are described in detail herein.

In another embodiment an immunogen is provided that comprises at least one molecule of any one of the aforementioned compounds described herein and any of the A2E like compounds described in detail herein that is conjugated, joined or attached or in some manner reacted with an amino acid of a hapten, such as a hapten peptide, or to a carrier polypeptide. In certain embodiments, the hapten peptide may be conjugated or attached or joined to a carrier polypeptide. In certain embodiments, the carrier polypeptide is keyhole limpet hemocyanin, bovine serum albumin, ovalbumin, tetanus toxoid, diphtheria toxoid, E. coli heat-labile enterotoxin B subunit, polyglutamate, glucose oxidase, rabbit serum albumin, sperm whale myoglobin, human thyroglobulin, or Pasteurella haemolytica leukotoxin polypeptide, or other carrier polypeptides used in the art. In a particular embodiment, the carrier polypeptide is keyhole limpet hemocyanin or bovine serum albumin, and in another particular embodiment the carrier polypeptide is tetanus toxoid, diphtheria toxoid, or E. coli heat-labile enterotoxin B subunit. Also provided herein is a composition that comprises the immunogen and a physiologically acceptable excipient. In a certain embodiment, the composition further comprises an adjuvant. In particular embodiments, the adjuvant is selected from alum, aluminum hydroxide, a CpG oligodeoxynucleotide, ISCOMATRIX® adjuvant, proteosomes, Protollin™, and monophosphoryl lipid A (MPL®). In another embodiment, a method is provided for producing an antibody that specifically binds to retinoid N-retinylidene-N-retinyl-ethanolamine (A2E) comprising (a) administering to a non-human animal the immunogen as described herein, and (b) isolating the antibody from the animal. In certain embodiments, the method further comprises administering an adjuvant in step (a), and in particular embodiments, the adjuvant is alum, aluminum hydroxide, a CpG oligodeoxynucleotide, ISCOMATRIX® adjuvant, proteosomes, Protollin™ or monophosphoryl lipid A (MPL®). The invention also provides a method for inducing an immune response in an animal comprising administering to the animal a composition that comprises the immunogen. In a particular embodiment, the composition further comprises an adjuvant. In certain specific embodiments, the adjuvant is alum, aluminum hydroxide, a CpG oligodeoxynucleotide, ISCOMATRIX® adjuvant, proteosomes, Protollin™, or monophosphoryl lipid A (MPL®).

Also provided is an isolated antibody, or an antigen-binding fragment thereof, that binds specifically to retinoid N-retinylidene-N-retinyl-ethanolamine (A2E). In one embodiment the antibody is a polyclonal antibody and in another embodiment, the antibody is a monoclonal antibody. In certain embodiments, the monoclonal antibody is a mouse monoclonal antibody, a human monoclonal antibody, a rat monoclonal antibody, or a hamster monoclonal antibody. In another embodiment the antibody is a chimeric antibody or a humanized antibody. In certain embodiments, the antigen-binding fragment of the antibody, including the monoclonal, polyclonal, humanized, or chimeric antibody, is an Fab, Fab′, F(ab′)₂, Fd, and Fv. In particular embodiments, the antigen-binding fragment is of human, mouse, chicken, or rabbit origin. In certain other embodiments, the antibody is a single chain antibody (scFv). In other embodiments, the antibody is a recombinant antibody. The invention also provides a host cell that expresses an antibody that binds specifically to retinoid N-retinylidene-N-retinyl-ethanolamine (A2E), and provides a host cell that expresses any one of the antibodies, or antigen-binding fragment thereof, described herein that specifically binds to A2E. The invention further provides a composition that comprises any one of the antibodies, or a binding fragment thereof, that binds specifically to A2E and a physiologically acceptable excipient.

In another embodiment, the invention provides a method for producing an antibody that specifically binds to retinoid N-retinylidene-N-retinyl-ethanolamine (A2E) comprising administering to an animal an immunogen that comprises a compound having any one of the structures described herein (e.g. structure 1, 2, 3, I, II, III, IV, V, VI, VII, and VIII, or substructure thereof as described in detail herein, and specific structures such as 1a, 1b, 1c, 2a, 2b, 3a, 3b 3c,) that is conjugated to a carrier polypeptide. In certain embodiments, the carrier polypeptide is keyhole limpet hemocyanin, bovine serum albumin, ovalbumin, tetanus toxoid, diphtheria toxoid, E. coli heat-labile enterotoxin B subunit, polyglutamate, glucose oxidase, rabbit serum albumin, sperm whale myoglobin, human thyroglobulin, or Pasteurella haemolytica leukotoxin polypeptide. In a particular embodiment, the carrier polypeptide is keyhole limpet hemocyanin or bovine serum albumin, and in another particular embodiment the carrier polypeptide is tetanus toxoid, diphtheria toxoid, or E. coli heat-labile enterotoxin B subunit. In certain specific embodiments, the method further comprises administering an adjuvant, wherein the adjuvant is alum, aluminum hydroxide, a CpG oligodeoxynucleotide, ISCOMATRIX® adjuvant, proteosomes, Protollin™, or monophosphoryl lipid A (MPL®).

In one embodiment is provided a method for detecting the presence of retinoid N-retinylidene-N-retinyl-ethanolamine (A2E) in a biological sample comprising: (a) contacting a biological sample with an antibody, or antigen-binding fragment thereof, that specifically binds to A2E, under conditions and for a time sufficient to permit formation of an antibody/A2E complex; and (b) detecting a level of antibody/A2E complex, and thereby detecting the presence of A2E in a sample. In certain embodiments, the biological sample is blood, serum, vitreous fluid, aqueous humor, intraocular fluid, or tears. In another certain embodiment, the antibody is detectably labeled. In a particular embodiment, the antibody is detectably labeled with a fluorophore, a radionuclide, an enzyme, or biotin.

In another embodiment, the invention provides a method for determining the presence of macular degeneration or determining the risk of developing macular degeneration in a subject comprising: (a) contacting a biological sample from a subject with an antibody that specifically binds to A2E under conditions and for a time sufficient to permit formation of an antibody/A2E complex; and (b) detecting a level of antibody/A2E complex, and thereby determining the presence of macular degeneration in a subject. In certain embodiments, the biological sample is blood, serum, vitreous fluid, aqueous humor, intraocular fluid, or tears. In another certain embodiment, the antibody is detectably labeled. In a particular embodiment, the antibody is detectably labeled with a fluorophore, a radionuclide, an enzyme, or biotin.

The invention also provides a method for inhibiting degeneration of a retinal cell or enhancing retinal cell survival comprising contacting a retinal cell with an antibody that specifically binds to retinoid N-retinylidene-N-retinyl-ethanolamine (A2E). In a specific embodiment, the retinal cell is a retinal neuronal cell. In another specific embodiment, the retinal neuronal cell is an amacrine cell, a horizontal cell, a bipolar cell, and ganglion cell, and a photoreceptor cell. In particular embodiments, the retinal neuronal cell is a photoreceptor cell.

In another embodiment, a method is provided for inhibiting degeneration of a retinal cell or enhancing retinal cell survival in a subject having an ophthalmic disease, comprising administering to the subject a composition comprising an antibody that specifically binds to retinoid N-retinylidene-N-retinyl-ethanolamine (A2E) and a physiologically acceptable excipient. In a specific embodiment, the retinal cell is a retinal neuronal cell. In another specific embodiment, the retinal neuronal cell is an amacrine cell, a horizontal cell, a bipolar cell, and ganglion cell, and a photoreceptor cell. In particular embodiments, the retinal neuronal cell is a photoreceptor cell. In other particular embodiments, the ophthalmic disease or disorder is macular degeneration, glaucoma, diabetic retinopathy, retinal detachment, retinal blood vessel occlusion, retinitis pigmentosa, optic neuropathy, inflammatory retinal disease, diabetic maculopathy, hemorrhagic retinopathy, retinopathy of prematurity, optic neuropathy, proliferative vitreoretinopathy, retinal dystrophy, ischemia-reperfusion related retinal injury, hereditary optic neuropathy, metabolic optic neuropathy, Stargardt's macular dystrophy, Sorsby's fundus dystrophy, Best disease, uveitis, a retinal injury, a retinal disorder associated with Parkinson's disease, a retinal disorder associated with viral infection, a retinal disorder related to light overexposure, and a retinal disorder associated with AIDS, a retinal disorder associated with Alzheimer's disease, and a retinal disorder associated with multiple sclerosis. In certain embodiments, the ophthalmic disease or disorder is macular degeneration or Stargardt's macular dystrophy, and in a specific embodiment macular degeneration is dry form macular degeneration. In other certain embodiments, ophthalmic disease or disorder is diabetic retinopathy or diabetic maculopathy, and in another certain embodiment, the ophthalmic disease or disorder is retinitis pigmentosa.

In still another embodiment, is provided a method for inhibiting accumulation of retinoid N-retinylidene-N-retinyl-ethanolamine (A2E) in an eye of a subject, comprising administering to the subject a composition comprising an antibody that specifically binds to retinoid N-retinylidene-N-retinyl-ethanolamine (A2E) and a physiologically acceptable excipient.

In another embodiment, the invention provides a method for treating an ophthalmic disease or disorder in a subject comprising administering to the subject a composition comprising an antibody that specifically binds to retinoid N-retinylidene-N-retinyl-ethanolamine (A2E) and a physiologically acceptable excipient. In particular embodiments, the ophthalmic disease or disorder is macular degeneration, glaucoma, diabetic retinopathy, retinal detachment, retinal blood vessel occlusion, retinitis pigmentosa, optic neuropathy, inflammatory retinal disease, diabetic maculopathy, hemorrhagic retinopathy, retinopathy of prematurity, optic neuropathy, proliferative vitreoretinopathy, retinal dystrophy, ischemia-reperfusion related retinal injury, hereditary optic neuropathy, metabolic optic neuropathy, Stargardt's macular dystrophy, Sorsby's fundus dystrophy, Best disease, uveitis, a retinal injury, a retinal disorder associated with Parkinson's disease, a retinal disorder associated with viral infection, a retinal disorder related to light overexposure, and a retinal disorder associated with AIDS, a retinal disorder associated with Alzheimer's disease, and a retinal disorder associated with multiple sclerosis. In certain embodiments, the ophthalmic disease or disorder is macular degeneration or Stargardt's macular dystrophy, and in a specific embodiment macular degeneration is dry form macular degeneration. In other certain embodiments, ophthalmic disease or disorder is diabetic retinopathy or diabetic maculopathy, and in another certain embodiment, the ophthalmic disease or disorder is retinitis pigmentosa.

In particular embodiments of the methods for inhibiting degeneration of a retinal cell or enhancing retinal cell survival, for inhibiting accumulation of retinoid N-retinylidene-N-retinyl-ethanolamine (A2E) in an eye of a subject, and for treating an ophthalmic disease in a subject, the antibody is a polyclonal antibody and in another embodiment, the antibody is a monoclonal antibody. In certain embodiments, the monoclonal antibody is a mouse monoclonal antibody, a human monoclonal antibody, a rat monoclonal antibody, or a hamster monoclonal antibody. In another embodiment the antibody is a chimeric antibody or a humanized antibody. In certain embodiments, the antigen-binding fragment of the antibody, including the monoclonal, polyclonal, humanized, or chimeric antibody, is an Fab, Fab′, F(ab′)₂, Fd, and Fv. In particular embodiments, the antigen-binding fragment is of human, mouse, chicken, or rabbit origin. In certain other embodiments, the antibody is a single chain antibody (scFv). In other embodiments, the antibody is a recombinant antibody.

Also provided herein are methods for treating an ophthalmic disease in a subject comprising administering to the subject an immunogen comprising any one of the aforementioned compounds described herein that is conjugated to a carrier polypeptide. In certain embodiments, the carrier polypeptide is keyhole limpet hemocyanin, bovine serum albumin, ovalbumin, tetanus toxoid, diphtheria toxoid, E. coli heat-labile enterotoxin B subunit, polyglutamate, glucose oxidase, rabbit serum albumin, sperm whale myoglobin, human thyroglobulin, or Pasteurella haemolytica leukotoxin polypeptide. In a particular embodiment, the carrier polypeptide is keyhole limpet hemocyanin or bovine serum albumin, and in another particular embodiment the carrier polypeptide is tetanus toxoid, diphtheria toxoid, or E. coli heat-labile enterotoxin B subunit. In another embodiment, the method further comprises administering an adjuvant. In specific embodiments, the adjuvant is alum, aluminum hydroxide, a CpG oligodeoxynucleotide, ISCOMATRIX® adjuvant, proteosomes, Protollin™, or monophosphoryl lipid A (MPL®).

In another embodiment, a method is provided for treating an ophthalmic disease or disorder comprising administering to a subject in need thereof the immunogen comprising any one of the aforementioned compounds described herein that is conjugated to a carrier polypeptide. In certain embodiments, the carrier polypeptide is keyhole limpet hemocyanin, bovine serum albumin, ovalbumin, tetanus toxoid, diphtheria toxoid, E. coli heat-labile enterotoxin B subunit, polyglutamate, glucose oxidase, rabbit serum albumin, sperm whale myoglobin, human thyroglobulin, or Pasteurella haemolytica leukotoxin polypeptide. In a particular embodiment, the carrier polypeptide is keyhole limpet hemocyanin or bovine serum albumin, and in another particular embodiment the carrier polypeptide is tetanus toxoid, diphtheria toxoid, or E. coli heat-labile enterotoxin B subunit. In another embodiment, the method further comprises administering an adjuvant. In specific embodiments, the adjuvant is alum, aluminum hydroxide, a CpG oligodeoxynucleotide, ISCOMATRIX® adjuvant, proteosomes, Protollin™, or monophosphoryl lipid A (MPL®). In particular embodiments, the ophthalmic disease or disorder is macular degeneration, glaucoma, diabetic retinopathy, retinal detachment, retinal blood vessel occlusion, retinitis pigmentosa, optic neuropathy, inflammatory retinal disease, diabetic maculopathy, hemorrhagic retinopathy, retinopathy of prematurity, optic neuropathy, proliferative vitreoretinopathy, retinal dystrophy, ischemia-reperfusion related retinal injury, hereditary optic neuropathy, metabolic optic neuropathy, Stargardt's macular dystrophy, Sorsby's fundus dystrophy, Best disease, uveitis, a retinal injury, a retinal disorder associated with Parkinson's disease, a retinal disorder associated with viral infection, a retinal disorder related to light overexposure, and a retinal disorder associated with AIDS, a retinal disorder associated with Alzheimer's disease, and a retinal disorder associated with multiple sclerosis. In certain embodiments, the ophthalmic disease or disorder is macular degeneration or Stargardt's macular dystrophy, and in a specific embodiment macular degeneration is dry form macular degeneration. In other certain embodiments, ophthalmic disease or disorder is diabetic retinopathy or diabetic maculopathy, and in another certain embodiment, the ophthalmic disease or disorder is retinitis pigmentosa.

These and other embodiments of the invention will become evident upon reference to the following detailed description and attached drawings. In addition, references set forth herein that describe in more detail certain embodiments of this invention are therefore incorporated by reference in their entireties. All U.S. patents, U.S. patent application publications, U.S. patent applications, foreign patents, foreign patent applications, and non-patent publications referred to in this specification and/or listed in the Application Data Sheet, are incorporated herein by reference, in their entireties.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells and equivalents thereof known to those skilled in the art, and so forth. The term “comprising” (and related terms such as “comprise” or “comprises” or “having” or “including” and the like) is not intended to exclude that in other certain embodiments, for example, an embodiment of any composition of matter, composition, method, or process, or the like, described herein may “consist of” or “consist essentially of” the described features.

### BRIEF DESCRIPTION OF THE DRAWINGS

FIG. 1 provides a schematic of a photoreceptor cell.

### DETAILED DESCRIPTION OF THE INVENTION

The present invention relates to the novel discovery that antibodies specific for retinoid N-retinylidene-N-retinyl-ethanolamine (A2E) enhance the survival or inhibit degeneration of retinal cells, particularly retinal neuronal cells (e.g., photoreceptor cells, amacrine cells, bipolar cells, horizontal cells, and ganglion cells). Also provided are novel A2E-like compounds, and A2E derivative compounds, and A2E-carrier immunoconjugates and methods for making same. The A2E-like compounds and A2E derivative compounds described herein may be used for conjugating (attaching) to a carrier molecule, such as a carrier polypeptide to form A2E-carrier polypeptide compounds. The A2E conjugates are useful for immunizing a host to induce an immune response and as such are useful for treating or preventing an ophthalmic disease or disorder, such as macular degeneration. The A2E conjugates described herein may also be used for immunizing a non-human animal for production and isolation of antibodies that specifically bind to A2E.

Neurodegeneration of stressed retinal cells, including retinal neuronal cells, particularly photoreceptor cells, is decreased in cells (that is, degeneration of the retinal cells is inhibited) that are concurrently or subsequently exposed to antibodies specific for A2E. Exposure of stressed retinal neuronal cells to antibodies that specifically bind to A2E results in an increased number of surviving photoreceptor cells. These antibodies may be used in methods for treating neurological diseases or disorders in general, and for treating degenerative diseases of the eye and brain in particular. The antibodies that specifically bind to A2E may be useful for treating, curing, preventing, ameliorating the symptoms of, or slowing, inhibiting, or stopping the progression of a neurodegenerative disease or disorder, particularly ophthalmic diseases or disorders. Representative ophthalmic diseases and disorders include but are not limited to macular degeneration, glaucoma, diabetic retinopathy, retinal detachment, retinal blood vessel occlusion, retinitis pigmentosa, optic neuropathy, inflammatory retinal disease, diabetic maculopathy, hemorrhagic retinopathy, retinopathy of prematurity, optic neuropathy, proliferative vitreoretinopathy, retinal dystrophy, ischemia-reperfusion related retinal injury, hereditary optic neuropathy, metabolic optic neuropathy, Stargardt's macular dystrophy, Sorsby's fundus dystrophy, Best disease, uveitis, a retinal injury, a retinal disorder associated with Parkinson's disease, a retinal disorder associated with viral infection, a retinal disorder related to light overexposure, and a retinal disorder associated with AIDS, a retinal disorder associated with Alzheimer's disease, and a retinal disorder associated with multiple sclerosis.

In another embodiment, an immune response, which may include a cellular response as well as a humoral response, may be induced using a composition (i.e., a vaccine) that comprises A2E conjugated to a carrier molecule such as a carrier polypeptide. Administration of an A2E conjugate (i.e., A2E immunoconjugates) as described herein may be used for treating a subject who has an ophthalmic disease or who is at risk for developing an ophthalmic disease. The immune response induced by an A2E immunoconjugate may be useful for treating, curing, preventing, ameliorating the symptoms of, or slowing, inhibiting, or stopping the progression of a neurodegenerative disease or disorder, particularly ophthalmic diseases or disorders by reducing, slowing, inhibiting, or stopping degeneration of retinal cells, for example, inhibiting neurodegeneration of retinal neuronal cells, particularly photoreceptor cells, that occurs as a result of exposure to A2E.

Accumulation of the pigment lipofuscin in retinal pigment epithelium (RPE) cells has been linked to progression of retinal diseases that result in blindness, including age-related macular degeneration (De Laey et al., Retina 15:399-406 (1995)). Lipofuscin granules are autofluorescent lysosomal residual bodies (also called age pigments). The major fluorescent species of lipofuscin is A2E (an orange-emitting fluorophore), which is a positively charged Schiff-base condensation-product formed by all-trans retinaldehyde with phosphatidylethanolamine (2:1 ratio) (see, e.g., Eldred et al., Nature 361:724-6 (1993); see also, Sparrow, Proc. Natl. Acad. Sci. USA 100:4353-54 (2003)). Much of the indigestible lipofuscin pigment is believed to originate in photoreceptor cells; deposition in the RPE occurs because the RPE internalize membranous debris that is discarded daily by the photoreceptor cells. Formation of this compound is not believed to occur by catalysis by any enzyme, but rather A2E forms by a spontaneous cyclization reaction. In addition, A2E has a pyridinium bisretinoid structure that once formed cannot be enzymatically degraded. Lipofuscin, and thus A2E, accumulate with aging of the human eye and also accumulate in a juvenile form of macular degeneration called Stargardt's disease.

A2E may induce damage to the retina via several different mechanisms. At low concentrations, A2E inhibits normal proteolysis in lysosomes (Holz et al., Invest. Ophthalmol. Vis. Sci. 40:737-43 (1999)). At higher, sufficient concentrations, A2E may act as a positively charged lysosomotropic detergent, dissolving cellular membranes, and may alter lysosomal function, release proapoptotic proteins from mitochondria, and ultimately kill the RPE cell (see, e.g., Eldred et al., supra; Sparrow et al., Invest. Ophthalmol. Vis. Sci. 40:2988-95 (1999); Holz et al., supra; Finneman et al., Proc. Natl. Acad. Sci. USA 99:3842-347 (2002); Suter et al., J. Biol. Chem. 275:39625-30 (2000)). A2E is phototoxic and initiates blue light-induced apoptosis in RPE cells (see, e.g., Sparrow et al., Invest. Ophthalmol. Vis. Sci. 43:1222-27 (2002)). Upon exposure to blue light, photooxidative products of A2E are formed (e.g., epoxides) that damage cellular macromolecules, including DNA (Sparrow et al., J. Biol. Chem. 278(20): 18207-13 (2003)). A2E self-generates singlet oxygen that reacts with A2E to generate epoxides at carbon-carbon double bonds (Sparrow et al., supra). Generation of oxygen reactive species upon photoexcitation of A2E causes oxidative damage to the cell, often resulting in cell death. An indirect method of blocking formation of A2E by inhibiting biosynthesis of the direct precursor of A2E, all-trans-retinal, has been described (see U.S. Published Patent Application 2003/0032078). However, the usefulness of this method is limited because generation of all-trans retinal is an important component of the visual cycle. Other therapies described include neutralizing damage caused by oxidative radical species by using superoxide-dismutase mimetics (see, e.g., U.S. Published Patent Application No. 2004/0116403) and inhibiting A2E-induced cytochrome C oxidase in retinal cells with negatively charged phospholipids (see, e.g., U.S. Published Patent Application No. 2003/0050283).

By contrast, the present invention provides compositions that directly interact with or target A2E. In one embodiment, the invention provides an antibody that specifically binds to A2E (and to isomers thereof) that is useful for treating retinal neuronal diseases. A2E includes A2E isomers, for example, iso-A2E (13-Z photo-isomer of A2E (see, e.g., Parish et al., Proc. Natl. Acad. Sci. USA 95:14609-13 (1998); Ben-Shabat et al., Angew. Chem. Int. Ed. 41:814-17 (2002)). The antibodies described herein may be useful for preventing, reducing, inhibiting, or decreasing accumulation (i.e., deposition) of A2E in the RPE. Without wishing to be bound by theory, because the RPE is critical for the maintenance of the integrity of photoreceptor cells, preventing, reducing, or inhibiting damage to the RPE may enhance the survival (or increase cell viability or inhibit degeneration) of retinal neuronal cells, particularly, photoreceptor cells. An antibody that binds specifically to A2E may also reduce, inhibit, prevent, or decrease one or more toxic effects of A2E that result in retinal neuronal cell (including a photoreceptor cell) damage, loss, or neurodegeneration, or in some manner decrease retinal neuronal cell viability. Such toxic effects include induction of apoptosis, self-generation of singlet oxygen and generation of oxygen reactive species; self-generation of singlet oxygen to form A2E-epoxides that induce DNA lesions, thus damaging cellular DNA and inducing cellular damage; dissolving cellular membranes; altering lysosomal function; and effecting release of proapoptotic proteins from mitochondria.

The present invention also provides A2E, or a derivative thereof, that is conjugated to a carrier, such as a hapten peptide or polypeptide carrier, for use as an immunogen or as a vaccine. Such an A2E immunoconjugate may be useful for inducing an immune response, including a cellular or a humoral response or a combination of both that is specific for A2E (and isomers of A2E) and which also may include an innate response. An immune response, which is described herein, may thus result in the prevention, reduction, inhibition, or decrease of the accumulation or deposition of A2E in a retinal neuronal cell or a RPE cell (and therefore inhibit or decrease degeneration of a retinal cell, enhance survival of a retinal cell including a retinal neuronal cell, or increase cell viability of a retinal cell including a retinal neuronal cell), and/or decrease the amount of A2E present or formed in a retinal neuronal cell, and/or reduce one or more toxic effects of A2E described herein that reduce viability of a retinal cell, such as a retinal neuronal cell (e.g., a photoreceptor cell). Accordingly, such compositions may be useful for treatment (including prophylaxis) of a retinal neuronal disease, such as macular degeneration.

An A2E immunoconjugate administered to a subject acts as an immunogen or a vaccine to induce an immune response, which includes an immune response that is specific for A2E and may also include an innate immune response. A specific immune response is due to lymphocytes, both B cell and T cell lymphocytes. In general, an immune response has been characterized as either a humoral response, in which antibodies specific for antigens are produced by differentiated B lymphocytes known as plasma cells, or a cell mediated response, in which various types of T lymphocytes act to eliminate antigens by a number of mechanisms. For example, helper T cells that are capable of recognizing specific antigens may respond by releasing soluble mediators such as cytokines to recruit additional cells of the immune system to participate in an immune response. Also, cytotoxic T cells that are also capable of specific antigen recognition may respond by binding to and destroying or damaging an antigen-bearing cell or particle.

An immune response in a host or subject that is specific for A2E may be determined by any number of well-known immunological methods described herein and with which those having ordinary skill in the art will be readily familiar. Such assays include, but need not be limited to, to in vivo or in vitro determination of soluble antibodies; soluble mediators such as cytokines (e.g., IFN-γ, IL-2, IL-4, and TGF-β), lymphokines, chemokines, hormones, growth factors, and the like, as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; cellular differentiation by cells of the immune system, including altered surface antigen expression profiles or the onset of apoptosis (programmed cell death). Procedures for performing these and similar assays are may be found, for example, in Lefkovits (Immunology Methods Manual: The Comprehensive Sourcebook of Techniques, 1998). See also Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein).

For example, antibodies that are specific for A2E that are produced in response to the A2E conjugate can be detected by one or more immunoassays, for example, an ELISA, immunoprecipitation, immunoblotting, radioimmunoassay, and the like, with which a skilled artisan is familiar. The class or classes of immunoglobulin (e.g., IgG, IgM, IgA, IgD, IgE) produced in response to the A2E conjugate may also be determined by such methods. Production of specific antibodies that bind to A2E may be detected in serum; production of intraocular antibodies may also be detected, for example, in tears, aqueous humor, and vitreous humor.

The immune response that is induced by an A2E conjugate as described herein may include a mucosal immune response. Lacrimal glands are the primary exocrine tissue for the ocular surface and are considered to be part the mucosal immune system (see, e.g., Knop et al., Invest. Ophthalmol. Vis. Sci. 42:566-574 (2001)). Antibodies produced within the ocular compartment include IgA antibodies, and secretory IgA is a major component of lacrimal fluid, the presence of which can be determined according to immunoassays described herein and practiced in the art. (see, e.g., Meek et al., Prog. Retin. Eye Res. 22:391-415 (2003); Saitoh-Inagawa et al., Invest. Ophthalmol. Vis. Sci. 41:138-44 (2000)). Delivery of an A2E immunogen intranasally or nasopharyngeally may enhance the mucosal immune response.

Retinal Cells

The exemplary long-term in vitro cell culture system described herein permits and promotes the survival in the culture of mature retinal cells, including retinal neurons, for at least 2-4 weeks, over 2 months, or for as long as 6 months. Retinal cells are isolated from non-embryonic, non-tumorigenic tissue and have not been immortalized by any method such as, for example, transformation or infection with an oncogenic virus. The cell culture system may comprise all the major retinal neuronal cell types (photoreceptors, bipolar cells, horizontal cells, amacrine cells, and ganglion cells), and also may include other mature retinal cells such as retinal pigmented epithelial cells and Müller glial cells.

The retina of the eye is a thin, delicate layer of nervous tissue. The major landmarks of the retina are the area centralis in the posterior portion of the eye and the peripheral retina in the anterior portion of the eye. The retina is thickest near the posterior sections and becomes thinner near the periphery. The area centralis is located in the posterior retina and contains the fovea and foveola and, in primates, contains the macula. The foveola contains the area of maximal cone density and, thus, imparts the highest visual acuity in the retina. The foveola is contained within the fovea, which is contained within the macula.

The peripheral or anterior portion of the retina increases the field of vision. The peripheral retina extends anterior to the equator of the eye and is divided into four regions: the near periphery (most posterior), the mid-periphery, the far periphery, and the ora serrata (most anterior). The ora serrata denotes the termination of the retina.

The term neuron (or nerve cell) as understood in the art and used herein denotes a cell that arises from neuroepithelial cell precursors. Mature neurons (i.e., fully differentiated cells from an adult) display several specific antigenic markers. Neurons may be classified functionally into three groups: (1) afferent neurons (or sensory neurons) that transmit information into the brain for conscious perception and motor coordination; (2) motor neurons that transmit commands to muscles and glands; and (3) interneurons that are responsible for local circuitry; and (4) projection interneurons that relay information from one region of the brain to anther region and therefore have long axons. Interneurons process information within specific subregions of the brain and have relatively shorter axons. A neuron typically has four defined regions: the cell body (or soma); an axon; dendrites; and presynaptic terminals. The dendrites serve as the primary input of information from other cells. The axon carries the electrical signals that are initiated in the cell body to other neurons or to effector organs. At the presynaptic terminals, the neuron transmits information to another cell (the postsynaptic cell), which may be another neuron, a muscle cell, or a secretory cell.

The retina is composed of several types of neuronal cells. As described herein, the types of retinal neuronal cells that may be cultured in vitro by this method include photoreceptor cells, ganglion cells, and interneurons such as bipolar cells, horizontal cells, and amacrine cells. Photoreceptors are specialized light-reactive neural cells and comprise two major classes, rods and cones. Rods are involved in scotopic or dim light vision, whereas photopic or bright light vision originates in the cones by the presence of trichromatic pigments. Many neurodegenerative diseases that result in blindness, such as macular degeneration, retinal detachment, retinitis pigmentosa, diabetic retinopathy, etc, affect photoreceptors.

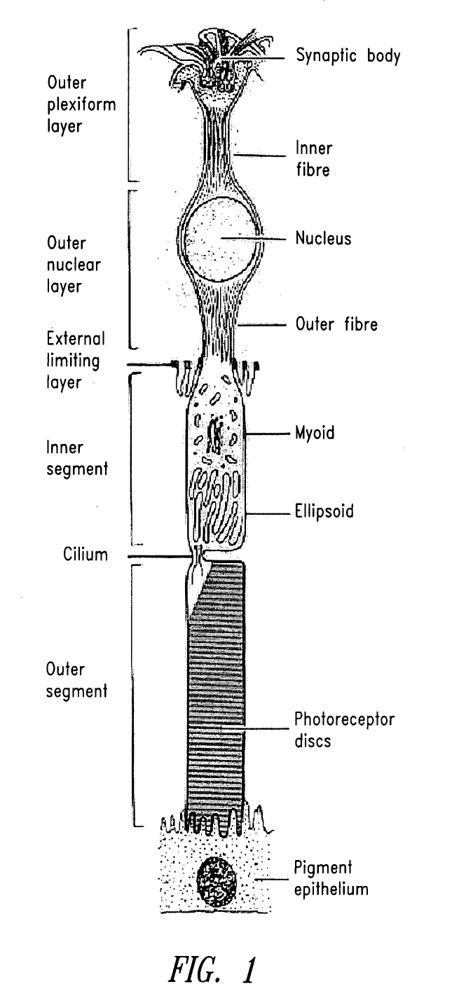

Extending from their cell bodies, the photoreceptors have two morphologically distinct regions, the inner and outer segments (see FIG. 1). The outer segment lies furthermost from the photoreceptor cell body and contains disks that convert incoming light energy into electrical impulses (phototransduction). As shown in FIG. 1, the outer segment is attached to the inner segment with a very small and fragile cilium. The size and shape of the outer segments vary between rods and cones and are dependent upon position within the retina. See Eye and Orbit, 8^{th} Ed., Bron et al., (Chapman and Hall, 1997).

Ganglion cells are output neurons that convey information from the retinal interneurons (including horizontal cells, bipolar cells, amacrine cells) to the brain. Bipolar cells are named according to their morphology, and receive input from the photoreceptors, connect with amacrine cells, and send output radially to the ganglion cells. Amacrine cells have processes parallel to the plane of the retina and have typically inhibitory output to ganglion cells. Amacrine cells are often subclassified by neurotransmitter or neuromodulator or peptide (such as calretinin or calbindin) and interact with each other, with bipolar cells, and with photoreceptors. Bipolar cells are retinal interneurons that are named according to their morphology; bipolar cells receive input from the photoreceptors and sent the input to the ganglion cells. Horizontal cells modulate and transform visual information from large numbers of photoreceptors and have horizontal integration (whereas bipolar cells relay information radially through the retina).

Other retinal cells that may be present in the retinal cell cultures described herein include glial cells, such as Müller glial cells, and retinal pigmented epithelial cells (RPE). Glial cells surround nerve cell bodies and axons. The glial cells do not carry electrical impulses but contribute to maintenance of normal brain function. Müller glia, the predominant type of glial cell within the retina, provide structural support of the retina and are involved in the metabolism of the retina (e.g., contribute to regulation of ionic concentrations, degradation of neurotransmitters, and remove certain metabolites (see, e.g., Kljavin et al., J. Neurosci. 11:2985 (1991))). Müller's fibers (also known as sustentacular fibers of retina) are sustentacular neuroglial cells of the retina that run through the thickness of the retina from the internal limiting membrane to the bases of the rods and cones where they form a row of junctional complexes.

Retinal pigmented epithelial (RPE) cells form the outermost layer of the retina, nearest the blood vessel-enriched choroids. RPE cells are a type of phagocytic epithelial cell, functioning like macrophages, that lies below the photoreceptors of the eye. The dorsal surface of the RPE cell is closely apposed to the ends of the rods, and as discs are shed from the rod outer segment they are internalized and digested by RPE cells. RPE cells also produce, store, and transport a variety of factors that contribute to the normal function and survival of photoreceptors. Another function of RPE cells is to recycle vitamin A as it moves between photoreceptors and the RPE during light and dark adaptation.

A2E-Like Compounds, A2E Derivatives, and A2E Conjugates

Retinoid N-retinylidene-N-retinyl-ethanolamine (A2E) is a small organic molecule. A2E may include any one or more of A2E isomers including, for example, iso-A2E (13-Z photo-isomer of A2E (see, e.g., Parish et al., Proc. Natl. Acad. Sci. USA 95:14609-13 (1998); Ben-Shabat et al., Angew. Chem. Int. Ed. 41:814-17 (2002)), or may include all isoforms of A2E. Retinoids are a class of compounds that have four isoprenoid units joined in a head-to-tail manner. All retinoids may be formally derived from a monocyclic parent compound containing five carbon-carbon double bonds and a functional group at the terminus of the acyclic portion. Examples of retinoid molecules include retinal, vitamin A, and retinoic acid (see also, e.g., International Union of Pure and Applied Chemistry (IUPAC) definitions). As described herein, A2E is a product of condensation of retinal with ethanolamine derivatives.

A2E conjugates as described herein may be used as A2E immunogens for generation of antibodies that specifically bind to A2E. An A2E conjugate may also be used as a vaccine in a composition for inducing an immune response in a host. The immune response that is induced may comprise a humoral response or humoral immunity, that is, proliferation and activation of B lymphocytes (B cells) that produce antibodies that bind specifically to A2E. The A2E immunogen may also induce a cellular response or cellular immunity that is mediated for the most part by T lymphocytes (T cells). The effect of inducing an immune response specific for A2E includes decreasing, inhibiting, reducing, or preventing accumulation of A2E in or near retinal neuronal cells and RPE cells and thereby reducing, abrogating, preventing, or decreasing deleterious or toxic effects that result from accumulation of A2E in the eye. The effect of decreasing accumulation of A2E (decreasing in a statistically or biologically significant manner) in the eye (in or near retinal neuronal cells and RPE cells) inhibits degeneration of a retinal cell, enhances retinal cell survival and/or increases retinal cell viability, which prevents, inhibits, slows the progression of, or reduces the symptoms associated with, an ophthalmic disease, for example, macular degeneration. A retinal cell includes a retinal neuronal cell and mature retinal cells such as retinal pigmented epithelial cells and Müller glial cells as described herein. Thus, a method is provided herein for treating an ophthalmic disease in a subject, which method comprises administering to the subject a composition that comprises an A2E conjugate as described herein. An ophthalmic disease includes macular degeneration, glaucoma, diabetic retinopathy, retinal detachment, retinal blood vessel occlusion, retinitis pigmentosa, optic neuropathy, inflammatory retinal disease, a retinal disorder associated with Alzheimer's disease, and a retinal disorder associated with multiple sclerosis. In a particular embodiment the ophthalmic disease is macular degeneration.

A2E is a small, organic molecule (approximately 590 daltons) that has at least one antigenic determinant (or epitope), that is, the molecule can bind to and be recognized by an antibody; however, immunization with A2E is unlikely to provoke an antibody response in a host or to provoke only a weak immune response. Such a molecule is referred to in the art as a hapten. Immunogenicity of A2E can be achieved by attaching or conjugating A2E, the hapten, to a larger molecule called the carrier. Carrier molecules are often carrier polypeptides but also can include a sugar or lipid. Exemplary carrier polypeptides include but are not limited to keyhole limpet hemocyanin (KLH), bovine serum albumin (BSA), ovalbumin, tetanus toxoid (e.g., tetanus toxin Fragment C (see, e.g., Bowers et al., Neurobiol. Aging 26:393-407 (2005)), diphtheria toxoid, E. coli heat-labile enterotoxin B subunit, polyglutamate, glucose oxidase, rabbit serum albumin (RSA), sperm whale myoglobin, human thyroglobulin, Pasteurella haemolytica leukotoxin (LKT) polypeptide (see, e.g., U.S. Pat. No. 5,969,126).

The attachment or conjugation of a hapten to a carrier, such as a carrier polypeptide, is most often covalent but may be ionic, the result of van der Waals forces, or via hydrogen bridges. Conjugation of an A2E derivative or A2E-like molecule to a carrier (particularly to an amino acid of a carrier) may therefore be achieved under conditions and for a time sufficient that permit a covalent bonding reaction or by admixing an A2E derivative or A2E-like molecule and the carrier under conditions appropriate and for a time sufficient to permit a non-covalent interaction or linking to occur (see, e.g., U.S. Pat. No. 6,699,474; U.S. Pat. No. 6,383,490; U.S. Pat. No. 6,773,891; Amara et al., Neurosci. Lett. 185:147-50 (1995)).

The hapten A2E (also called herein a A2E derivative, or A2E-like compound) may be attached directly to an amino acid of the carrier or attached to the carrier via a linker. The carrier may be a peptide, such as a hapten peptide, or may be a polypeptide carrier. In a specific embodiment the hapten A2E may be attached or conjugated to a hapten peptide that is then attached or conjugated to a polypeptide carrier. In certain embodiments, the A2E hapten may be conjugated to the carrier via a linker. The linker (which may also be referred to in the art as a spacer) is non-immunogenic and is typically an alkyl chain or polyethylene glycol or oligoethylene glycol chain. Preferably the linker is hydrophilic in nature and not hydrophobic, which might alter the geometry of the A2E molecule. The linker is attached to A2E at a position of A2E that preserves the structural features and geometry of A2E, such that an antibody generated by immunizing a host or animal with the A2E carrier conjugate will specifically bind molecules of A2E under physiological conditions, that is, in a cell, biological sample, or in a host or subject (see, e.g., Zhou et al., J. Immunol. Meth. 138:211-23 (1991)).

As described herein, an A2E like compound or a derivative of A2E that has a suitable reactive group may be attached directly to an amino acid of a carrier (such as a carrier polypeptide) or attached to a carrier via a linker (spacer) for use as an immunogenic conjugate. The reactive group may be nucleophilic or electrophilic. These derivatives may be conjugated to a carrier directly in the presence of activating reagents or in the presence of bifunctional crosslinking reagents according to methods described herein and known in the art (see, e.g., Pierce Biotechnology, Inc. (Rockville, Ill.), which offers a selection of activating reagents and crosslinking reagents). At least one molecule of an A2E hapten is conjugated to a carrier (such as a carrier polypeptide) but additional molecules of A2E hapten may be conjugated to one molecule of a carrier. The number of A2E hapten molecules that are conjugated to a molecule of the carrier may depend on the particular carrier and the conjugation chemistry used to perform the conjugation (attachment). By way of example, a thiol derivative of A2E can be prepared that permits attachment to a maleimide-activated immunogenic protein, such as Imject® keyhole limpet hemocyanin (KLH) (Pierce Biotechnology, Inc.). Another exemplary method for conjugating A2E to a carrier polypeptide comprises preparing an A2E derivative that has an amino group and coupling the derivative to the an amino acid of the carrier via reductive amination using glutaraldehyde. Crosslinking reagents such as glutaraldehyde provide an additional linker or spacer moiety, in addition to the linker as described herein that is being attached to A2E. Alternatively, an A2E derivative that has a carboxy group can be conjugated to an amino acid of the carrier by using a suitable activating reagent. Such activating reagents include but are not limited to DCC (N,N′-dicylohexylcarbodiimide), EDC (N-(3-Dimethylaminopropyl) N′-ethylcarbodiimide), or pentafluorophenyl trifluoroacetate.

The position (i.e., point of attachment) on a small molecule to which a carrier molecule such as a carrier polypeptide or a linker is attached is determined according to which position when attached to another moiety does not result in perturbation of the structural features of a small molecule, that is, the structural integrity of the small molecule is preserved after attachment to a carrier or to a carrier via a linker. Maintaining the structural features or integrity of A2E may be important for eliciting and/or generating an antibody that is specific for A2E, minimizing cross-reactivity with other retinoid molecules such as vitamin A and retinal (see, e.g., Zhou et al., J. Immunol. Meth. 138:211-23 (1991)).

A positively charged six-member pyridyl ring is a unique feature of A2E that distinguishes it from other retinoid molecules. In one embodiment, the invention provides A2E-like compounds and A2E derivatives used for making an A2E immunoconjugate (i.e., A2E attached to a carrier such as a carrier polypeptide) that retains this pyridyl ring feature. In certain embodiments, the linker is attached to the nitrogen atom of the pyridyl ring of the A2E molecule, which preserves the original geometry of the molecule as well as the positive charge. The invention provides the following A2E-like compounds and A2E derivatives that are useful for preparing an A2E immunoconjugate.

In one embodiment, an A2E-like compound has the following structure (1):

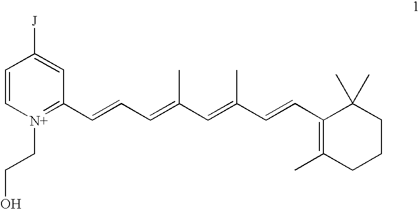

or a pharmaceutically acceptable acid addition salt thereof;

wherein

J is -Z¹-Y, -Z²-R⁰—Y, -Z²-R⁰-Z³-Y, or -Z²-Y′—R⁰,

Z¹ is a divalent C₁-C₄₀ alkyl,

Z² is a divalent C₁-C₄₀ alkyl,

Z³ is a polyethylene glycol having the formula —(CH₂CH₂O)ₙCH₂CH₂— wherein n=2-12, or —R⁴—CH₂—, wherein R⁴ is C₁-C₄₀ alkylene or C₁-C₄₀ heteroalkylene;

R⁰ is a monovalent or divalent optionally substituted homocycle, aryl, heteroaryl, or heterocycle; and

wherein Y is a monovalent electrophilic or nucleophilic moiety suitable for covalent attachment of the compound to an amino acid, and wherein Y′ is a divalent electrophilic or nucleophilic moiety suitable for covalent attachment of the compound to an amino acid.

In a specific embodiment, J is -Z¹-Y, and in another specific embodiment J is -Z²-R⁰—Y. In another specific embodiment, J is -Z²-R⁰-Z³-Y.

In certain embodiments, Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl. In another specific embodiment, Y is —OH, —SH, —NH₂, —C(═O)OH, or oxo; in a particular specific embodiment, Y is —NH₂.

In another certain embodiment, J is -Z²-Y′—R⁰, wherein Y′ is —O—. In still another certain embodiment, Y′ is is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

In a specific embodiment, the A2E-like compound is a compound having structure 1, wherein J is -Z²-R⁰-Z³-Y and Z³ is a polyethylene glycol having the formula —(CH₂CH₂O)ₙCH₂CH₂— wherein n=2-12. In certain embodiments, Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl. In another specific embodiment, Y is —OH, —SH, —NH₂, —C(═O)OH, or oxo, and in a particular specific embodiment, Y is —NH₂.

In yet another specific embodiment, the A2E-like compound is a compound having structure 1, wherein J is -Z²-R⁰-Z³-Y and Z³ is —R⁴—CH₂—. In a specific embodiment R⁴ is C₁-C₄₀ alkylene, and in another specific embodiment, R⁴ is C₁-C₄₀ heteroalkylene. In still another specific embodiment, R⁴ is C₁-C₂₀ alkylene, and yet another specific embodiment, R⁴ is C₁-C₂₀ heteroalkylene. In other specific embodiments, when R⁴ is C₁-C₄₀ heteroalkylene or C₁-C₂₀ heteroalkylene, the heteroalkylene comprises at least one of amide, disulfide, —O—, —S—, and sulfonamide (i.e., a substitution pattern selected). In certain embodiments, Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl. In another specific embodiment, Y is —OH, —SH, —NH₂, —C(═O)OH, or oxo, and in a particular specific embodiment, Y is —NH₂.

In another embodiment, the A2E-like compound is a compound having structure 1, or a pharmaceutically acceptable acid addition salt thereof, wherein J is -Z¹-Y or -Z²-R⁰—Y, wherein each of Z¹ and Z² is a divalent C₁-C₄₀ alkyl, and R⁰ is a monovalent or divalent optionally substituted homocycle, aryl, heteroaryl, or heterocycle; wherein Y is a monovalent electrophilic or nucleophilic moiety suitable for covalent attachment of the compound to an amino acid. In one specific embodiment, J is -Z¹-Y, and in another specific embodiment J is -Z²-R⁰—Y. In certain embodiments, Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl. In another specific embodiment, Y is —OH, —SH, —NH₂, —C(═O)OH, or oxo, and in a particular specific embodiment, Y is —NH₂.

In certain embodiments, R⁰ may be optionally substituted as defined herein. In other certain embodiments, R⁰ may be optionally substituted by one or more of the following substituents: —O—R″, —O—R′, —O—R″—CH₂—NR′, —O—CH₂—R″—NR′, —O—R″—CH₂, —O—CH₂—R″, —S—R″, —S—R′, —S—R″—CH₂—NR′, —S—CH₂—R″—NR′, S—R″—CH₂, —S—CH₂—R″, —C(═O)NR′—R″, —C(═O)NR′—R″—NR′, and the like, and combinations thereof, including where each R′ is independently selected from hydrogen, alkyl, alkanyl, alkenyl, alkynyl, aryl, arylalkyl, heteroaryl and heteroarylalkyl, as defined herein, and wherein R″ is a straight or branched alkylene chain of 1 to 40 carbon atoms. In other certain specific embodiments, the substituent is divalent and joins the homocycle, aryl, heteroaryl, or heterocycle with Z³ or with the Y group. In other certain specific embodiments, the substituent is monovalent.

In other embodiments of an A2E-like compound, wherein the compound has structure 1, J is -Z²-R⁰-Z³-Y, and -Z²-R⁰-Z³- has the following structure (1):

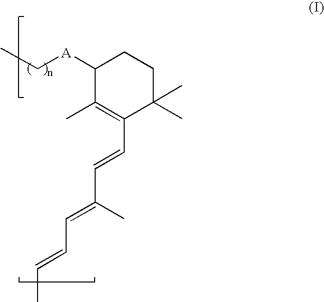

wherein n=0-12 when A is a direct bond; wherein n=1-10 when A is —O—, —NH—, —S—, —S—S—, —C(═O)NH, —NHC(═O)—, —NHC(═O)NH—, —OC(═O)—, or —C(═O)O—;

and wherein Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

In another embodiment, J is -Z²-R⁰-Z³-Y, and -Z²-R⁰-Z³- has the following structure (II):

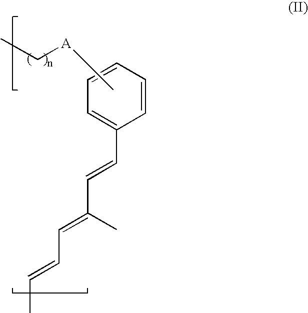

wherein n=0-12 when A is a direct bond; wherein n=1-10 when A is —O—, —NH—, —S—, —S—S—, —C(═O)NH, —NHC(═O)—, —NHC(═O)NH—, —OC(═O)—, or —C(═O)O—; and wherein Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

In another embodiment of a compound having structure 1, j is -Z²-Y′—R⁰—, and -Z²-Y′—R⁰— has the following structure (III):

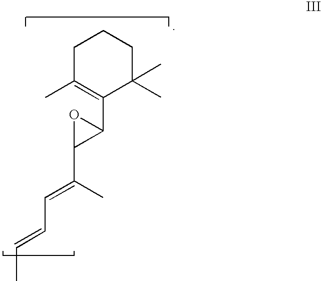

In still another embodiment of a compound having structure 1, J is -Z²-R⁰—Y, and -Z²-R⁰- has the following structure (IV):

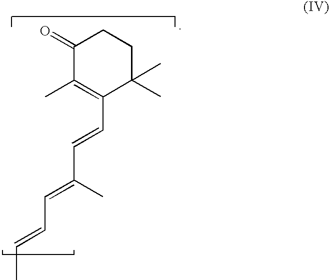

In a particular embodiment, an A2E-like compound having structure 1 has the structure 1(a) (also described herein as compound 9)

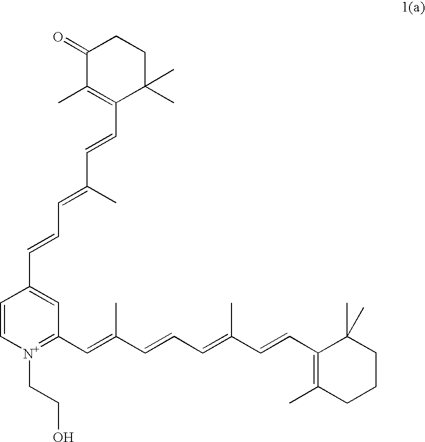

In another particular embodiment, an A2E-like compound having structure 1 has the specific structure 1(b) (also described herein as compound 25):

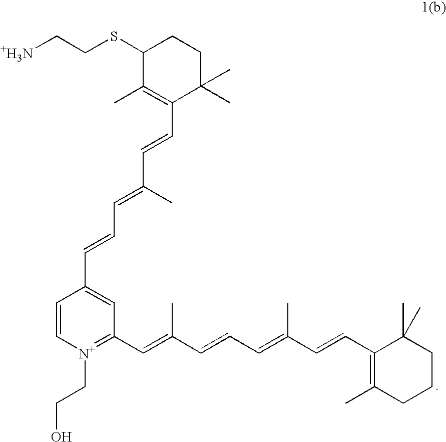

In another particular embodiment, an A2E-like compound has the structure 1 (c) (also described herein as compound 34):

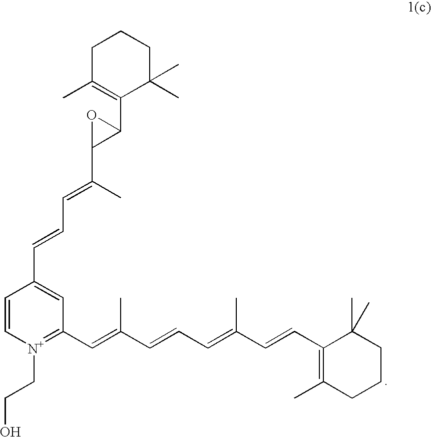

In another embodiment, an A2E-like compound has the following structure (2):

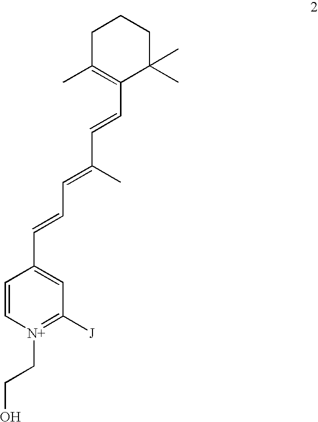

or a pharmaceutically acceptable acid addition salt thereof,

wherein

J is -Z¹-Y, -Z²-R⁰—Y, -Z²-R⁰-Z³-Y, or -Z²-Y′—R⁰,

Z¹ is a divalent C₁-C₄₀ alkyl,

Z² is a divalent C₁-C₄₀ alkyl,

Z³ is a polyethylene glycol having the formula —(CH₂CH₂O)ₙCH₂CH₂— wherein n=2-12, or —R⁴—CH₂—, wherein R⁴ is C₁-C₄₀ alkylene or C₁-C₄₀ heteroalkylene;

R⁰ is an monovalent or divalent optionally substituted homocycle, aryl, heteraryl, or heterocycle; and

wherein Y is a monovalent electrophilic or nucleophilic moiety suitable for covalent attachment of the compound to an amino acid, and wherein Y′ is a divalent electrophilic or nucleophilic moiety suitable for covalent attachment of the compound to an amino acid.

In a specific embodiment, J is -Z¹-Y, and in another specific embodiment J is -Z²-R⁰—Y. In another specific embodiment, J is -Z²-R⁰-Z³-Y.

In certain embodiments, Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl. In another specific embodiment, Y is —OH, —SH, —NH₂, —C(═O)OH, or oxo; in a particular specific embodiment, Y is —NH₂.

In another certain embodiment, J is -Z²-Y′—R⁰, wherein Y′ is —O—. In still another certain embodiment, Y′ is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

In a specific embodiment, the A2E-like compound is a compound having structure 1, wherein J is -Z²-R⁰-Z³-Y and Z³ is a polyethylene glycol having the formula —(CH₂CH₂O)ₙCH₂CH₂— wherein n=2-12. In certain embodiments, Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl. In another specific embodiment, Y is —OH, —SH, —NH₂, —C(═O)OH, or oxo, and in a particular specific embodiment, Y is —NH₂.

In yet another specific embodiment, the A2E-like compound is a compound having structure 1, wherein J is -Z²-R⁰-Z³-Y and Z³ is —R⁴—CH₂—. In a specific embodiment R⁴ is C₁-C₄₀ alkylene, and in another specific embodiment, R⁴ is C₁-C₄₀ heteroalkylene. In still another specific embodiment, R⁴ is C₁-C₂₀ alkylene, and yet another specific embodiment, R⁴ is C₁-C₂₀ heteroalkylene. In other specific embodiments, when R⁴ is C₁-C₄₀ heteroalkylene or C₁-C₂₀ heteroalkylene, the heteroalkylene comprises at least one of amide, disulfide, —O—, —S—, and sulfonamide (i.e., a substitution pattern). In certain embodiments, Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl. In another specific embodiment, Y is —OH, —SH, —NH₂, —C(═O)OH, or oxo, and in a particular specific embodiment, Y is —NH₂.

In another embodiment, the A2E-like compound is a compound having structure 1, or a pharmaceutically acceptable acid addition salt thereof, wherein J is -Z¹-Y or -Z²-R⁰—Y, wherein each of Z¹ and Z² is a divalent C₁-C₄₀ alkyl, and R⁰ is a monovalent or divalent optionally substituted homocycle, aryl, heteroaryl, or heterocycle; wherein Y is a monovalent electrophilic or nucleophilic moiety suitable for covalent attachment of the compound to an amino acid. In one specific embodiment, J is -Z¹-Y, and in another specific embodiment J is -Z²-R⁰—Y. In certain embodiments, Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl. In another specific embodiment, Y is —OH, —SH, —NH₂, —C(═O)OH, or oxo, and in a particular specific embodiment, Y is —NH₂.

In certain embodiments, R⁰ may be optionally substituted as defined herein. In other certain embodiments, R⁰ may be optionally substituted by one or more of the following substituents: —O—R″, —O—R′, —O—R″—CH₂—NR′, —O—CH₂—R″—NR′, —O—R″—CH₂, —O—CH₂—R″, —S—R″, —S—R′, —S—R″—CH₂—NR′, —S—CH₂—R″—NR′, S—R″—CH₂, —S—CH₂—R″, —C(═O)NR′—R″, —C(═O)NR′—R″—NR′, and the like, and combinations thereof, including where each R′ is independently selected from hydrogen, alkyl, alkanyl, alkenyl, alkynyl, aryl, arylalkyl, heteroaryl and heteroarylalkyl, as defined herein, and wherein R″ is a straight or branched alkylene chain of 1 to 40 carbon atoms. In other certain specific embodiments, the substituent is divalent and joins the homocycle, aryl, heteroaryl, or heterocycle with Z³ or with the Y group. In other certain specific embodiments, the substituent is monovalent.

In another embodiment, wherein the A2E like compound has a structure of structure 2, J is -Z²-R⁰-Z³-Y, and -Z²-R⁰-Z³- has the structure (V):

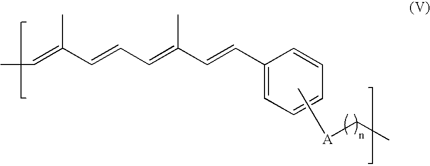

wherein n=0-12 when A is a direct bond; wherein n=1-10 when A is —O—, —NH—, —S—, —S—S—, —C(═O)NH, —NHC(═O)—, —NHC(═O)NH—, —OC(═O)—, or —C(═O)O—; and wherein Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

In another embodiment, wherein the A2E like compound has a structure of structure 2, J is -Z²-R⁰-Z³-Y, and -Z²-R⁰-Z³- has the following structure (VI):

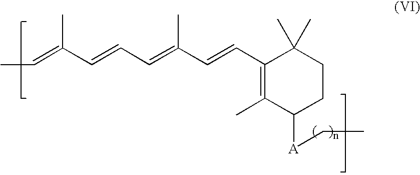

wherein n=0-12 when A is a direct bond; wherein n=1-10 when A is —O—, —NH—, —S—, —S—S—, —C(═O)NH, —NHC(═O)—, —NHC(═O)NH—, —OC(═O)—, or —C(═O)O—; and wherein Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

In another embodiment, wherein the A2E like compound has a structure of structure 2, J is -Z²-Y′—R⁰, and -Z²-Y′—R⁰ has the following structure (VII):

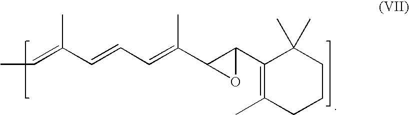

In another embodiment, wherein the A2E like compound has a structure of structure 2, J is -Z²-R⁰—Y, and -Z²-R⁰—Y has the following structure (VIII):

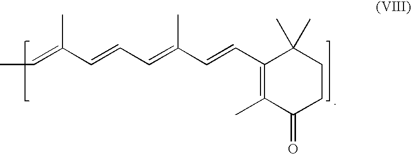

In a specific embodiment, the A2E like compound of structure 2 has the structure 2(a) (also referred to herein as compound 17) 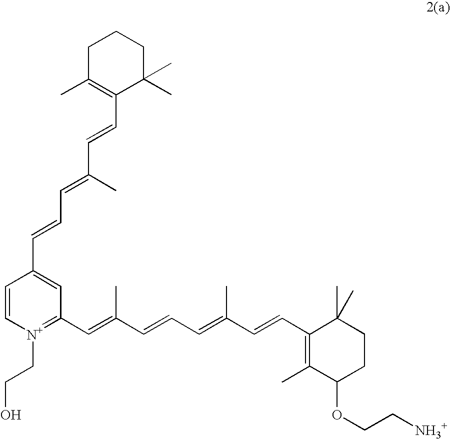
or structure 2(b) (also referred to herein as compound 33)

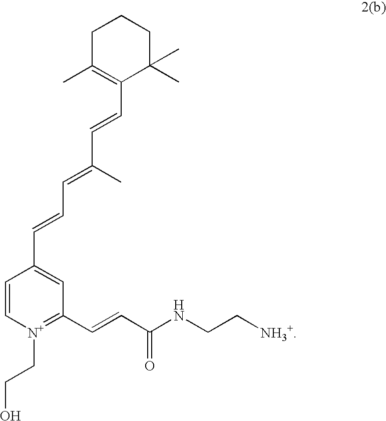

In another embodiment, an A2E like compound has the following structure (3):

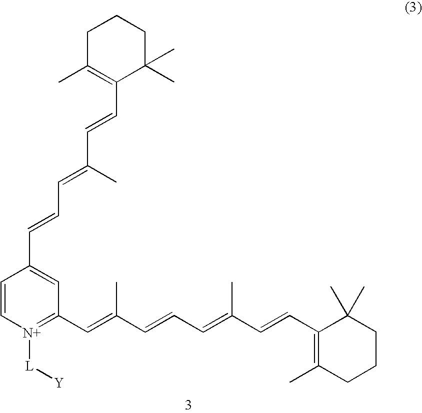

or a pharmaceutically acceptable acid addition salt thereof,

wherein L is a divalent linker group -R₁-, -R₂-, or -R₃-, and

R₁ is divalent C₁-C₆ alkyl;

R₂ is C₁-C₄₀ alkylene or C₁-C₄₀ heteroalkylene; and

R₃ is a polyethylene glycol having the formula —(CH₂CH₂O)ₙCH₂CH₂— wherein n=2-12;

and wherein Y is an electrophilic or nucleophilic moiety suitable for reaction of the compound with an amino acid.

In one embodiment, L is -R₁-; in another embodiment, L is -R₂-; and in still another embodiment, L is -R₃-. In another certain embodiment, R₃ is a polyethylene glycol moiety having the formula —(CH₂CH₂O)ₙCH₂CH₂—, wherein n=1 to 4.

In another certain embodiment, L is -R₂-, wherein R₂ is C₁-C₄₀ alkylene, and in another embodiment, R₂ is C₁-C₄₀ heteroalkylene. In another particular embodiment, R₂ is C₁-C₂₀ alkylene, and in another embodiment, R₂ is C₁-C₂₀ heteroalkylene. In certain specific embodiments when R₂ is C₁-C₄₀ heteroalkylene or R₂ is C₁-C₂₀ heteroalkylene, the heteroalkylene comprises at least one of amide, disulfide, —O—, —S—, and sulfonamide (i.e., substitution pattern).

In certain embodiments, Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

In a specific embodiment, R₃ is —(CH₂CH₂O)₂CH₂CH₂— and Y is —NH₂. In another specific embodiment, R₃ is —(CH₂CH₂O)₂CH₂CH₂— and Y is —SH. In still another specific embodiment, R₃ is —(CH₂CH₂O)₄CH₂CH₂— and Y is —C(═O)OH.

In specific embodiments, an A2E like compound has the structure 3(a) (also referred to herein as compound 41): 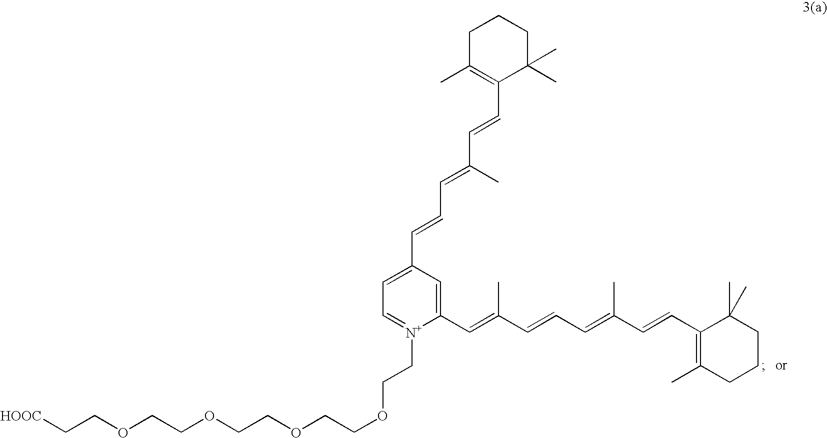
the structure 3(b) (also referred to herein as compound 44) 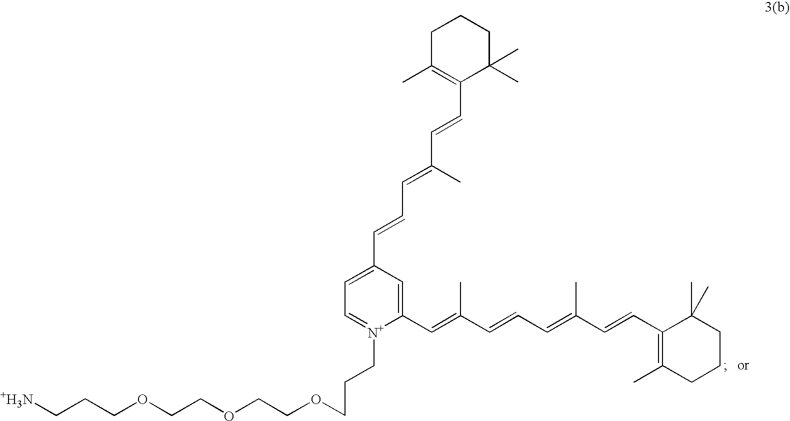
the structure 3(c) (also referred to herein as compound 39)

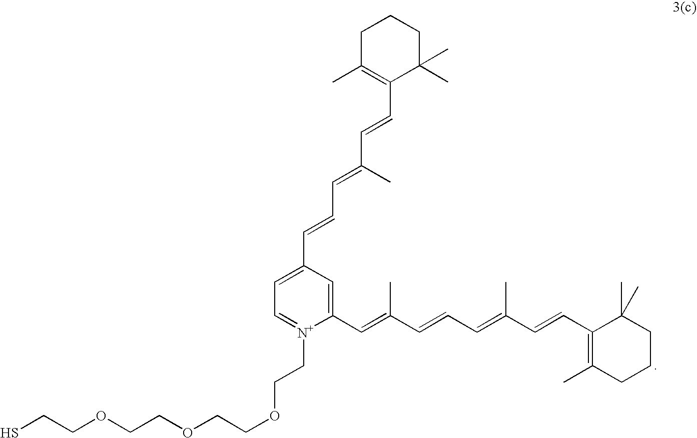

As used in the specification and appended claims, unless specified to the contrary, the following terms have the meaning indicated:

Certain chemical groups named herein are preceded by a shorthand notation indicating the total number of carbon atoms that are to be found in the indicated chemical group. For example; C₇-C₁₂ alkyl describes an alkyl group, as defined below, having a total of 7 to 12 carbon atoms, and C₄-C₁₂ heteroalkylene describes a heteroalkylene group, as defined below, having a total of 4 to 12 carbon atoms. The total number of carbons in the shorthand notation does not include carbons that may exist in substituents of the group described. It is understood that any shorthand notation of a certain range of carbons in a group includes all possible ranges, such as C₁-C₄₀ heteroalkylene is intended to include C₁-C₂₀ heteroalkylene, as well as C₂-C₂₀ heteroalkylene.

The term “substituted” in the context of alky, aryl, arylalkyl, heterocycle and heterocyclealkyl means that at least one hydrogen atom of the alky, aryl, arylalkyl, heterocycle or heterocyclealkyl moiety is replaced with a substituent. In the case of an oxo substituent (“═O”) two hydrogen atoms are replaced. A “substituent” as used within the context of this invention includes oxo, halogen, hydroxy, cyano, nitro, amino, alkylamino, dialkylamino, alkyl, alkoxy, thioalkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐR_{b}, —NRₐC(═O)OR_{b}—NRₐSO₂R_{b}, —ORₐ, —C(═O)Rₐ, —C(═O)ORₐ, —C(═O)NRₐR_{b}, —OC(═O)NRₐR_{b}, —SH, —SRₐ, —SORₐ, —S(═O)₂Rₐ, —OS(═O)₂Rₐ and —S(═O)₂ORₐ, wherein Rₐ and R_{b} are the same or different and independently hydrogen, alkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

Representative substituents include (but are not limited to) alkoxy (i.e., alkyl-O—, e.g., methoxy, ethoxy, propoxy, butoxy, pentoxy), aryloxy (e.g., phenoxy, chlorophenoxy, tolyloxy, methoxyphenoxy, benzyloxy, alkyloxycarbonylphenoxy, alkyloxycarbonyloxy, acyloxyphenoxy), acyloxy (e.g., propionyloxy, benzoyloxy, acetoxy), carbamoyloxy, carboxy, mercapto, alkylthio, acylthio, arylthio (e.g., phenylthio, chlorophenylthio, alkylphenylthio, alkoxyphenylthio, benzylthio, alkyloxycarbonyl-phenylthio), amino (e.g., amino, mono- and di-C₁-C₃ alkanylamino, methylphenylamino, methylbenzylamino, C₁-C₃ alkanylamido, acylamino, carbamamido, ureido, guanidino, nitro and cyano). Moreover, any substituent may have from 1-5 further substituents attached thereto.

“Alkyl” means a straight chain or branched, noncyclic or cyclic, unsaturated or saturated aliphatic hydrocarbon containing from 1 to 40 carbon atoms. Representative saturated straight chain alkyls include methyl, ethyl, n-propyl, n-butyl, n-pentyl, n-hexyl, and the like. Saturated branched alkyls include isopropyl, sec-butyl, isobutyl, tert-butyl, isopentyl, and the like. Representative saturated cycloalkyls (cyclic alkyls) include cyclopropyl, cyclobutyl, cyclopentyl, cyclohexyl, —CH₂cyclopropyl, —CH₂cyclobutyl, —CH₂cyclopentyl, —CH₂cyclohexyl, and the like; while unsaturated cyclic alkyls include cyclopentenyl and cyclohexenyl, and the like. Cycloalkyls, also referred to as “homocyclic rings,” include di- and poly-homocyclic rings such as decalin and adamantyl. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. Representative straight chain and branched alkynyls include acetylenyl, propynyl, I-butynyl, 2-butynyl, 1-pentynyl, 2-pentynyl, 3-methyl-1 butynyl, and the like.

“Alkylene” or “alkylene chain” refers to a straight or branched divalent, saturated or unsaturated hydrocarbon chain linking the rest of the molecule to a radical group, consisting solely of carbon and hydrogen, and having from one to forty carbon (C₁-C₄₀) atoms, for example, methylene, ethylene, propylene, n-butylene, and the like. The alkylene chain can be attached to the rest of the molecule through a single bond, a double bond or a triple bond, depending upon the valence of the atom at the point of attachment. Representative alkylene chains include ethylene, propylene, propenylene, n-butenylene, and the like.

“Heteroalkylene” is a divalent straight or branched, noncyclic or cyclic, unsaturated or saturated aliphatic hydrocarbon chain containing from 1 to 40 carbon atoms (C₁-C₄₀) wherein at least one carbon atom is replaced or substituted with a heteroatom. Heteroatoms include, but are not limited to, nitrogen, oxygen, sulfur, halogen, and phosphorous. A heteratom may, but typically does not, have the same number of valence sites as carbon. Accordingly, when a carbon atom is replaced with a heteroatom in the heteroalkylene, the number of hydrogens bonded to the heteroatom may need to be increased or decreased t match the number of valence sites of the heteroatom. For instance, if carbon (valence of four) is replaced with nitrogen (valence of three), then one of the hydrogens formerly attached to the replaced carbon must be deleted. Likewise, if carbon is replaced with halogen (valence of one), then three (i.e., all) of the hydrogens formerly bonded to the replaced carbon must be deleted. Carbons and heteroatoms within the heteroalkylene chain may be optionally oxided to their respective oxidized states. Typical substitution and replacement patterns within the heteroalkylene, include, but are not limited to, the following: —O—, —S—, —O—O—, —S—S—, —O—S—, —O—S—O—, —O—NR′—, —NR′—, —NR′—S—S, —NR′—NR′—, ═N—N═, —N═N—, —N═N—NR′—, —PH—, —P(═O)₂—, —O—P(═O)₂—, —SH₂—, —S(═O)₂—, —S(═O)—, —C(═O)—, —C(═O)O—, oxime, hydroxylamine, —NR′—S(═O)₂—, —C(═O)NR′—, —NHC(═O)NR′—, —O—R″—CH₂—NR′—, —O—CH₂—R″—NR′, —O—R″—CH₂—, —O—CH₂—R″—, —S—R″—CH₂—NR′—, —S—CH₂—R″—NR′, S—R″—CH₂—, —S—CH₂—R″—, —C(═O)NR′—R″—, —C(═O)NR′—R″—NR′—, and the like, and combinations thereof, where each R′ is independently selected from hydrogen, alkyl, alkanyl, alkenyl, alkynyl, aryl, arylalkyl, heteroaryl and heteroarylalkyl, as defined herein, and wherein R″ is a straight or branched alkylene chain of 1 to 40 carbon atoms. One example of a heteroatomic group is —NR′—wherein R′ is hydrogen; another heteroatomic group is a disulfide —S—S—.

“Aryl” means an aromatic carbocyclic moiety such as phenyl or naphthyl (1- or 2-naphthyl).

“Arylalkyl” means an alkyl having at least one alkyl hydrogen atom replaced with an aryl moiety, such as —CH₂-phenyl, —CH═CH-phenyl, —C(CH₃)═CH-phenyl, and the like.

“Heteroaryl” means an aromatic heterocycle ring of 5 to 10 members and having at least one heteroatom selected from nitrogen, oxygen, and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” (also referred to herein as a “heterocyclic ring”) means a 4- to 7-membered monocyclic, or 7- to 10-membered bicyclic, heterocyclic ring, which is either saturated, unsaturated, or aromatic, and which contains from 1 to 4 heteroatoms independently selected from nitrogen, oxygen, and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle, such as —CH₂morpholinyl, and the like.

“Homocycle” (also referred to herein as homocyclic ring or a carbocycle) means a saturated or unsaturated (but not aromatic) carbocyclic ring containing from 3-7 carbon atoms, such as cyclopropane, cyclobutane, cyclopentane, cyclohexane, cycloheptane, cyclohexene, and the like.

“Halogen” means fluoro, chloro, bromo, and iodo.

“Haloalkyl” means an alkyl having at least one hydrogen atom replaced with halogen, such as trifluoromethyl and the like.

“Alkoxy” means an alkyl moiety attached through an oxygen bridge (i.e., —O-alkyl) such as methoxy, ethoxy, and the like.

“Thioalkyl” means an alkyl moiety attached through a sulfur bridge (i.e., —S-alkyl) such as methylthio, ethylthio, and the like.

Linkers (e.g., Z wherein Z=Z¹ or Z²-R⁰ or Z²-R⁰-Z³, and L) are “divalent” in that the linker has two different points of attachment to join or attach one molecule to another molecule. For instance, the linker L may join or attach a Y group to an A2E molecule or a derivative thereof. Generally, two hydrogen atoms have been removed from the linker such that the linker can join or attach to two separate molecules. For example, a “divalent” alkyl means that two hydrogen atoms have been removed from the alkyl such that the alkyl group is attached or linked with two other chemical moieties.

“Pharmaceutically acceptable salt” includes both acid and base addition salts. A pharmaceutically acceptable salt of structures (1), (2), and (3) as well as of substructures thereof is intended to encompass any and all pharmaceutically suitable salt forms. Preferred pharmaceutically acceptable salts of the compounds described herein that have a positive charge on the pyridine nitrogen of A2E-like molecules, A2E derivatives, and A2E immunoconjugates are pharmaceutically acceptable acid addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

It is understood that one of ordinary skill in the art would be able to make the compounds of the invention by methods similar to the methods described herein or by methods known to one of ordinary skill in the art. It is also understood that one of ordinary skill in the art would be able to make in a similar manner as described below other compounds of structures (1), (2), and (3) not specifically illustrated below by using the appropriate starting components and modifying the parameters of the synthesis as needed. In general, compounds employed as initial starting materials in the synthesis of the compounds of the invention are well known and commercially available, e.g., from Sigma Aldrich, Lancaster Synthesis, Inc., Maybridge, Matrix Scientific, TCI, and Fluorochem USA, etc. To the extent that the compounds employed as initial starting materials are not commercially available, the compounds may be readily synthesized using specific references provided, or by standard procedures commonly employed by those of ordinary skill in the art and/or found in general references text (see, for example, Comprehensive Organic Transformations, VCH Publishers Inc., 1989; Compendium of Organic Synthetic Methods, Volumes 1-10, 1974-2002, Wiley Interscience; Advanced Organic Chemistry: Reactions, Mechanisms, and Structure, 5th edition, Wiley Interscience, 2001; Advanced Organic Chemistry, 4th Edition, Part B, Reactions and Synthesis, Kluwer Academic/Plenum Publishers, 2000, etc., and references cited therein).

A2E derivatives that can be used for conjugating (attaching or joining) to a carrier molecule, such as a hapten peptide or carrier polypeptide, may be prepared according to methods described herein. An A2E derivative that has a linker at the nitrogen atom of the pyridyl ring preserves the original geometry of the molecule as well as maintains the A2E molecule's positive charge. Such an A2E derivative (which is a compound having structure (3)) can be made by reacting two molecules of all-trans-retinal (structure 4) with one molecule of a suitably protected amine (structure 5) according to a procedure for making A2E (Parish et al., Proc. Natl. Acad. Sci. USA 95:14609-13 (1998)) followed by acidic deprotection. The reaction scheme for such a method is depicted as Reaction Scheme 1 as follows.

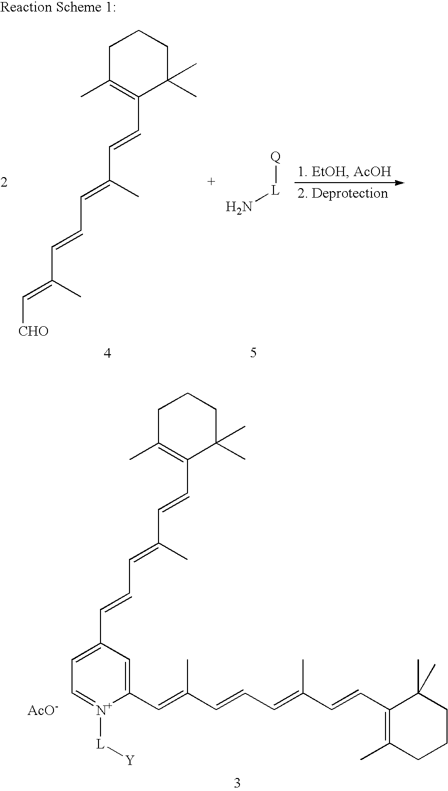

Alternatively, the A2E derivative compound of structure (3) may be prepared according to a method depicted in Reaction Scheme 2. A pyridine precursor (compound 6) is alkylated according to a method described in Ren et al. (J. Am. Chem. Soc. (Communication) 119:3619-20 (1997)) with a suitably protected linker (compound 7). The reaction scheme is exemplified as follows in Reaction Scheme 2.

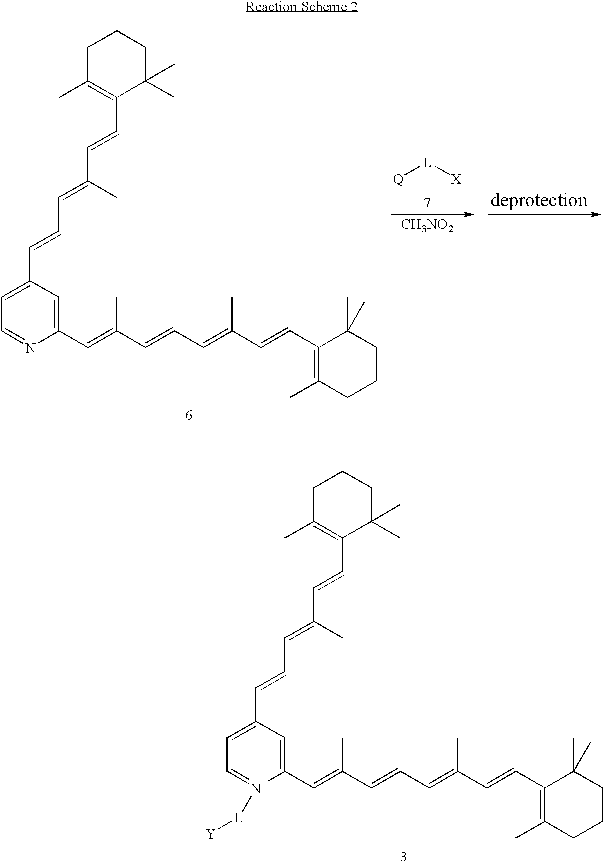

In both Reaction Scheme 1 and 2, “Q” represents a suitably protected electrophilic or nucleophilic group, as described herein. A representative Q group is STr, —C(═O)OH, —C(═O)OR, —NHBoc, —NHC(═O)CF₃, —NHFmoc, or —CONHNHBoc, wherein R is an alkyl. Tr (trityl or triphenylmethyl), Fmoc, and Boc (tert-butoxycarbonyl) are blocking groups known and used by persons skilled in the art for protecting a reactive group to prevent formation of undesired products via an active site. As described in detail herein, L is a divalent linker group -R₁-, -R₂-, or -R₃-, and R₁ is divalent C₁-C₆ alkyl; R₂ is C₁-C₄₀ alkylene or C₁-C₄₀ heteroalkylene; and R₃ is a polyethylene glycol having the formula —(CH₂CH₂O)ₙCH₂CH₂— wherein n=2-12; Y is an electrophilic or nucleophilic moiety suitable for reaction of the compound with an amino acid. X is a leaving group and can include a halogen (bromo (Br), chloro (Cl) or iodo (I)). “Q” represents a suitably protected electrophilic or nucleophilic group, which after deprotection, can be reacted with a carrier polypeptide to form an A2E immunoconjugate according to methods described herein and with which a skilled artisan is familiar.

According to an alternative method, an A2E derivative can be linked to the carrier polypeptide via one of the β-ionone rings, or the A2E molecule can be linked to the unsaturated chains, in a manner that preserves the geometry of the pyridinium cation. The following reaction schemes illustrate reaction schemes for obtaining the A2E like compounds having structure 1 or structure 2 or any of the substructures and specific structures defined herein (see Reaction Schemes 3-7). For instance, the reaction schemes depict methods for obtaining compounds having structure 1 or structure 2, wherein J is -Z¹-Y, -Z²-R⁰—Y, -Z²-R⁰-Z³-Y, or -Z²-Y′—R⁰, and wherein Z¹ is a divalent C₁-C₄₀ alkyl, Z² is a divalent C₁-C₄₀ alkyl, and R⁰ is a monovalent or divalent optionally substituted homocycle, aryl, heteroaryl, or heterocycle; and wherein Y is a monovalent electrophilic or nucleophilic moiety suitable for covalent attachment of the compound to an amino acid, and wherein Y′ is a divalent electrophilic or nucleophilic moiety suitable for covalent attachment of the compound to an amino acid. In certain embodiments, Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl. In another specific embodiment, Y is —OH, —SH, —NH₂, —C(═O)OH, or oxo; in a particular specific embodiment, Y is —NH₂.

For example, in Reaction Scheme 3, the pyridine precursor of A2E (compound 6) can be oxidized by manganese dioxide to form a mixture of ketones (isomers represented by compound 8), which can then be alkylated with iodoethanol in nitromethane to generate hydroxyethylpyridinium derivatives (represented by compound 9) (see Ren et al., supra). The A2E derivative(s) can then be conjugated (attached) to a carrier polypeptide according to methods described herein and practiced in the art (see, e.g., Pestka et al., J. Food Prot. 48: 953-57 (1985)). The Reaction Scheme 3 for making such an A2E derivative and the A2E immunoconjugate (A2E-carrier polypeptide conjugate) is provided as follows.

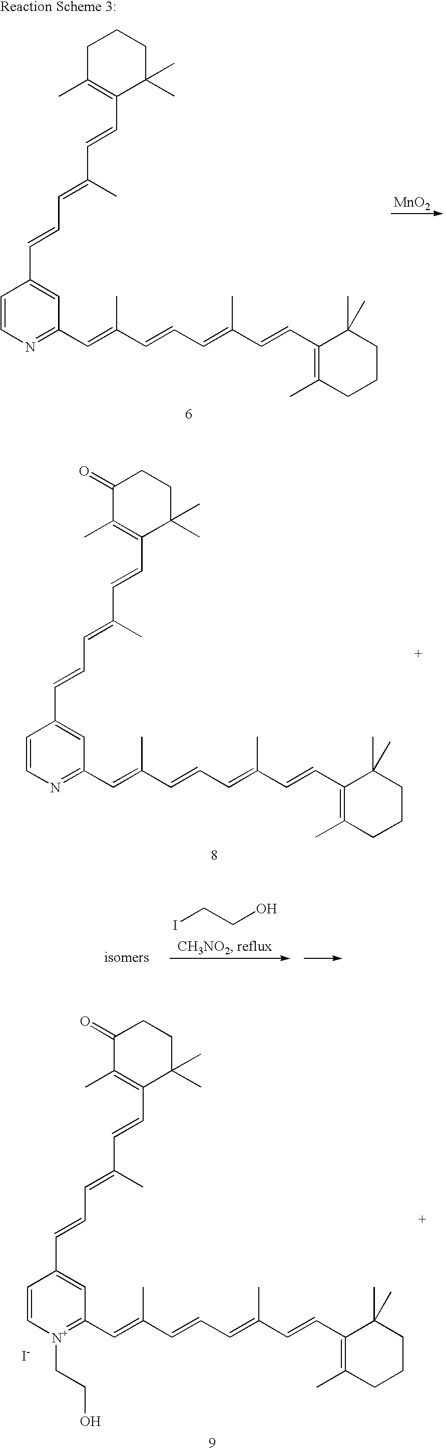

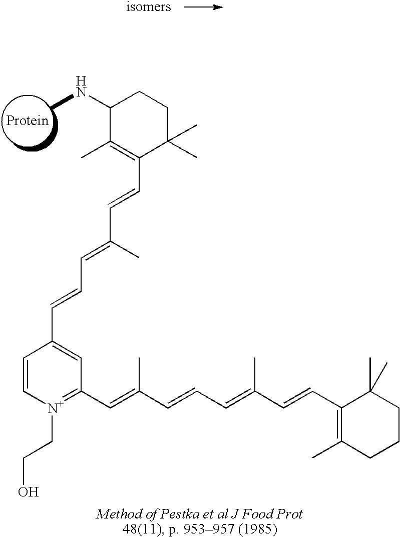

In another embodiment, an A2E derivative may be made by introducing an amino group, as well as other nucleophilic or electrophilic groups, into the A2E molecule according to the following method. A compound of structure (10) may be prepared according to the method described in Tanaka et al. (J. Org. Chem. 66: 3099-3110 (2001)). Oxidation of this compound (structure 10) by manganese dioxide leads to the corresponding aldehyde compound of structure (11). The method includes a Wittig reaction of this aldehyde (structure 11) with a phosphonium salt (structure 12), deprotection with tetrabutylammonium fluoride (TBAF) in tetrahydrofuran (THF) (see Reaction Scheme 4). (TBDMS refers to t-butyldimethylsilyl) and P+Ph₃Br refers to triphenylphosphonium bromide.) Oxidation of the resulting alcohol results in formation of the aldehyde of structure (14). A compound having structure (12) may be preparing according to methods practiced in the art. For example, a method for synthesis of such a compound (structure (12)) is described in Curley et al. (J. Org. Chem. 49: 1941-44 (1984)). Following the second Wittig coupling, the amino derivative of A2E (compound of structure (17)) is formed by alkylation of the pyridine nitrogen with iodoethanol in nitromethane and removal of the t-butyloxycarbonyl group with trifluoroacetic acid (TFA). This amino derivative (A2E derivative of structure (17)) can be linked to a carrier polypeptide via glutaraldehyde crosslinking or other chemistries known to a person skilled in the art.

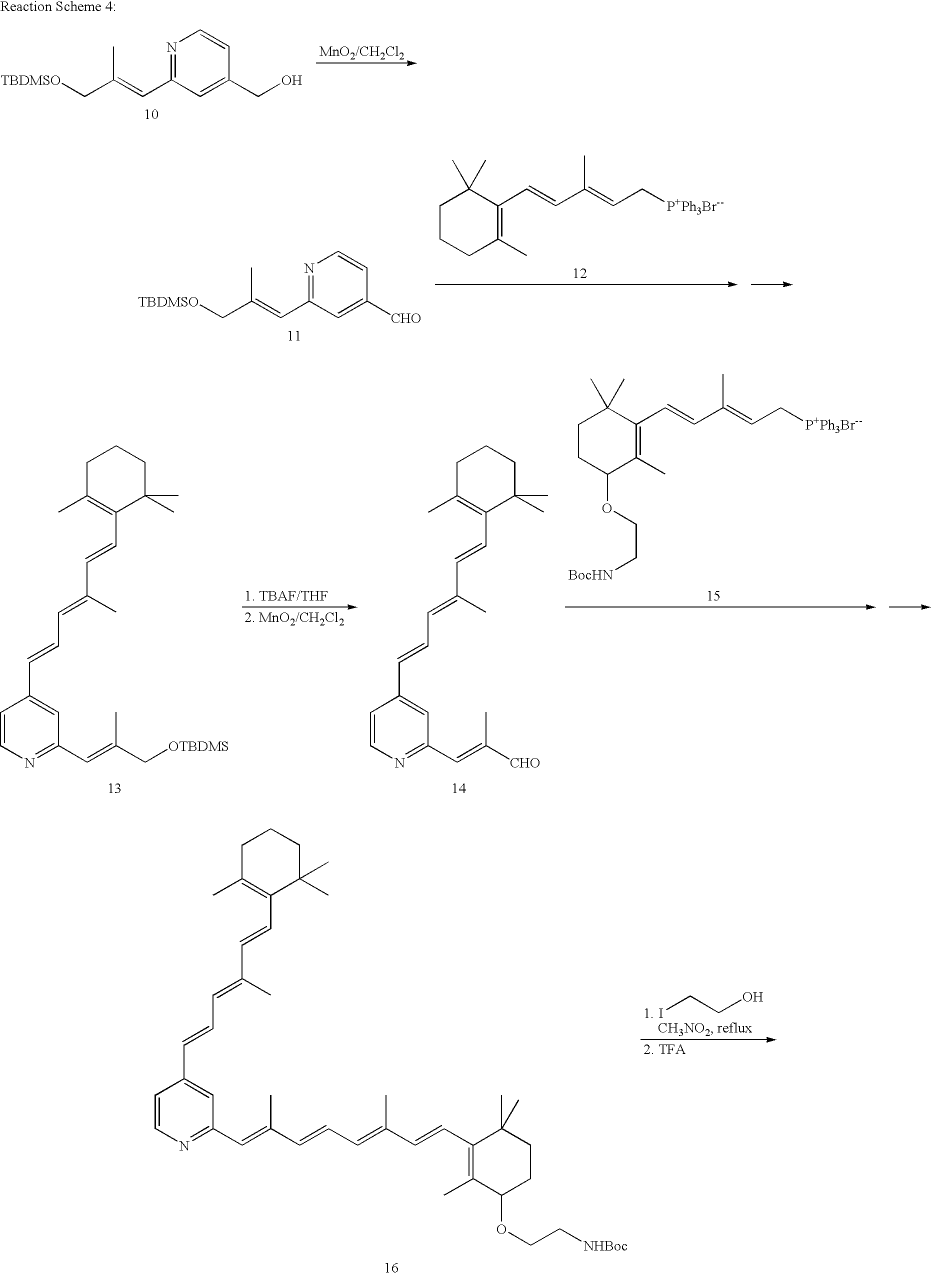

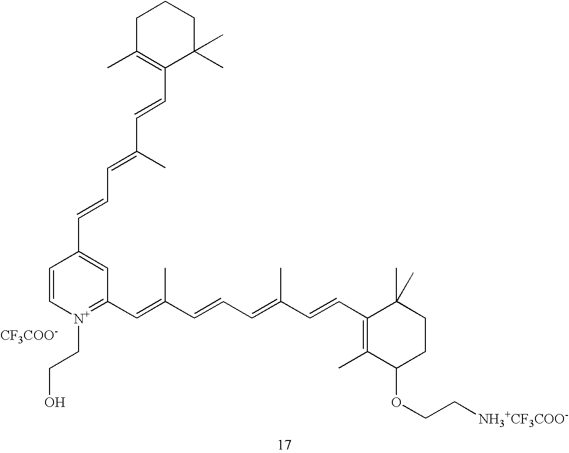

A mixture of A2E derivatives that are useful for conjugating to a carrier molecule such as a carrier polypeptide can be made by first preparing a derivative of retinal that bears a suitably protected reactive group, for example, a diethyl acetal of 4-bromo-retinal compound of structure (19) (see, e.g., Chapman et al., J. Biochem. Biophys. Meth. 9:287-300 (1989)). Reaction of a compound of structure (19) with Boc-protected 2-aminoethane thiol (compound of structure (20)) followed by mild deprotection of the acetal (compound of structure (21)) using oxalic acid leads to the retinal derivative compound of structure (22). This derivative is converted into the Schiff base (compound of structure (23)) by reacting it with ethanolamine, and the Schiff base can be condensed with retinal to form a compound of structure (24) that is a mixture of isomers. (p-TsOH refers to p-toluenesulfonic acid, and NBS refers to N-bromosuccinimide.) Because formation of Schiff bases is a reversible process, formation of the mixture of isomers of A2E derivatives is expected. The A2E derivatives can be deprotected and the isomers separated either by flash chromatography or high performance liquid chromatography or other methods practiced in the art, and then one or more isomers may be conjugated to a carrier polypeptide. Alternatively, the A2E derivatives can be conjugated to the carrier polypeptide without separation of the isomers. The reaction is presented in the following schematic (Reaction Scheme 5).

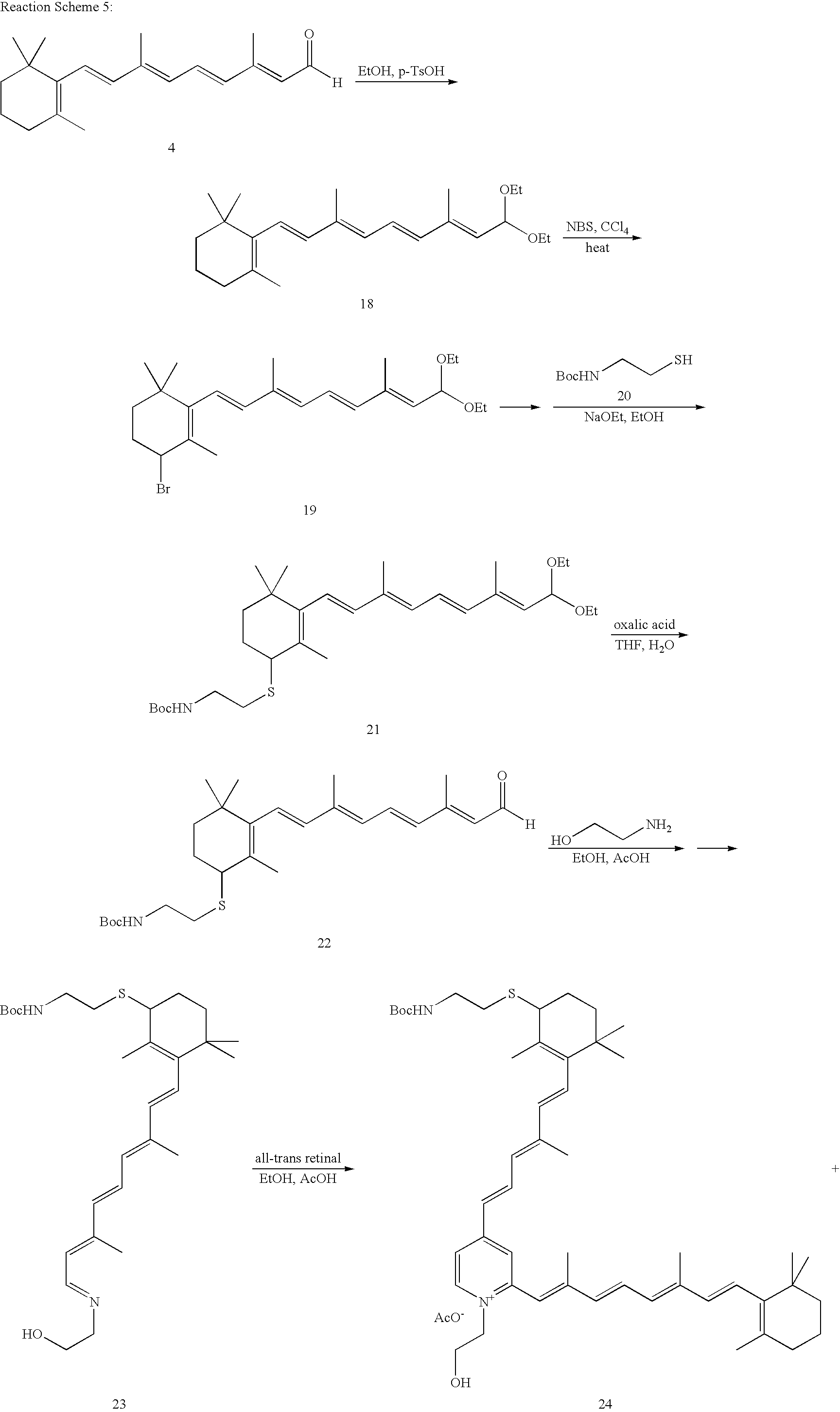

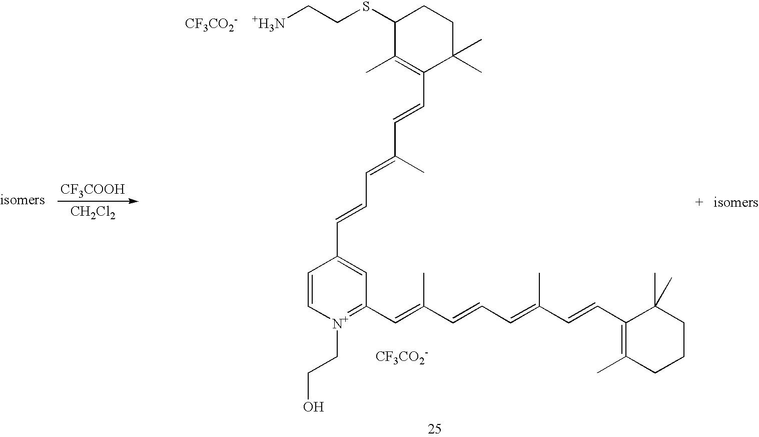

Another exemplary method for making an A2E immunoconjugate involves making an A2E derivative that has the entire unsaturated chain of the A2E compound replaced with another moiety or that has a portion of an unsaturated chain of the A2E molecule replaced with another moiety. For example, a portion of the unsaturated chain may be the entire chain except for the portion of the chain comprising the double bond adjacent to the pyridine ring, the retention of which, without wishing to be bound by theory, may minimize impact on the electronic properties of the ring. Such an A2E derivative would have at least one epitope that includes one β-ionone ring and the 2-hydroxypyridinium moiety. An antibody that specifically binds to such an epitope that comprises the unique A2E features of a β-ionone ring and the 2-hydroxypyridinium moiety is expected to bind specifically to A2E and A2E isomers and not to cross-react with or recognize other retinal related compounds that lack these features. An example of such an A2E derivative is an amino substituted phenyl analog of A2E that may be prepared according to the synthesis method represented in the following Reaction Scheme 6.

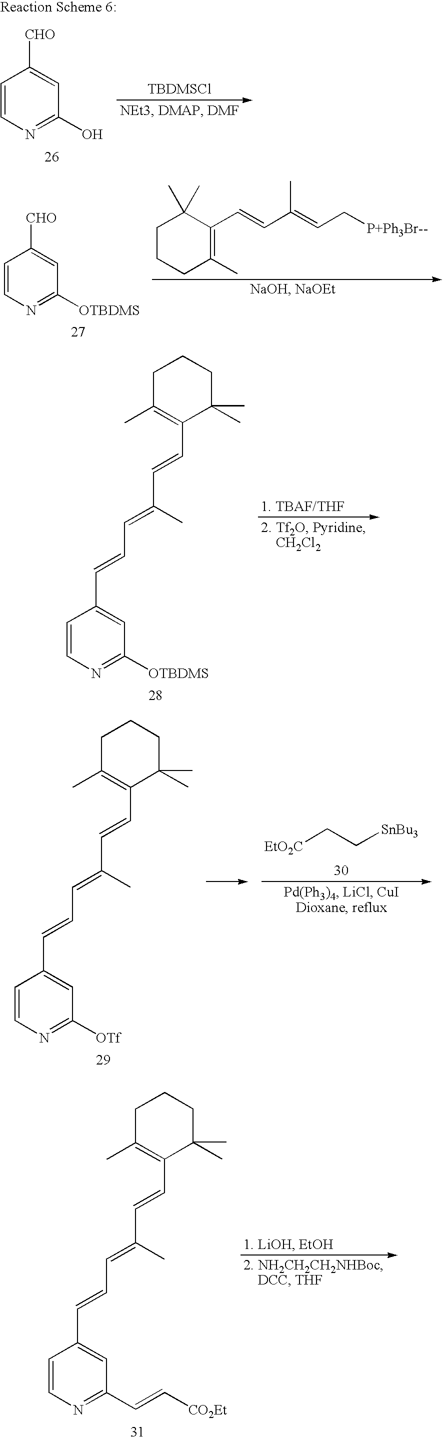

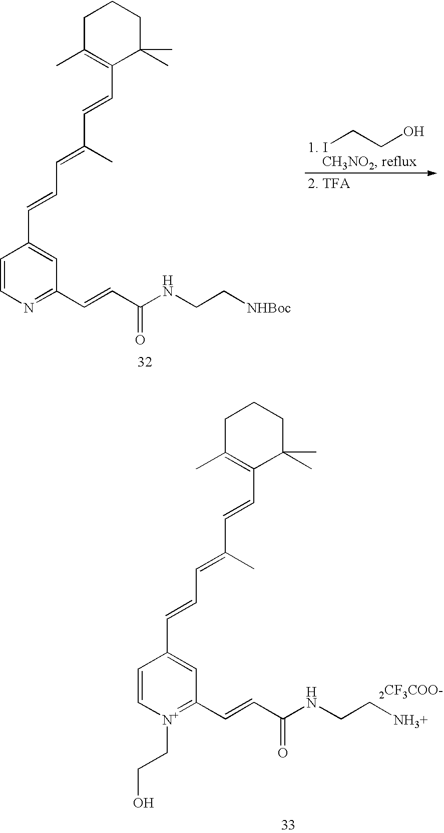

In the method depicted by Reaction Scheme 6, the 2-hydroxy pyridine 4-aldehyde (compound of structure (26)) is obtained according to the method described in Ren et al., supra. The compound of structure (26) is protected using t-butyldimethylsilyl chloride (TBDMSCl) and catalytic amounts of 4-dimethylaminopyridine (DMAP) to form the TBDMS derivative (compound of structure (27)), which structure is then coupled through a Wittig reaction with the phosphonium bromide derivative (compound of structure (12) according to methods practiced in the art to form the compound of structure (28). This compound of structure (28) is deprotected using tetrabutylammonium fluoride (TBAF) in tetrahydrofuran (THF) and converted to the triflate compound of structure (29) by reaction of the deprotected alcohol intermediate with triflic anhydride in pyridine and dichloromethane. The triflate compound of structure (29) undergoes palladium-catalyzed Stille coupling with the tin reagent of structure (30) (which reagent is prepared according methods practiced in the art) to form the compound having structure (31), which is converted to a compound of structure (32) through the hydrolysis of the ethyl ester followed by coupling of the intermediate acid with mono-Boc ethanolamine. The pyridine nitrogen of compound of structure (32) is alkylated with iodoethanol in nitromethane in a similar manner as described in Ren et al., supra, and the amino group is deprotected using trifluoroacetic acid to form the A2E derivative compound of structure (33). This compound (structure 33) may be conjugated to a carrier polypeptide, for example, BSA, using the glutaraldehyde method according to procedures practiced in the art or by using other methods known in the art for conjugating an amine to a carrier polypeptide (see, e.g., Pierce Biotechnology Catalog (2002)).

According to another method for making A2E derivatives, A2E can be converted into a mixture of epoxides. A2E is converted into a mixture of epoxides (compounds of structure 34) by treating A2E with peroxy organic acids such as MCPBA (meta-chloroperbenzoic acid) or by exposure to a blue light as described in Sparrow et al. (J. Biol. Chem. 278(20): 18207-213 (2003)). The resulting epoxides (compounds 34) are combined and reacted with a carrier polypeptide directly to yield A2E-carrier polypeptide conjugates (compounds of structure (35)). The method is represented by the following Reaction Scheme 7.

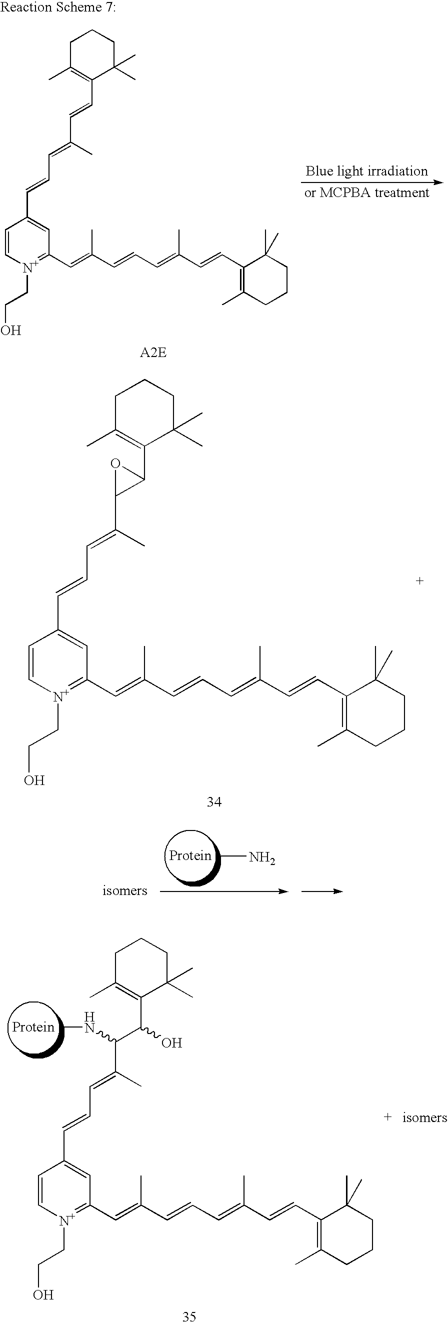

In general, the compounds used in the reactions described herein may be made according to organic synthesis techniques known to those skilled in this art, starting from commercially available chemicals and/or from compounds described in the chemical literature. “Commercially available chemicals” may be obtained from standard commercial sources including Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), and Wako Chemicals USA, Inc. (Richmond Va.).

Methods known to one of ordinary skill in the art may be identified through various reference books and databases. Suitable reference books and treatise that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds of the present invention, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present invention is P. H. Stahl & C. G. Wermuth “Handbook of Pharmaceutical Salts”, Verlag Helvetica Chimica Acta, Zurich, 2002.

Antibodies Specific for A2E

Provided herein are antibodies that specifically bind to A2E and methods of making and using these antibodies. A2E specific antibodies may be polyclonal or monoclonal, prepared by immunization of animals and isolation of the antibody, or the antibody may be a recombinant antibody. The A2E immunoconjugates (i.e., A2E or a an A2E derivative thereof as described in detail herein conjugated or attached to an amino acid of a carrier molecule such as a hapten peptide or a carrier polypeptide or a hapten peptide attached or conjugated to a carrier polypeptide) described herein may be used for immunizing an animal to generate a humoral immune response that results in production of antibodies that specifically bind to A2E. Such antibodies may also be useful as reagents for immunochemical analyses to detect the presence of A2E in a biological sample. Antibodies that specifically bind to A2E may also be used to as therapeutic agents that decrease, inhibit, reduce, or prevent accumulation of A2E in or near retinal neuronal cells and thereby reduce, abrogate, prevent, or decrease (decrease in a statistically or biologically significant manner) deleterious or toxic effects that result from accumulation of A2E in the eye. The antibodies that specifically bind to A2E in the eye (e.g. at the RPE) may be used to treat a subject that has an ophthalmic disease or disorder (or who may be at risk for developing an ophthalmic disease or disorder) and may be used to inhibit degeneration of a retinal cell (such as a retinal neuronal cell), enhance retinal cell survival and/or increase retinal cell viability, which prevents, inhibits, slows the progression of, or reduces the symptoms associated with, an ophthalmic disease. Such antibodies that may be used for treatment or prevention of an ophthalmic disease preferably do not cross-react with other retinoid related compounds that are present in a normal eye, such as all-trans-retinal, retinyl palmitate, β-carotene, 11-cis-retinal, and 13-cis retinoic acid. Ophthalmic diseases and disorders are described in detail herein and include, but are not limited to, macular degeneration, glaucoma, diabetic retinopathy, retinal detachment, retinal blood vessel occlusion, retinitis pigmentosa, optic neuropathy, inflammatory retinal disease, a retinal disorder associated with Alzheimer's disease, and a retinal disorder associated with multiple sclerosis. In a particular embodiment the ophthalmic disease is macular degeneration.

An ophthalmic disease or disorder may result, at least in part, from lipofuscin pigment accumulation and/or from accumulation of A2E in the eye. An antibody that specifically binds to A2E may inhibit (i.e., prevent, reduce, slow, abrogate, minimize) A2E accumulation in the eye, and may inhibit an accumulation of a lipofuscin pigment in the eye. Accordingly, in certain embodiments, methods are provided for inhibiting or preventing accumulation of lipofuscin pigment and/or A2E in the eye of a subject.

A2E immunoconjugates or A2E may also be used in methods for screening samples containing antibodies, for example, samples of purified antibodies, antisera, or cell culture supernatants, or any other biological sample that may contain one or more antibodies specific for A2E. A2E immunoconjugates or A2E may also be used in methods for identifying and selecting from a biological sample one or more B cells that are producing an antibody that specifically binds to A2E (e.g., plaque forming assays and the like). The B cells may then be used as source of an A2E specific antibody-encoding polynucleotide that can be cloned and/or modified by recombinant molecular biology techniques known in the art and described herein.

As used herein, an antibody is said to be “immunospecific” or to “specifically bind” A2E if it reacts at a detectable level with A2E, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹, and that do not specifically bind to another related retinoid molecule. Affinity of an antibody for its cognate antigen is also commonly expressed as a dissociation constant K_{D}, and an anti-A2E antibody specifically binds to A2E if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M.

Affinities of binding partners or antibodies can be readily determined using conventional techniques, for example, those described by Scatchard et al. (Ann. N.Y. Acad. Sci. USA 51:660 (1949)) and by surface plasmon resonance (SPR; BIAcore™, Biosensor, Piscataway, N.J.). For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the surface plasmon resonance signal can be analyzed to yield apparent rate constants for the association and dissociation phases of the binding reaction. The ratio of these values gives the apparent equilibrium constant (affinity) (see, e.g., Wolff et al., Cancer Res. 53:2560-2565 (1993).

Binding properties of an antibody to A2E may generally be determined and assessed using immunodetection methods including, for example, an enzyme-linked immunosorbent assay (ELISA), immunoprecipitation, immunoblotting, countercurrent immunoelectrophoresis, radioimmunoassays, dot blot assays, inhibition or competition assays, and the like, which may be readily performed by those having ordinary skill in the art (see, e.g., U.S. Pat. Nos. 4,376,110 and 4,486,530; Harlow et al., Antibodies: A Laboratory Manual, Cold Spring Harbor Laboratory (1988)). Immunoassay methods may include controls and procedures to determine whether antibodies bind specifically to A2E and do not recognize or cross-react with other retinoid compounds, such as all-trans-retinal, all-trans-retinoic acid, retinyl palmitate, β-carotene, and 13-cis retinoic acid, and the like, that share some similar structural features with A2E but which do not comprise the unique pyridyl ring described herein. In addition, an immunoassay performed for detection of anti-A2E antibodies that are produced in response to immunization of a host with an A2E conjugated to a particular carrier polypeptide may incorporate the use of A2E that is conjugated to a different carrier polypeptide than that used for immunization to reduce or eliminate detection of antibodies that bind specifically to the immunizing carrier polypeptide. Alternatively, A2E or an A2E derivative or an A2E like compound as described herein that is not conjugated to a carrier molecule may be used in an immunoassay for detecting anti-A2E specific antibodies.

Antibodies may generally be prepared by any of a variety of techniques known to those having ordinary skill in the art. See, e.g., Harlow et al., Antibodies: A Laboratory Manual, Cold Spring Harbor Laboratory (1988). Any one of the A2E immunoconjugates described herein or A2E or a cell expressing A2E may be used as an immunogen for immunizing an animal for production of either polyclonal antibodies or monoclonal antibodies. An antibody according to the present invention may belong to any immunoglobulin class, for example IgG, IgE, IgM, IgD, or IgA. It may be obtained from or derived from an animal, for example, fowl (e.g., chicken) and mammals, which include but are not limited to a mouse, rat, hamster, rabbit, or other rodent, a cow, horse, sheep, goat, camel, human, or other primate. The antibody may be an internalising antibody. In one such technique, an animal is immunized with A2E or an A2E immunoconjugate as described herein as an antigen to generate polyclonal antisera. Suitable animals include, for example, rabbits, sheep, goats, pigs, cattle, and may also include smaller mammalian species, such as mice, rats, and hamsters, or other species.

Polyclonal antibodies that bind specifically to A2E can be prepared using methods described herein and practiced by persons skilled in the art (see, for example, Green et al., “Production of Polyclonal Antisera,” in Immunochemical Protocols (Manson, ed.), pages 1-5 (Humana Press 1992); Harlow et al., Antibodies: A Laboratory Manual, Cold Spring Harbor Laboratory (1988); Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995)). Although polyclonal antibodies are typically raised in animals such as rats, mice, rabbits, goats, cattle, or sheep, an anti-A2E antibody may also be obtained from a subhuman primate. General techniques for raising diagnostically and therapeutically useful antibodies in baboons may be found, for example, in WO 91/11465 (1991) and in Losman et al., Int. J. Cancer 46:310, 1990.

In addition, the A2E immunoconjugate, A2E or derivative thereof, or cell expressing A2E to be used as immunogen may be emulsified in an adjuvant. See, e.g., Harlow et al., Antibodies: A Laboratory Manual, Cold Spring Harbor Laboratory (1988). Adjuvants typically used for immunization of non-human animals include but are not limited to Freund's complete adjuvant, Freund's incomplete adjuvant, montanide ISA, Ribi Adjuvant System (RAS), nitrocellulose-adsorbed antigen. The immunogen may be injected into the animal via any number of different routes, including intraperitoneally, intravenousely, intramuscularly, intraocularly, intradermally, or subcutaneously. In general, after the first injection, animals receive one or more booster immunizations according to a preferred schedule that may vary according to, inter alia, the antigen, the adjuvant (if any) and/or the particular animal species. The immune response may be monitored by periodically bleeding the animal, separating the sera out of the collected blood, and analyzing the sera in an immunoassay, such as an ELISA or Ouchterlony diffusion assay, or the like, to determine the specific antibody titer. Once an adequate antibody titer is established, the animals may be bled periodically to accumulate the polyclonal antisera. Polyclonal antibodies that bind specifically to A2E may then be purified from such antisera, for example, by affinity chromatography using protein A or protein G immobilized on a suitable solid support (see, e.g., Coligan, supra, p. 2.7.1-2.7.12; 2.9.1-2.9.3; Baines et al., Purification of Immunoglobulin G (IgG), in Methods in Molecular Biology, 10:9-104 (The Humana Press, Inc. (1992)). Alternatively, affinity chromatography may be performed wherein an A2E immunoconjugate or an antibody specific for an Ig constant region of the particular immunized animal species is immobilized on a suitable solid support.

Monoclonal antibodies that specifically bind to A2E and hybridomas, which are examples of immortal eukaryotic cell lines, that produce monoclonal antibodies having the desired binding specificity, may also be prepared, for example, using the technique of Kohler and Milstein (Nature, 256:495-497; 1976, Eur. J. Immunol. 6:511-519 (1975)) and improvements thereto (see, e.g, Coligan et al. (eds.), Current Protocols in Immunology, 1:2.5.1-2.6.7 (John Wiley & Sons 1991); U.S. Pat. Nos. 4,902,614, 4,543,439, and 4,411,993; Monoclonal Antibodies, Hybridomas: A New Dimension in Biological Analyses, Plenum Press, Kennett et al. (eds.) (1980); and Antibodies: A Laboratory Manual, Harlow and Lane (eds.), Cold Spring Harbor Laboratory Press (1988); see also, e.g., Brand et al., Planta Med. 70:986-92 (2004)). An animal—for example, a rat, hamster, or preferably mouse—is immunized with an A2E immunogen prepared as described above. The presence of specific antibody production may be monitored after the initial injection (injections may be administered by any one of several routes as described herein for generation of polyclonal antibodies) and/or after a booster injection by obtaining a serum sample and detecting the presence of an antibody that binds to A2E using any one of several immunodetection methods known in the art and described herein. From animals producing antibodies that bind to A2E, lymphoid cells, most commonly cells from the spleen or lymph node, are removed to obtain B-lymphocytes, lymphoid cells that are antibody-forming cells. The lymphoid cells, typically spleen cells, may be immortalized by fusion with a drug-sensitized myeloma (e.g., plasmacytoma) cell fusion partner, preferably one that is syngeneic with the immunized animal and that optionally has other desirable properties (e.g., inability to express endogenous Ig gene products, e.g., P3X63-Ag 8.653 (ATCC No. CRL 1580); NS0, SP20)). The lymphoid cells and the myeloma cells may be combined for a few minutes with a membrane fusion-promoting agent, such as polyethylene glycol or a nonionic detergent, and then plated at low density on a selective medium that supports the growth of hybridoma cells, but not unfused myeloma cells. A preferred selection media is HAT (hypoxanthine, aminopterin, thymidine). After a sufficient time, usually about one to two weeks, colonies of cells are observed. Antibodies produced by the cells may be tested for binding activity to A2E. The hybridomas are cloned (e.g., by limited dilution cloning or by soft agar plaque isolation) and positive clones that produce an antibody specific to the antigen are selected and cultured. Hybridomas producing monoclonal antibodies with high affinity and specificity for A2E are preferred.

Monoclonal antibodies may be isolated from the supernatants of hybridoma cultures. An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Contaminants may be removed from the subsequently harvested ascites fluid (usually within 1-3 weeks) by conventional techniques, such as chromatography (e.g., size-exclusion, ion-exchange), gel filtration, precipitation, extraction, or the like (see, e.g., Coligan, supra, p. 2.7.1-2.7.12; 2.9.1-2.9.3; Baines et al., Purification of Immunoglobulin G (IgG), in Methods in Molecular Biology, 10:9-104 (The Humana Press, Inc. (1992)). For example, antibodies may be purified by affinity chromatography using an appropriate ligand selected based on particular properties of the monoclonal antibody (e.g., heavy or light chain isotype, binding specificity, etc.). Examples of a suitable ligand, immobilized on a solid support, include Protein A, Protein G, an anti-constant region (light chain or heavy chain) antibody, an anti-idiotype antibody, an A2E immunoconjugate, and A2E or derivative thereof.

An anti-A2E antibody may be a human monoclonal antibody. Human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein.

For example, human monoclonal antibodies may be obtained from transgenic mice that have been engineered to produce specific human antibodies in response to antigenic challenge. Methods for obtaining human antibodies from transgenic mice are described, for example, by Green et al., Nature Genet. 7:13, 1994; Lonberg et al., Nature 368:856, 1994; Taylor et al., Int. Immun. 6:579, 1994; U.S. Pat. No. 5,877,397; Bruggemann et al., 1997 Curr. Opin. Biotechnol. 8:455-58; Jakobovits et al., 1995 Ann. N.Y. Acad. Sci. 764:525-35. In this technique, elements of the human heavy and light chain locus are artificially introduced by genetic engineering into strains of mice derived from embryonic stem cell lines that contain targeted disruptions of the endogenous murine heavy chain and light chain loci. (See also Bruggemann et al., Curr. Opin. Biotechnol. 8:455-58 (1997)). For example, human immunoglobulin transgenes may be mini-gene constructs, or transloci on yeast artificial chromosomes, which undergo B cell-specific DNA rearrangement and hypermutation in the mouse lymphoid tissue. Human monoclonal antibodies may be obtained by immunizing the transgenic mice, which may then produce human antibodies specific for the antigen. Lymphoid cells of the immunized transgenic mice can be used to produce human antibody-secreting hybridomas according to the methods described herein. Polyclonal sera containing human antibodies may also be obtained from the blood of the immunized animals.

Another method for generating human A2E specific monoclonal antibodies includes immortalizing human peripheral blood cells by EBV transformation. See, e.g., U.S. Pat. No. 4,464,456. Such an immortalized B cell line (or lymphoblastoid cell line) producing a monoclonal antibody that specifically binds to A2E can be identified by immunodetection methods as provided herein, for example, an ELISA, and then isolated by standard cloning techniques. The stability of the lymphoblastoid cell line producing an anti-A2E antibody may be improved by fusing the transformed cell line with a murine myeloma to produce a mouse-human hybrid cell line according to methods known in the art (see, e.g., Glasky et al., Hybridoma 8:377-89 (1989)). Still another method to generate human monoclonal antibodies is in vitro immunization, which includes priming human splenic B cells with antigen, followed by fusion of primed B cells with a heterohybrid fusion partner. See, e.g., Boerner et al., 1991 J. Immunol. 147:86-95.

In certain embodiments, a B cell that is producing an anti-A2E antibody is selected, and the light chain and heavy chain variable regions are cloned from the B cell according to molecular biology techniques known in the art (WO 92/02551; U.S. Pat. No. 5,627,052; Babcook et al., Proc. Natl. Acad. Sci. USA 93:7843-48 (1996)) and described herein. Preferably B cells from an immunized animal are isolated from the spleen, lymph node, or peripheral blood sample by selecting a cell that is producing an antibody that specifically binds to A2E. B cells may also be isolated from humans, for example, from a peripheral blood sample. Methods for detecting single B cells that are producing an antibody with the desired specificity are well known in the art, for example, by plaque formation, fluorescence-activated cell sorting, in vitro stimulation followed by detection of specific antibody, and the like. Methods for selection of specific antibody producing B cells include, for example, preparing a single cell suspension of B cells in soft agar that contains A2E or derivative thereof or an A2E immunoconjugate as described herein. Binding of the specific antibody produced by the B cell to the antigen results in the formation of a complex, which may be visible as an immunoprecipitate. After the B cells producing the specific antibody are selected, the specific antibody genes may be cloned by isolating and amplifying DNA or mRNA according to methods known in the art and described herein.

Chimeric antibodies, specific for A2E, including humanized antibodies, may also be generated according to the present invention. A chimeric antibody has at least one constant region domain derived from a first mammalian species and at least one variable region domain derived from a second, distinct mammalian species. See, e.g., Morrison et al., Proc. Natl. Acad. Sci. USA, 81:6851-55 (1984). In one embodiment, a chimeric antibody may be constructed by cloning the polynucleotide sequence that encodes at least one variable region domain derived from a non-human monoclonal antibody, such as the variable region derived from a murine, rat, or hamster monoclonal antibody, into a vector containing a nucleic acid sequence that encodes at least one human constant region (see, e.g., Shin et al., Methods Enzymol. 178:459-76 (1989); Walls et al., Nucleic Acids Res. 21:2921-29 (1993)). By way of example, the polynucleotide sequence encoding the light chain variable region of a murine monoclonal antibody may be inserted into a vector containing a nucleic acid sequence encoding the human kappa light chain constant region sequence. In a separate vector, the polynucleotide sequence encoding the heavy chain variable region of the monoclonal antibody may be cloned in frame with sequences encoding the human IgG1 constant region. The particular human constant region selected may depend upon the effector functions desired for the particular antibody (e.g., complement fixing, binding to a particular Fc receptor, etc.). Another method known in the art for generating chimeric antibodies is homologous recombination (e.g., U.S. Pat. No. 5,482,856). Preferably, the vectors will be transfected into eukaryotic cells for stable expression of the chimeric antibody.

A non-human/human chimeric antibody may be further genetically engineered to create a “humanized” antibody. Such a humanized antibody may comprise a plurality of CDRs derived from an immunoglobulin of a non-human mammalian species, at least one human variable framework region, and at least one human immunoglobulin constant region. Humanization may in certain embodiments provide an antibody that has decreased binding affinity for A2E when compared, for example, with either a non-human monoclonal antibody from which an A2E binding variable region is obtained, or a chimeric antibody having such a V region and at least one human C region, as described above. Useful strategies for designing humanized antibodies may therefore include, for example by way of illustration and not limitation, identification of human variable framework regions that are most homologous to the non-human framework regions of the chimeric antibody. Without wishing to be bound by theory, such a strategy may increase the likelihood that the humanized antibody will retain specific binding affinity for A2E, which in some preferred embodiments may be substantially the same affinity for A2E, and in certain other embodiments may be a greater affinity for A2E (see, e.g., Jones et al., Nature 321:522-25 (1986); Riechmann et al., Nature 332:323-27 (1988)).

Designing such a humanized antibody may therefore include determining CDR loop conformations and structural determinants of the non-human variable regions, for example, by computer modeling, and then comparing the CDR loops and determinants to known human CDR loop structures and determinants (see, e.g., Padlan et al., FASEB 9:133-39 (1995); Chothia et al., Nature, 342:377-383 (1989)). Computer modeling may also be used to compare human structural templates selected by sequence homology with the non-human variable regions (see, e.g., Bajorath et al., Ther. Immunol. 2:95-103 (1995); EP-0578515-A3). If humanization of the non-human CDRs results in a decrease in binding affinity, computer modeling may aid in identifying specific amino acid residues that could be changed by site-directed or other mutagenesis techniques to partially, completely or supra-optimally (i.e., increase to a level greater than that of the non-humanized antibody) restore affinity. Those having ordinary skill in the art are familiar with these techniques, and will readily appreciate numerous variations and modifications to such design strategies.

One such method for preparing a humanized antibody is called veneering. Veneering framework (FR) residues refers to the selective replacement of FR residues from, e.g., a rodent heavy or light chain V region, with human FR residues in order to provide a xenogeneic molecule comprising an antigen-binding site that retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface (see, e.g, Davies et al., Ann. Rev. Biochem. 59:439-73, (1990)). Thus, antigen binding specificity can be preserved in a humanized antibody when the CDR structures, their interaction with each other, and their interaction with the rest of the V region domains are carefully maintained. By using veneering techniques, exterior (e.g., solvent-accessible) FR residues that are readily encountered by the immune system are selectively replaced with human residues to provide a hybrid molecule that comprises either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR that differ from the human counterpart are replaced by the residues present in the human moiety using recombinant techniques well known in the art. Residue switching is only carried out with moieties that are at least partially exposed (solvent accessible), and care is exercised in the replacement of amino acid residues that may have a significant effect on the tertiary structure of V region domains, such as proline, glycine, and charged amino acids.

In this manner, the resultant “veneered” antigen-binding sites are designed to retain the rodent CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs that are believed to influence the “canonical” tertiary structures of the CDR loops (see, e.g., Chothia et al., Nature, 342:377-383 (1989)). These design criteria are then used to prepare recombinant nucleotide sequences that combine the CDRs of both the heavy and light chain of a antigen-binding site into human-appearing FRs that can be used to transfect mammalian cells for the expression of recombinant human antibodies that exhibit the antigen specificity of the rodent antibody molecule.

For particular uses, antigen-binding fragments of A2E antibodies may be desired. Antibody fragments, F(ab′)₂ Fab, Fab′, Fv, and Fd, can be obtained, for example, by proteolytic hydrolysis of the antibody, for example, pepsin or papain digestion of whole antibodies according to conventional methods. As an illustration, antibody fragments can be produced by enzymatic cleavage of antibodies with pepsin to provide a fragment denoted F(ab′)₂. This fragment can be further cleaved using a thiol reducing agent to produce an Fab′ monovalent fragment. Optionally, the cleavage reaction can be performed using a blocking group for the sulfhydryl groups that result from cleavage of disulfide linkages. As an alternative, an enzymatic cleavage of an antibody using papain produces two monovalent Fab fragments and an Fc fragment (see, e.g., U.S. Pat. No. 4,331,647; Nisonoff et al., Arch. Biochem. Biophys. 89:230, 1960; Porter, Biochem. J. 73:119, 1959; Edelman et al., in Methods in Enzymology 1:422 (Academic Press 1967); Weir, Handbook of Experimental Immunology, Blackwell Scientific, Boston (1986)). The antigen binding fragments may be separated from the Fc fragments by affinity chromatography, for example, using immobilized protein A, protein G, an Fc specific antibody, or immobilized A2E or a derivative thereof, or an A2E immunoconjugate. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the A2E molecule that is recognized by the intact antibody.

An antibody fragment may also be any synthetic or genetically engineered protein that acts like an antibody in that it binds to a specific antigen to form a complex. For example, antibody fragments include isolated fragments consisting of the light chain variable region, Fv fragments consisting of the variable regions of the heavy and light chains, recombinant single chain polypeptide molecules in which light and heavy variable regions are connected by a peptide linker (scFv proteins), and minimal recognition units consisting of the amino acid residues that mimic the hypervariable region. The antibody of the present invention preferably comprises at least one variable region domain. The variable region domain may be of any size or amino acid composition and will generally comprise at least one hypervariable amino acid sequence responsible for antigen binding and which is adjacent to or in frame with one or more framework sequences. In general terms, the variable (V) region domain may be any suitable arrangement of immunoglobulin heavy (V_{H}) and/or light (V_{L}) chain variable domains. Thus, for example, the V region domain may be monomeric and be a V_{H} or V_{L} domain, which is capable of independently binding antigen with acceptable affinity. Alternatively, the V region domain may be dimeric and contain V_{H}-V_{H}, V_{H}-V_{L}, or V_{L}-V_{L}, dimers. Preferably, the V region dimer comprises at least one V_{H} and at least one V_{L} chain that are non-covalently associated (hereinafter referred to as Fᵥ). If desired, the chains may be covalently coupled either directly, for example via a disulfide bond between the two variable domains, or through a linker, for example a peptide linker, to form a single chain Fv (scFᵥ).

A minimal recognition unit is an antibody fragment comprising for a single complementarity-determining region (CDR). Such CDR peptides can be obtained by constructing polynucleotides that encode the CDR of an antibody of interest. The polynucleotides are prepared, for example, by using the polymerase chain reaction to synthesize the variable region using mRNA of antibody-producing cells as a template according to methods practiced by persons skilled in the art (see, for example, Larrick et al., Methods: A Companion to Methods in Enzymology 2:106, (1991); Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley-Liss, Inc. 1995)). Alternatively, such CDR peptides and other antibody fragment can be synthesized using an automated peptide synthesizer.

According to certain embodiments, non-human, human, or humanized heavy chain and light chain variable regions of any of the Ig molecules described herein may be constructed as scFv polypeptide fragments (single chain antibodies). See, e.g., Bird et al., Science 242:423-426 (1988); Huston et al., Proc. Natl. Acad. Sci. USA 85:5879-5883 (1988)). Multi-functional scFv fusion proteins may be generated by linking a polynucleotide sequence encoding an scFv polypeptide in-frame with at least one polynucleotide sequence encoding any of a variety of known effector proteins. These methods are known in the art, and are disclosed, for example, in EP-B1-0318554, U.S. Pat. No. 5,132,405, U.S. Pat. No. 5,091,513, and U.S. Pat. No. 5,476,786. By way of example, effector proteins may include immunoglobulin constant region sequences. See, e.g., Hollenbaugh et al., 1995 J. Immunol. Methods 188:1-7. Other examples of effector proteins are enzymes. As a non-limiting example, such an enzyme may provide a biological activity for therapeutic purposes (see, e.g., Simmers et al., Bioconjug. Chem. 8:510-19 (1997)), or may provide a detectable activity, such as horseradish peroxidase-catalyzed conversion of any of a number of well-known substrates into a detectable product, for diagnostic uses.

The scFv may, in certain embodiments, be fused to peptide or polypeptide domains that permit detection of specific binding between the fusion protein and antigen (e.g., A2E). For example, the fusion polypeptide domain may be an affinity tag polypeptide. Binding of the scFv fusion protein to a binding partner (e.g., A2E or a derivative thereof described herein) may therefore be detected using an affinity polypeptide or peptide tag, such as an avidin, streptavidin or a His (e.g., polyhistidine) tag, by any of a variety of techniques with which those skilled in the art will be familiar. Detection techniques may also include, for example, binding of an avidin or streptavidin fusion protein to biotin or to a biotin mimetic sequence (see, e.g., Luo et al., J. Biotechnol. 65:225 (1998) and references cited therein), direct covalent modification of a fusion protein with a detectable moiety (e.g., a labeling moiety), non-covalent binding of the fusion protein to a specific labeled reporter molecule, enzymatic modification of a detectable substrate by a fusion protein that includes a portion having enzyme activity, or immobilization (covalent or non-covalent) of the fusion protein on a solid-phase support. An scFv fusion protein comprising an A2E-specific immunoglobulin-derived polypeptide may be fused to another polypeptide such as an effector peptide having desirable affinity properties (see, e.g, U.S. Pat. No. 5,100,788; WO 89/03422; U.S. Pat. No. 5,489,528; U.S. Pat. No. 5,672,691; WO 93/24631; U.S. Pat. No. 5,168,049; U.S. Pat. No. 5,272,254; EP 511,747). As provided herein, scFv polypeptide sequences may be fused to fusion polypeptide sequences, including effector protein sequences, that may include full-length fusion polypeptides and that may alternatively contain variants or fragments thereof.

Antibodies may also be identified and isolated from human immunoglobulin phage libraries, from mouse immunoglobulin phage libraries, from rabbit immunoglobulin phage libraries, and/or from chicken immunoglobulin phage libraries (see, e.g., Winter et al., 1994 Annu. Rev. Immunol. 12:433-55; Burton et al., Adv. Immunol. 57:191-280 (1994); U.S. Pat. No. 5,223,409; Huse et al., Science 246:1275-81 (1989); Schlebusch et al., Hybridoma 16:47-52 (1997) and references cited therein; Rader et al., J. Biol. Chem. 275:13668-76 (2000); Popkov et al., J. Mol. Biol. 325:325-35 (2003); Andris-Widhopf et al., J. Immunol. Methods 242:159-31 (2000)). Antibodies isolated from non-human species or non-human immunoglobulin libraries may be genetically engineered according to methods described herein and known in the art to “humanize” the antibody or fragment thereof. Immunoglobulin variable region gene combinatorial libraries may be created in phage vectors that can be screened to select Ig fragments (Fab, Fv, scFv, or multimers thereof) that bind specifically to A2E (see, e.g., U.S. Pat. No. 5,223,409; Huse et al., Science 246:1275-81 (1989); Sastry et al., Proc. Natl. Acad. Sci. USA 86:5728-32 (1989); Alting-Mees et al., Strategies in Molecular Biology 3:1-9 (1990); Kang et al., Proc. Natl. Acad. Sci. USA 88:4363-66 (1991); Hoogenboom et al., J. Molec. Biol. 227:381-388 (1992); Schlebusch et al., Hybridoma 16:47-52 (1997) and references cited therein; U.S. Pat. No. 6,703,015). For example, a library containing a plurality of polynucleotide sequences encoding Ig variable region fragments may be inserted into the genome of a filamentous bacteriophage, such as M13 or a variant thereof, in frame with the sequence encoding a phage coat protein such as gene III or gene VIII. A fusion protein may be a fusion of the coat protein with the light chain variable region domain and/or with the heavy chain variable region domain. According to certain embodiments, immunoglobulin Fab fragments may also be displayed on a phage particle (see, e.g., U.S. Pat. No. 5,698,426).

Heavy and light chain immunoglobulin cDNA expression libraries may also be prepared in lambda phage, for example, using λImmunoZap™(H) and λImmunoZap™(L) vectors (Stratagene, La Jolla, Calif.). Briefly, mRNA is isolated from a B cell population and used to create heavy and light chain immunoglobulin cDNA expression libraries in the λImmunoZap(H) and λImmunoZap(L) vectors. These vectors may be screened individually or co-expressed to form Fab fragments or antibodies (see Huse et al., supra; see also Sastry et al., supra). Positive plaques may subsequently be converted to a non-lytic plasmid that allows high level expression of monoclonal antibody fragments from E. coli.

Phage that display an Ig fragment (e.g., an Ig V-region or Fab) that binds to A2E may be selected by mixing the phage library with A2E or a derivative, or an A2E immunoconjugate as described herein, or by contacting the phage library with such A2E molecules immobilized on a solid matrix under conditions and for a time sufficient to allow binding. Unbound phage are removed by a wash, which typically may be a buffer containing salt (e.g., NaCl) at a low concentration, preferably with less than 100 mM NaCl, more preferably with less than 50 mM NaCl, most preferably with less than 10 mM NaCl, or, alternatively, a buffer containing no salt. Specifically bound phage (i.e., phage that contain an A2E specific Ig fragment) are then eluted with an NaCl-containing buffer, for example, by increasing the salt concentration in a step-wise manner. Typically, phage that bind A2E with higher affinity will require higher salt concentrations to be released. Eluted phage may be propagated in an appropriate bacterial host, and generally, successive rounds of A2E binding and elution can be repeated to increase the yield of phage expressing A2E-specific immunoglobulin.

Phage display techniques may also be used to select Ig fragments or single chain antibodies that bind to A2E. For examples of suitable vectors having multicloning sites into which candidate nucleic acid molecules (e.g., DNA) encoding such antibody fragments or related peptides may be inserted, see, e.g., McLafferty et al., Gene 128:29-36, (1993); Scott et al., Science 249:386-390 (1990); Smith et al., Meth. Enzymol. 217:228-257 (1993); Fisch et al., Proc. Natl. Acad. Sci. USA 93:7761-66 (1996)). The inserted DNA molecules may comprise randomly generated sequences, or may encode variants of a known peptide or polypeptide domain that specifically binds to A2E, as provided herein. Generally, the nucleic acid insert encodes a peptide of up to 60 amino acids, or may encode a peptide of 3 to 35 amino acids, or may encode a peptide of 6 to 20 amino acids. The peptide encoded by the inserted sequence is displayed on the surface of the bacteriophage. Phage expressing a binding domain for A2E may be selected on the basis of specific binding to an immobilized A2E or a derivative thereof or an A2E immunoconjugate as described herein. Well-known recombinant genetic techniques may be used to construct fusion proteins containing the fragment. For example, a polypeptide may be generated that comprises a tandem array of two or more similar or dissimilar affinity selected A2E binding peptide domains, in order to maximize binding affinity for A2E of the resulting product.

In certain other embodiments, A2E-specific antibodies are multimeric antibody fragments. Useful methodologies are described generally, for example in Hayden et al., Curr Opin. Immunol. 9:201-12 (1997); Coloma et al., Nat. Biotechnol. 15:159-63 (1997). For example, multimeric antibody fragments may be created by phage techniques to form miniantibodies (U.S. Pat. No. 5,910,573) or diabodies (Holliger et al., Cancer Immunol. Immunother. 45:128-130 (1997)). Multimeric fragments may be generated that are multimers of an A2E-specific Fv, or that are bispecific antibodies comprising an A2E-specific Fv noncovalently associated with a second Fv having a different antigen specificity (see, e.g., Koelemij et al., J. Immunother. 22:514-24 (1999)).

Introducing amino acid mutations into A2E-binding immunoglobulin molecules may be useful to increase the specificity or affinity for A2E, or to alter an effector function. Immunoglobulins with higher affinity for A2E may be generated by site-directed mutagenesis of particular residues. Computer assisted three-dimensional molecular modeling may be employed to identify the amino acid residues to be changed in order to improve affinity for A2E (see, e.g., Mountain et al., Biotechnol. Genet. Eng. Rev. 10:1-142 (1992)). Alternatively, combinatorial libraries of CDRs may be generated in M13 phage and screened for immunoglobulin fragments with improved affinity (see, e.g., Glaser et al., J. Immunol. 149:3903-3913 (1992); Barbas et al., Proc. Natl. Acad. Sci. USA 91:3809-13 (1994); U.S. Pat. No. 5,792,456).

In certain embodiments, the antibody may be genetically engineered to have an altered effector function. For example, the antibody may have an altered capability to mediate complement dependent cytotoxicity (CDC) or antibody dependent cellular cytotoxicity (ADCC). Effector functions may be altered by site-directed mutagenesis (see, e.g., Duncan et al., Nature 332:563-64 (1988); Morgan et al., Immunology 86:319-24 (1995); Eghtedarzedeh-Kondri et al., Biotechniques 23:830-34 (1997)). For example, mutation of the glycosylation site on the Fc portion of the immunoglobulin may alter the capability of the immunoglobulin to fix complement (see, e.g., Wright et al., Trends Biotechnol. 15:26-32 (1997)). Other mutations in the constant region domains may alter the ability of the immunoglobulin to fix complement, or to effect ADCC (see, e.g., Duncan et al., Nature 332:563-64(1988); Morgan et al., Immunology 86:319-24 (1995); Sensel et al., Mol. Immunol. 34:1019-29 (1997)). Alternatively, single chain polypeptides may be constructed recombinantly that comprise an A2E binding fragment, an immunoglobulin hinge region polypeptide, an immunoglobulin CH2 region polypeptide, and an immunoglobulin CH3 region polypeptide (see, e.g., U.S. Patent Publication Nos. 2003/0118592; 2003/0133939).

The nucleic acid molecules encoding an antibody or fragment thereof that specifically binds A2E, as described herein, may be propagated and expressed according to any of a variety of well-known procedures for nucleic acid excision, ligation, transformation, and transfection. Thus, in certain embodiments expression of an antibody fragment may be preferred in a prokaryotic host cell, such as Escherichia coli (see, e.g., Pluckthun et al., Methods Enzymol. 178:497-515 (1989)).

In certain other embodiments, expression of the antibody or an antigen-binding fragment thereof may be preferred in a eukaryotic host cell, including yeast (e.g., Saccharomyces cerevisiae, Schizosaccharomyces pombe, and Pichia pastoris); animal cells (including mammalian cells); or plant cells. Examples of suitable animal cells include, but are not limited to, myeloma, COS, CHO, or hybridoma cells. Examples of plant cells include tobacco, corn, soybean, and rice cells. By methods known to those having ordinary skill in the art and based on the present disclosure, a nucleic acid vector may be designed for expressing foreign sequences in a particular host system, and then polynucleotide sequences encoding the A2E binding antibody (or fragment thereof) may be inserted. The regulatory elements will vary according to the particular host.

Combinatorial phage libraries may also be used for humanization of non-human variable regions. See, e.g., Rosok et al., J. Biol. Chem. 271:22611-18 (1996); Rader et al., Proc. Natl. Acad. Sci. USA 95:8910-15 (1998)). The DNA sequence of the inserted immunoglobulin gene in the phage so selected may be determined by standard techniques (see, Sambrook et al., Molecular Cloning: A Laboratory Manual, Cold Spring Harbor Press (2001)). The affinity selected Ig-encoding sequence may then be cloned into another suitable vector for expression of the Ig fragment or, optionally, may be cloned into a vector containing Ig constant regions, for expression of whole immunoglobulin chains.

Similarly, portions or fragments, such as Fab and Fv fragments, of A2E specific antibodies may be constructed using conventional enzymatic digestion or recombinant DNA techniques to incorporate the variable regions of a gene that encodes a antibody specific for A2E. Within one embodiment, in a hybridoma the variable regions of a gene expressing a monoclonal antibody of interest are amplified using nucleotide primers. These primers may be synthesized by one of ordinary skill in the art, or may be purchased from commercially available sources (see, e.g., Stratagene (La Jolla, Calif.), which sells primers for mouse and human variable regions. The primers may be used to amplify heavy or light chain variable regions, which may then be inserted into vectors such as ImmunoZAP™ H or ImmunoZAP™ L (Stratagene), respectively. These vectors may then be introduced into E. coli, yeast, or mammalian-based systems for expression. Large amounts of a single-chain protein containing a fusion of the V_{H} and V_{L} domains may be produced using these methods (see Bird et al., Science 242:423-426 (1988)). In addition, such techniques may be used to humanize a non-human antibody V region without altering the binding specificity of the antibody.

One or more replicable expression vectors containing DNA encoding a variable and/or constant region may be prepared and used to transform an appropriate cell line, for example, a non-producing myeloma cell line, such as a mouse NSO line or a bacteria, such as E. coli, in which production of the antibody will occur. In order to obtain efficient transcription and translation, the DNA sequence in each vector should include appropriate regulatory sequences, particularly a promoter and leader sequence operatively linked to the variable domain sequence. Particular methods for producing antibodies in this way are generally well known and routinely used. For example, molecular biology procedures are described by Sambrook et al. (Molecular Cloning, A Laboratory Manual, 2nd ed., Cold Spring Harbor Laboratory, New York, 1989; see also Sambrook et al., 3rd ed., Cold Spring Harbor Laboratory, New York, (2001)). DNA sequencing can be performed as described in Sanger et al. (Proc. Natl. Acad. Sci. USA 74:5463 (1977)) and the Amersham International plc sequencing handbook and including improvements thereto.

Site directed mutagenesis of an immunoglobulin variable (V region), framework region, and/or constant region may be performed according to any one of numerous methods described herein and practiced in the art (Kramer et al., Nucleic Acids Res. 12:9441 (1984); Kunkel Proc. Natl. Acad. Sci. USA 82:488-92 (1985); Kunkel et al., Methods Enzymol. 154:367-82 (1987)). Random mutagenesis methods to identify residues that are either important to binding to A2E or that do not alter binding of the antigen to the antibody when altered can also be performed according to procedures that are routinely practiced by a person skilled in the art (e.g., alanine scanning mutagenesis; error prone polymerase chain reaction mutagenesis; and oligonucleotide-directed mutagenesis (see, e.g., Sambrook et al. Molecular Cloning: A Laboratory Manual, 3rd ed., Cold Spring Harbor Laboratory Press, NY (2001))). Additionally, numerous publications describe techniques suitable for the preparation of antibodies by manipulation of DNA, creation of expression vectors, and transformation of appropriate cells (Mountain et al., in Biotechnology and Genetic Engineering Reviews (ed. Tombs, M P, 10, Chapter 1, Intercept, Andover, UK (1992)); International Patent Publication No. WO 91/09967).

In certain embodiments, an antibody that specifically binds to A2E may be used to detect A2E in a cell or in a biological sample. For such a purpose an A2E-binding immunoglobulin (or fragment thereof) as described herein may contain a detectable moiety or label such as an enzyme, cytotoxic agent, or other reporter molecule, including a dye, radionuclide, luminescent group, fluorescent group, or biotin, or the like. The A2E-specific immunoglobulin or fragment thereof may be radiolabeled for diagnostic or therapeutic applications. Techniques for radiolabeling of antibodies are known in the art (see, e.g., Adams, In Vivo 12:11-21 (1998); Hiltunen, Acta Oncol. 32:831-9 (1993)). The effector or reporter molecules may be attached to the antibody through any available amino acid side-chain, terminal amino acid, or carbohydrate functional group located in the antibody, provided that the attachment or attachment process does not adversely affect the binding properties such that the usefulness of the molecule is abrogated. Particular functional groups include, for example, any free amino, imino, thiol, hydroxyl, carboxyl, or aldehyde group. Attachment of the antibody or antigen-binding fragment thereof and the effector and/or reporter molecule(s) may be achieved via such groups and an appropriate functional group in the effector or reporter molecule. The linkage may be direct or indirect through spacing or bridging groups (see, e.g., WO 93/06231, WO 92/22583, WO 90/091195, and WO 89/01476; see also, e.g., commercial vendors such as Pierce Biotechnology, Rockford, Ill.).

As provided herein and according to methodologies well known in the art, polyclonal and monoclonal antibodies may be used for the affinity isolation of A2E and derivatives thereof and A2E immunoconjugates (see, e.g., Hermanson et al., Immobilized Affinity Ligand Techniques, Academic Press, Inc. New York, (1992)). Briefly, an antibody (or antigen-binding fragment thereof) may be immobilized on a solid support material, which is then contacted with a sample that contains A2E. The sample interacts with the immobilized antibody under conditions and for a time that are sufficient to permit binding of A2E to the immobilized antibody; non-binding components (that is, those components unrelated to A2E) of the sample are removed; and then A2E is released from the immobilized antibody using an appropriate solution for eluting A2E.

In certain embodiments, anti-idiotype antibodies that recognize an antibody (or antigen-binding fragment thereof) that specifically binds to A2E are provided and methods for using these anti-idiotype anatibodies. Anti-idiotype antibodies may be generated as polyclonal antibodies or as monoclonal antibodies by the methods described herein, using an anti-A2E antibody (or antigen-binding fragment thereof) as immunogen. Anti-idiotype antibodies or fragments thereof may also be generated by any of the recombinant genetic engineering methods described above or by phage display selection. An anti-idiotype antibody may react with the antigen-binding site of the anti-A2E antibody such that binding of the antibody to A2E is competitively inhibited. Alternatively, an anti-idiotype antibody as provided herein may not competitively inhibit binding of an anti-A2E antibody to A2E. Anti-idiotype antibodies are useful for immunoassays to determine the presence of anti-A2E antibodies in a biological sample. For example, an immunoassay such as an ELISA, which are practiced by persons skilled in the art, may be used to determine the presence of an immune response induced by administering (i.e., immunizing) a host with an A2E immunoconjugate as described herein.

In certain embodiments of the invention, an antibody specific for A2E may be an antibody or antigen-binding fragment thereof that is expressed as an intracellular protein. Such intracellular antibodies are also referred to as intrabodies and may comprise an Fab fragment, a Fv fragment, a scFv molecule, an scFv-Fc fusion antibody, or a bispecific antibody, all of which may be made as described herein and according to methods practiced in the art (see, e.g., Lobato et al., Curr. Mol. Med. 4:519-28 (2004); Strube et al., Methods 34:179-83 (2004); Lecerf et al., Proc. Natl. Acad. Sci. USA 98:4764-49 (2001); (Weisbart et al., Int. J. Oncol. 25:1113-18 (2004)). The framework regions flanking the CDR regions can be modified to improve expression levels, stability, and/or solubility of an intrabody in an intracellular reducing environment (see, e.g., Auf der Maur et al., Methods 34:215-24 (2004); Strube et al., supra; Worn et al., J. Biol. Chem. 275:2795-803 (2000)). An intrabody may be directed to a particular cellular location or organelle, for example by constructing a vector that comprises a polynucleotide sequence encoding the variable regions of an intrabody that may be operatively fused to a polynucleotide sequence that encodes a particular target antigen within the cell (see, e.g., Graus-Porta et al., Mol. Cell Biol. 15:1182-91 (1995); Lener et al., Eur. J. Biochem. 267:1196-205 (2000); Popkov et al., Cancer Res. 65:972-81 (2005)). Various types of intrabodies have been investigated as therapeutic agents for treating cancer (see, e.g., Weisbart et al., supra; Popkov et al., supra; Krauss et al., Breast Dis. 11:113-24 (1999)) and for treating neurodegenerative diseases such as Parkinson's disease (Zhou et al., Mol. Ther. 10:1023-31 (2004)) and Huntington's disease (Murphy et al., Brain Res. Mol. Brain Res. 121:141-45 (2004); Colby et al., J. Mol. Biol. 342:901-12 (2004); Colby et al., Proc. Natl. Acad. Sci. USA 101:17616-21 (2004), Erratum in Proc. Natl. Acad. Sci. USA 102:955 (2005)). An intrabody may be introduced into a cell by a variety of techniques available to the skilled artisan including via a gene therapy vector, a lipid mixture (e.g., Provectin™ manufactured by Imgenex Corporation, San Diego, Calif.), photochemical internalization methods, or other methods known in the art.

In Vivo and In Vitro Systems for Determining Effect of Anti-A2E Antibodies

In one embodiment, methods are provided for inhibiting degeneration of a retinal cell or enhancing or prolonging retinal cell survival, including retinal neuronal cell survival, using antibodies that bind specifically to A2E. In another embodiment, methods are provided for enhancing or prolonging retinal cell survival or inhibiting degeneration of a retinal cell, by inducing an immune response in a host by immunizing (administering to the host) an A2E immunoconjugate as described herein that acts or functions as a vaccine. These compounds are useful for inhibiting degeneration and/or enhancing retinal cell survival, including photoreceptor cell survival, which can result in slowing or halting the progression of macular degeneration, or retinal blood vessel occlusion, or other eye diseases that are related to neurodegenerative diseases such as Alzheimer's disease and Parkinson's disease. A retinal cell includes a retinal neuronal cell or other mature retinal cell, such as a retinal pigmented epithelium (RPE) cell or a Müller glial cell. Retinal neuronal cells include photoreceptor cells, horizontal cells, amacrine cells, bipolar cells, and ganglion cells.

The effect of an A2E specific antibody on retinal cell survival may be determined by using cell culture models, animal models, and other methods that are described herein and practiced by persons skilled in the art. By way of example, and not limitation, such methods and assays include those described in Oglivie et al., Exp. Neurol. 161:675-856 (2000); U.S. Pat. No. 6,406,840; WO 01/81551; WO 98/12303; U.S. Patent Application No. 2002/0009713; WO 00/40699; U.S. Pat. No. 6,117,675; U.S. Pat. No. 5,736,516; WO 99/29279; WO 01/83714; WO 01/42784; U.S. Pat. No. 6,183,735; U.S. Pat. No. 6,090,624; WO 01/09327; U.S. Pat. No. 5,641,750; and U.S. patent application Ser. No. 10/903,880.

The lack of a good animal model has proved to be a major obstacle for developing new drugs to treat retinal diseases and disorders. For example, macula exist in primates (including humans) but not in rodents. A recently developed animal model may be useful for evaluating treatments for macular degeneration has been described by Ambati et al. (Nat. Med. 9:1390-97 (2003); Epub 2003 Oct. 19). This animal model is one of only a very few exemplary animal models presently available for evaluating a compound or any molecule for use in treating (including preventing) progression or development of a neurodegenerative disease, especially an ophthalmic disease. Accordingly, cell culture methods, such as the method described herein, is particularly useful for determining the effect of on retinal neuronal cell survival.

Cell Culture System

An exemplary cell culture model is described herein that is useful for determining the capability of an anti-A2E antibody or an antigen-binding fragment thereof as described herein to enhance or prolong survival of retinal cells, including retinal neuronal cells, and mature retinal cells such as RPE and Müller glia, and which molecules are useful for treating macular degeneration, such as dry form macular degeneration (see also U.S. patent application Ser. No. 10/903,880).

The cell culture model comprises a long-term or extended culture of mature retinal cells, including retinal neuronal cells (e.g., photoreceptor cells, amacrine cells, ganglion cells, horizontal cells, and bipolar cells). The cell culture system and methods for producing the cell culture system provide extended culture of photoreceptor cells. The cell culture system described herein may also comprise retinal pigmented epithelial (RPE) cells and Müller glial cells.

The retinal cell culture system may also comprise a cell stressor. The application or the presence of the stressor affects the mature retinal cells, including the retinal neuronal cells, in vitro in a manner that is useful for studying disease pathology that is observed in a retinal disease or disorder. The cell culture model described herein provides an in vitro neuronal cell culture system that will be useful in the identification and biological testing of an anti-A2E antibody or antigen-binding fragment thereof that is suitable for treatment of neurological diseases or disorders in general, and for treatment of degenerative diseases of the eye and brain in particular. The ability to obtain primary cells from mature, fully-differentiated retinal cells, including retinal neurons for culture in vitro over an extended period of time in the presence of a stressor enables examination of cell-to-cell interactions, selection and analysis of neuroactive compounds and materials, use of a controlled cell culture system for in vivo CNS and ophthalmic tests, and analysis of the effects on single cells from a consistent retinal cell population.

The cell culture system described herein and the retinal cell stress model comprise cultured mature retinal cells, retinal neurons, and a retinal cell stressor, which are particularly useful for screening bioactive agents capable of inducing or stimulating regeneration of CNS tissue that has been damaged by disease. The cultured mature retinal neurons comprise all the major retinal neuronal cell types including photoreceptor, amacrine, ganglion cells, horizontal cells, and bipolar cells.

The in vitro cell culture system permits and promotes (or extends) the survival (or inhibits degeneration of the cells) in culture of mature retinal cells, including retinal neurons, for over 2 months and for as long as 6 months. In other cell culture systems, the ability to screen drug candidates using mature retinal cells has been limited to the life span of the retinal cells (between one and two weeks), particularly the retinal neurons, in primary culture. See also, e.g., Luo et al., Invest. Ophthalmol. Vis. Sci. 42:1096-1106 (2001); Gaudin et al., Invest. Ophthalmol. Vis. Sci. 37:2258-68 (1996). Delays in enucleation and delays in tissue dissociation have a severe deleterious effect on recovery and survival of neurons (see, for example, Gaudin et al., supra). Neurons begin to deteriorate immediately after being dissociated from the animal body, and the resulting deterioration precludes adequate and reliable compound screening to identify agents that may be used for treating retinal diseases. Also, without the ability to maintain a long-term retinal cell culture, performing various analyses related to either projection neurons or photoreceptor cells is difficult. Photoreceptors are the primary cell type affected in macular degeneration, a leading cause of blindness. Ganglion cells, projection neurons in the retina, are affected in glaucoma patients, also a leading cause of blindness.

This cell culture system comprises the culture of retinal cells including retinal neurons in vitro for extended periods of time, thus providing viable, fully mature retinal cells and neurons for a period greater than 2 months. Also provided herein is a method for producing the cell culture system comprising isolating mature retinal cells from a biological source and culturing the mature retinal cells under conditions that maintain viability of the mature retinal cells. Viability of the retinal cells in the cell culture system means that all or a portion of the cells that are isolated and plated for tissue culture as described herein metabolize and exhibit structure and functions of a healthy, thriving cell that is characteristic for the particular cell type. Viability of one or more of the mature retinal cell types is maintained for an extended period of time, for example, at least 4 weeks, 2 months (8 weeks), or at least 4-6 months, for at least 10%, 25%, 40%, 50%, 60%, 70%, 80%, or 90% of the mature retinal cells that are isolated (harvested) from retinal tissue and plated for tissue culture. Viability of the retinal cells may be determined according to methods described herein and known in the art. Retinal neuronal cells, similar to neuronal cells in general, are not actively dividing cells in vivo and thus cell division of retinal neuronal cells would not necessarily be indicative of viability. An advantage of the cell culture system is the ability to culture amacrine cells, photoreceptors, and associated ganglion projection neurons for extended periods of time, thereby providing an opportunity to determine the effectiveness of an anti-A2E antibody or antigen-binding fragment thereof described herein for treatment of retinal disease.

Such a chronic disease model is of particularly importance because most neurodegenerative diseases are chronic. In addition, through use of this in vitro cell culture system, the earliest events in long-term disease development processes may be identified because an extended period of time is available for cellular analysis. The long-term mature retinal culture system described herein also is useful for experiments that are relatively short term in duration (e.g., 3-14 days) because the baseline for survival and viability is more stable than in short-term culture models heretofore developed in which the cells are progressively dying.

The cell culture system described herein provides a mature retinal cell culture that is a mixture of mature retinal neuronal cells and non-neuronal retinal cells. The cell culture system may comprise all the major retinal neuronal cell types (photoreceptors, bipolar cells, horizontal cells, amacrine cells, and ganglion cells), and also includes other mature retinal cells such as RPE and Müller glial cells. By incorporating these different types of cells into the in vitro culture system, the system essentially resembles an “artificial organ” that is more akin to the natural in vivo state of the retina.

The mature retinal cells and retinal neurons may be cultured in vitro for extended periods of time, longer than 2 days or 5 days, longer than 2 weeks, 3 weeks, or 4 weeks, and longer than 2 months (8 weeks), 3 months (12 weeks), and 4 months (16 weeks), and longer than 6 months, thus providing a long-term culture. At least 20-40%, at least 50%, at least 60%, at least 70%, at least 80%, or at least 90% of one or more of the mature retinal cell types remain viable in this long-term cell culture system. The biological source of the retinal cells or retinal tissue may be mammalian (e.g., human, non-human primate, ungulate, rodent, canine, porcine, bovine, or other mammalian source), avian, or from other genera. Retinal cells including retinal neurons from post-natal non-human primates, post-natal pigs, or post-natal chickens may be used, but any adult or post-natal retinal tissue may be suitable for use in this retinal cell culture system. The types of retinal neuronal cells that may be cultured in vitro by this method include ganglion cells, photoreceptors, bipolar cells, horizontal cells, and amacrine cells. Non-neuronal retinal cells that are cultured with the retinal neurons are cells that are derived from the original retinal tissue, and include, for example, RPE cells and Müller glial cells.

The cell culture system described herein provides for robust long-term survival of retinal cells without inclusion of cells derived from or isolated or purified from non-retinal tissue. The cell culture system comprises cells isolated solely from the retina of the eye and thus is substantially free of types of cells from other parts or regions of the eye that are separate from the retina, such as ciliary bodies and vitreous. A retinal cell culture that is substantially free of non-retinal cells contains retinal cells that comprise preferably at least 80-85% of the cell types in culture, preferably 90%-95%, or preferably 96%-100% of the cell types. Retinal cells in the cell culture system are viable and survive in the cell culture system without added purified (or isolated) glial cells or stem cells from a non-retinal source, or other non-retinal cells. As described herein the retinal cell culture system is prepared from isolated retinal tissue only, thereby rendering the cell culture system substantially free of non-retinal cells.

Persons skilled in the cell culture art appreciate that successfully obtaining a long-term or extended culture of cells derived directly from a tissue source (i.e., a primary cell culture) and maintaining viability of the cells (e.g., retinal cells) in culture depends on several factors. Similar to establishing a long-term culture of any tissue-derived cell population (even including tumor tissue for propagation of immortalized cancer cells), the length of time that passes between harvesting of a retinal tissue and plating of the cells can particularly affect successful establishment of a long term culture. Neurons begin to deteriorate immediately after being dissociated from neural tissue. Delays in enucleation and delays in tissue dissociation have a severe deleterious effect on recovery and survival of neurons (see, for example, Gaudin et al, supra).

Accordingly, methods for producing an extended retinal cell culture may benefit from minimizing the time periods between harvesting the tissue (which also includes minimizing the time between the death of the source animal and when the tissue is harvested) and dissecting the tissue, and the time between initiation and completion of the dissection and dissociation procedures and plating of the cells. For example, to prepare the retinal cell culture, the eyes that are dissected are preferably obtained and dissected within 12 hours of harvesting the organ. In addition, the dissection methods are performed more quickly than previously described methods for culturing retinal cells. The efficiency of this method is improved over methods for production of other retinal cell culture systems that combine retinal cells with other cell types from the eye or other regions of the CNS, by eliminating those additional cell preparation steps. Other factors that can affect successful culturing of tissue-derived cells include the temperature at which the tissues are maintained during and after transport, the health and age of the tissue donor, the skill of the animal handler, surgeon, and/or cell culturist, and similar factors appreciated by those skilled in the art.

Dissection of the eye may be performed according to standard procedures known in the art and described herein. By way of example, eyes obtained from a donor animal are enucleated, and muscle and other tissue are cleaned away from the eye orbit. In one cell culture system, the peripheral retina is dissected from other portions or regions of the eye. The eyes are cut in half along their equator, and the neural retina is dissected from the anterior part of the eye. The retina, ciliary body, and vitreous are dissected away from the anterior half portion of the eye in a single piece, followed by gentle detachment of the opaque retina from the clear vitreous. In another cell culture system, the posterior portion of the retina containing the area centralis is isolated from other regions of the eye by dissection. The posterior portion of the retina contains the fovea (and the macula in primates), with a higher concentration of cone photoreceptors, whereas the anterior portion of the retina has a higher concentration of rod photoreceptors. Pigmented epithelial cells may or may not be totally separated from the dissected retina.

Retinal cells may be isolated from retinal tissue by mechanical means, such as dissection and teasing (trituration). Tissues of the eye may also be treated with one or more enzymes including but not limited to papain, hyaluronidase, collagenase, trypsin, and/or a deoxyribonuclease, to dissociate the cells and remove undesired cellular components. The cell culture system may be prepared by a combination of mechanical methods and enzymatic digestion.

The cell culture systems and methods described herein may employ use of any plastic or glass surface (including, for instance, coverslips), preferably surfaces that are manufactured for cell culture use for providing a surface to which the retinal cells can adhere. The surface may also be coated with an attachment-enhancing substance or a combination of such substances, such as poly-lysine, Matrigel, laminin, polyornithine, gelatin, and/or fibronectin, or the like. Retinal cells prepared from an eye as described herein may be plated onto one surface, such as a glass coverslip, which is then placed in a tissue culture container and immersed in tissue culture media. The tissue culture container may be, for example, a multi-well plate such as a 24-well tissue culture plate. Alternatively, one or more surfaces onto which the retinal cells are plated (and to which the cells will adhere) may be placed in one or more tissue culture flasks, which are familiar to persons in the art. Alternatively, the retinal cells may be applied to and maintained in standard tissue culture multi-well dishes and/or tissue culture flasks. Feeder cell layers, such as glial feeder layers, epithelial cell layers, or embryonic fibroblast feeder layers, may also find use within the methods and systems provided herein.

For maintaining viability of the retinal cells in the cell culture system, the system also comprises components and conditions known in the art for proper maintenance of cells in culture, including media (with or without antibiotics) that contains buffers and nutrients (e.g., glucose, amino acids (e.g., glutamine), salts, minerals (e.g., selenium)) and also may contain other additives or supplements (e.g., fetal bovine serum or an alternative formulation that does not require a serum supplement; transferrin; insulin; putrescine; progesterone) that are required or are beneficial for in vitro culture of cells and that are well known to a person skilled in the art (see, for example, Gibco media, Invitrogen Life Technologies, Carlsbad, Calif.). Similar to standard cell culture methods and practices, the retinal cell cultures described herein are maintained in tissue culture incubators designed for such use so that the levels of carbon dioxide, humidity, and temperature can be controlled. The cell culture system may also comprise addition of exogenous (i.e., not produced by the cultured cells themselves) cell growth factors or neurotrophic factors, such as including but not limited to ciliary neurotrophic factor (CNTF), brain-derived neurotrophic factor (BDNF), fibroblast growth factor-2 (FGF2), and glial cell line-derived neurotrophic factor (GDNF), which may be provided, for example, in the media or in the substrate or surface coating.

Retinal Neuronal Cell Culture Stress Model

The in vitro retinal cell culture systems described herein may serve as physiological retinal models that can be used to characterize the physiology of the retina. This physiological retinal model may also be used as a broader general neurobiology model. A cell stressor may be included in the model cell culture system. A cell stressor, which as described herein is a retinal cell stressor, adversely affects the viability or reduces the viability of one or more of the different retinal cell types, including types of retinal neuronal cells, in the cell culture system. A person skilled in the art would readily appreciate and understand that as described herein a retinal cell that exhibits reduced viability means that the length of time that a retinal cell survives in the cell culture system is reduced or decreased (decreased lifespan) and/or that the retinal cell exhibits a decrease, inhibition, or adverse effect of a biological or biochemical function (e.g., decreased or abnormal metabolism; initiation of apoptosis; etc.) compared with a retinal cell cultured in an appropriate control cell system (e.g., the cell culture system described herein in the absence of the cell stressor). Reduced viability of a retinal cell may be indicated by cell death; an alteration or change in cell structure or morphology; induction and/or progression of apoptosis; initiation, enhancement, and/or acceleration of retinal neuronal cell neurodegeneration (or neuronal cell injury).

Methods and techniques for determining cell viability are described in detail herein and are those with which skilled artisans are familiar. These methods and techniques for determining cell viability may be used for monitoring the health and status of retinal cells in the cell culture system and for determining the capability of the anti-A2E antibodies and antigen-binding fragments thereof described herein to alter (preferably increase, prolong, enhance, improve) retinal cell viability or retinal cell survival and inhibit degeneration of a retinal cell.

The addition of a cell stressor to the cell culture system is useful for determining the capability of an anti-A2E antibody to abrogate, inhibit, eliminate, or lessen the effect of the stressor. The retinal neuronal cell culture system may include a cell stressor that is chemical (e.g., A2E, cigarette smoke concentrate); biological (for example, toxin exposure; beta-amyloid; lipopolysaccharides); or non-chemical, such as a physical stressor, environmental stressor, or a mechanical force (e.g., increased pressure or light exposure).

The retinal cell stressor model system may also include a cell stressor such as, but not limited to, a stressor that may be a risk factor in a disease or disorder or that may contribute to the development or progression of a disease or disorder, including but not limited to, light of varying wavelengths and intensities; cigarette smoke condensate exposure; glucose oxygen deprivation; oxidative stress (e.g., stress related to the presence of or exposure to hydrogen peroxide, nitroprusside, Zn++, or Fe++); increased pressure (e.g., atmospheric pressure or hydrostatic pressure), glutamate or glutamate agonist (e.g., N-methyl-D-aspartate (NMDA); alpha-amino-3-hydroxy-5-methylisoxazole-4-proprionate (AMPA); kainic acid; quisqualic acid; ibotenic acid; quinolinic acid; aspartate; trans-1-aminocyclopentyl-1,3-dicarboxylate (ACPD)); amino acids (e.g., aspartate, L-cysteine; beta-N-methylamine-L-alanine); heavy metals (such as lead); various toxins (for example, mitochondrial toxins (e.g., malonate, 3-nitroproprionic acid; rotenone, cyanide); MPTP (1-methyl-4-phenyl-1,2,3,6,-tetrahydropyridine), which metabolizes to its active, toxic metabolite MPP+ (1-methyl-4-phenylpryidine)); 6-hydroxydopamine; alpha-synuclein; protein kinase C activators (e.g., phorbol myristate acetate); biogenic amino stimulants (for example, methamphetamine, MDMA (3-4 methylenedioxymethamphetamine)); or a combination of one or more stressors. Useful retinal cell stressors include those that mimic a neurodegenerative disease that affects any one or more of the mature retinal cells described herein. A chronic disease model is of particular importance because most neurodegenerative diseases are chronic. Through use of this in vitro cell culture system, the earliest events in long-term disease development processes may be identified because an extended period of time is available for cellular analysis.

A retinal cell stressor may alter (i.e., increase or decrease in a statistically significant manner) viability of retinal cells such as by altering survival of retinal cells, including retinal neuronal cells, or by altering neutodegeneration of retinal neuronal cells. Preferably, a retinal cell stressor adversely affects a retinal neuronal cell such that survival of a retinal neuronal cell is decreased or adversely affected (i.e., the length of time during which the cells are viable is decreased in the presence of the stressor) or neurodegeneration (or neuron cell injury) of the cell is increased or enhanced. The stressor may affect only a single retinal cell type in the retinal cell culture or the stressor may affect two, three, four, or more of the different cell types. For example, a stressor may alter viability and survival of photoreceptor cells but not affect all the other major cell types (e.g., ganglion cells, amacrine cells, horizontal cells, bipolar cells, RPE, and Müller glia). Stressors may shorten the survival time of a retinal cell (in vivo or in vitro), increase the rapidity or extent of neurodegeneration of a retinal cell, or in some other manner adversely affect the viability, morphology, maturity, or lifespan of the retinal cell.

The effect of a cell stressor on the viability of retinal cells in the cell culture system may be determined for one or more of the different retinal cell types. Determination of cell viability may include evaluating structure and/or a function of a retinal cell continually at intervals over a length of time or at a particular time point after the retinal cell culture is prepared. Viability or long term survival of one or more different retinal cell types or one or more different retinal neuronal cell types may be examined according to one or more biochemical or biological parameters that are indicative of reduced viability, such as apoptosis or a decrease in a metabolic function, prior to observation of a morphological or structural alteration.

A chemical, biological, or physical cell stressor may reduce viability of one or more of the retinal cell types present in the cell culture system when the stressor is added to the cell culture under conditions described herein for maintaining the long-term cell culture. Alternatively, one or more culture conditions may be adjusted so that the effect of the stressor on the retinal cells can be more readily observed. For example, the concentration or percent of fetal bovine serum may be reduced or eliminated from the cell culture when cells are exposed to a particular cell stressor. When a serum-free media is desired for a particular purpose, cells may be gradually weaned (i.e., the concentration of the serum is progressively and often systematically decreased) from an animal source of serum into a media that is free of serum or that contains a non-serum substitute. The decrease in serum concentration and the time period of culture at each decreased concentration of serum may be continually evaluated and adjusted to ensure that cell survival is maintained. When the retinal cell culture system described herein is exposed to a cell stressor, the serum concentration may be adjusted concomitantly with the application of the stressor (which may also be titrated (if chemical or biological) or adjusted (if a physical stressor)) to achieve conditions such that the stress model is useful for evaluating the effect of the stressor on a retinal cell type and/or for identifying an agent that inhibits, reduces, or abrogates the adverse effect(s) of a stressor on the retinal cell. Alternatively, retinal cells cultured in media containing serum at a particular concentration for maintenance of the cells may be abruptly exposed to media that does not contain any level of serum. In another embodiment, serum may be decreased in a retinal cell culture to less than 5%, 2%, 1%, 0.5%, less than 0.25%, less than 0.1%, or less than 0.05% in a single step.

The retinal cell culture may be exposed to a cell stressor for a period of time that is determined to reduce the viability of one or more retinal cell types in the retinal cell culture system. The length of time that the culture is exposed to a cell stressor may be 3 hours, 6 hours, 9 hours, 12, hours, 18 hours, 24 hours, or 2 days, 3 days, 4 days, 5 days, 6 days, or a week, at least two weeks, and at least one month, or longer, or for any period of time between the time periods enumerated. The cells may be exposed to a cell stressor immediately upon plating of the retinal cells after isolation from retinal tissue. Alternatively, the retinal cell culture may be exposed to a stressor after the culture is established, or any time thereafter (e.g., one day, two days, 3-5 days, 6-10 days, 2 weeks, 3 weeks, or 4 weeks). When two or more cell stressors are included in the retinal cell culture system, each stressor may be added to the cell culture system concurrently and for the same length of time or may be added separately at different time points for the same length of time or for differing lengths of time during the culturing of the retinal cell system.

Viability of the retinal cells in the cell culture system may be determined by any one or more of several methods and techniques described herein and practiced by skilled artisans (see also, e.g., methods and techniques described herein regarding determining viability in the presence of an anti-A2E antibody or antigen-binding fragment described herein. The effect of a stressor may be determined by comparing structure or morphology of a retinal cell, including a retinal neuronal cell, in the cell culture system in the presence of the stressor with structure or morphology of the same cell type of the cell culture system in the absence of the stressor, and therefrom identifying a stressor that is capable of altering neurodegeneration of the neuronal cell. The effect of the stressor on viability can also be evaluated by methods known in the art and described herein, for example by comparing survival of a neuronal cell of the cell culture system in the presence of the stressor with survival of a neuronal cell of the cell culture system in the absence of the stressor.

Survival of retinal cells may be determined according to methods described in detail herein and known in the art that identify and characterize retinal cells, for example, immunocytochemical methods. Antibodies that specifically bind to cell markers for a specific retinal or retinal neuronal cell type as well as antibodies that bind to cytoskeletal proteins common to more than one cell type are commercially available. Alternatively, such antibodies can be prepared according to standard methods and techniques known in the art (see, e.g., Kohler and Milstein, Eur. J. Immunol. 6:511-519 (1976) and improvements thereto; Harlow and Lane, Antibodies: A Laboratory Manual, Cold Spring Harbor Laboratory (1988); Antibody Engineering, Methods and Protocols, Lo, ed., (Human Press 2004); U.S. Pat. Nos. 5,693,762; 5,585,089; 4,816,567; 5,225,539; 5,530,101; U.S. Pat. No. 5,223,409; Schlebusch et al., Hybridoma 16:47 (1997); and references cited therein; see also Andris-Widhopf et al., J. Immunol. Methods 242:159-81 (2000)).

Photoreceptors may be identified using antibodies that specifically bind to photoreceptor-specific proteins such as opsins, peripherins, and the like. Photoreceptors in cell culture may also be identified as a morphologic subset of immunocytochemically labeled cells by using a pan-neuronal marker or may be identified morphologically in enhanced contrast images of live cultures. Outer segments can be detected morphologically as attachments to photoreceptors.

Retinal cells including photoreceptors can also be detected by functional analysis. For example, electrophysiology methods and techniques may be used for measuring the response of photoreceptors to light. Photoreceptors exhibit specific kinetics in a graded response to light. Calcium-sensitive dyes may also be used to detect graded responses to light within cultures containing active photoreceptors. For analyzing stress-inducing compounds or potential neurotherapeutics, retinal cell cultures can be processed for immunocytochemistry, and photoreceptors and/or other retinal cells can be counted manually or by computer software using photomicroscopy and imaging techniques. Other immunoassays known in the art (e.g., ELISA, immunoblotting, flow cytometry) may also be useful for identifying and characterizing the retinal cells and retinal neuronal cells of the cell culture model system described herein.

The retinal cell culture stress models may also be useful for identification of both direct and indirect pharmacologic agent effects by the bioactive agent of interest, such as an antibody, or antigen-binding fragment thereof, that specifically binds to A2E. For example, a bioactive agent added to the cell culture system in the presence of one or more retinal cell stressors may stimulate one cell type in a manner that enhances or decreases the survival of other cell types. Cell/cell interactions and cell/extracellular component interactions may be important in understanding mechanisms of disease and drug function. For example, one neuronal cell type may secrete trophic factors that affect growth or survival of another neuronal cell type (see, e.g., WO 99/29279).

Light Stressor

Light is believed to cause or contribute to retinal cell death, particularly photoreceptor cell death. Exposure to cumulative amounts of light is considered a risk factor for onset of macular degeneration. The results from animal studies have indicated that mice exposed to high intensity light develop similar pathophysiological effects as observed in humans with macular degeneration (see, e.g., Dithmar et al., Arch. Ophthalmol. 119:1643-49 (2001); Gottsch et al., Arch. Ophthalmol. 111:126-29 (1993)).

For culture of retinal cells exposed to a light stressor, the light may be emitted from at least one fluorescent light, incandescent light, or at least one light-emitting diode. The exposure may be intermittent or constant, and the duration of exposure may be varied. Alternatively, light stress may be applied as a light shock whereby cells at some point prior to or during cell culture may be protected from exposure to any light source and then exposed to a light stress.

The intensity of the light stress may be measured in lux, which is a measure of light output at a surface. The retinal cell culture described herein is preferably exposed to light (white or blue light) at any intensity or at any range of intensities from about 1 to 20,000 lux, at any intensity or any range of intensities between about 1000-15,000 lux, between about 1000-8000 lux, between about 250-8000 lux, 250-1000 lux, 250-2000 lux, 250-4000 lux, between about 4000-8000 lux, between about 1000-6000 lux, between about 1000-4000, between about 2000-6000, between about 2000-4000, between about 4000-6000 lux, or between about 1000-2000 lux. By way of example, cells are exposed to moderate intensity, for example, about 4000-6000 lux over a short period of time, for example, less than one week, between 18-96 hours, or between 18-48 hours. In another embodiment, the retinal cells are exposed to lower intensity of light (for example, between about 500-4000 lux, or between about 500-2000 lux, between about 250-1000, or between about 500-1000 lux) over a longer period of time (such as, longer than one week, at least two weeks, or at least one month). The latter set of conditions (lower intensity of light over a longer period of time) may provide a stress model for evaluating the effect of stress in chronic neurodegenerative retinal diseases and thus for determining the capability of an anti-A2E antibody, or antigen-binding fragment thereof, described herein to treat chronic neurodegenerative retinal diseases. In a particular embodiment, the light stressor is a blue light. As described herein A2E is phototoxic and initiates blue light-induced apoptosis in RPE cells (see, e.g., Sparrow et al., Invest. Ophthalmol. Vis. Sci. 43:1222-27 (2002)). Upon exposure to blue light, photooxidative products of A2E are formed (e.g., epoxides) that damage cellular macromolecules, including DNA (Sparrow et al., J. Biol. Chem. 278(20): 18207-13 (2003)).

The light stress may comprise ultraviolet or visible light at any wavelength varying from between 100 to 700 nm. The light stress may be visible light and include light at any wavelength from approximately 400 nm (violet light) to approximately 700 nm (red light) of the electromagnetic spectrum. In certain embodiments, the light stress is blue light in the visible spectrum from approximately 425 nm to 500 nm, for example, 470 nm. The ultraviolet part of the spectrum (up to approximately 300-400 nm) is divided into three regions: the near ultraviolet, the far ultraviolet, and the extreme ultraviolet. The three regions are distinguished by how energetic the ultraviolet radiation is and by the wavelength of the ultraviolet light, which is related to energy. The near ultraviolet is the light closest to optical or visible light. The extreme ultraviolet is the ultraviolet light closest to X-rays, and is the most energetic of the three types. The far ultraviolet lies between the near and extreme ultraviolet regions.

The source of light may be a fluorescent light, incandescent light, or a light-emitting diode (LED); the light source may be inserted into a tissue culture incubator to provide continuous exposure or to regulate exposure during the time that the retinal cells are cultured. High intensity light sources are useful, providing the capability to apply light at variable intensity levels. LED fixtures can be designed to provide light stress to the cell cultures from above the cell culture plate (which may be any cell culture dish, flask, or multi-well plate) from one LED and below the cell culture plate from a second separate LED. Each LED may emit light of the same intensity or of different intensities, which may be controlled for example by different potentiometers to independently control the current flowing through each LED. The emitted light may be constant, that is, having the same wavelength and intensity over a period of time, or may be cyclical, varying the wavelength or intensity. For example, emitted light that is cyclical may be controlled such that the light stress mimics or matches a circadian rhythm. Light sources that are mounted in a tissue culture incubator can be appropriately placed to ensure proper ventilation such that exposure of the cells to the light source does not result in exposure of the cells or a portion of the cells to changes in temperature.

In another cell culture system, the source of light is a fluorescent light fixture, for example, a set of linear bulbs that provide ambient light to an entire plate, flask, or dish of cells. The bulb may also be large enough to permit exposure of multiple cell culture plates, dishes, or flasks.

The effect of light on retinal cell viability, survival, or neurodegeneration of a retinal neuronal cell in the cell culture may be determined according to methods described herein and practiced in the art. The retinal cell culture light stress model described herein may be used as a model for diseases that affect photoreceptor cells, for example, macular degeneration. For example, the retinal cell culture is exposed to light, particularly blue light, which decreases the survival or kills photoreceptor cells without killing any of the other major retinal cell types that are present in the cell culture system described herein. By way of example, the retinal cell culture system prepared as described herein, when exposed to 6000 lux of white light for 48 hours results in death of photoreceptor cells (over 95%); however, survival of ganglion cells was not reduced or adversely affected.

This model may be also used for studying cellular processes that underlie the pathology of a neurodegenerative diseases or disorders, particularly retinal diseases and disorders. By way of example, light stress affects retinal cells by inducing inappropriate activation of apoptosis (programmed cell death), which can contribute to a variety of pathological disease states. Apoptosis can be determined by a variety of methods known in the art and disclosed herein.

The light stress model may also be useful in a method for determining whether an anti-A2E antibody, or antigen-binding fragment thereof, blocks light from harming the eye. As described in more detail herein, the model may be used in methods for determining that an anti-A2E antibody, or antigen-binding fragment thereof, has the capability to block, inhibit, or prevent light from decreasing survival of retinal cells (e.g., photoreceptor cells) or to decrease or inhibit the progression of or reverse neurodegeneration. The antibody thus acts like a filter at the cellular level to block out harmful light such as ultraviolet or blue light. By way of example, light output applied only above a retinal cell culture and measured below cells that were maintained in culture media containing phenol red (which acts as an acid-base indicator and tints the media red) was 25% less luminous (decreased intensity) than the level of light output measured above the cells. Thus, the red media had a filtering effect that protected photoreceptor cells from the light stress.

Cigarette Smoke Condensate as a Cell Stressor

The retinal cell stressor may be tobacco smoke, one or more compounds found in tobacco smoke, or cigarette smoke condensate. Smoking is believed to be a risk factor for developing macular degeneration (Delcourt et al., Arch. Ophthalmol. 116:1031-35 (1998)). Tobacco smoke contains numerous mutagenic and carcinogenic compounds such as polyaromatic hydrocarbons (PAHs), tobacco-specific nitrosamines (TSNAs), carbazole, phenol, and catechol. PAHs are a group of chemicals in which constituent atoms of carbon and hydrogen are linked by chemical bonds that form two or more rings. Thus PAHs are sometimes called polycyclic hydrocarbons or polynuclear aromatics. Examples of such chemical arrangements are anthracene (3 rings), pyrene (4 rings), benzo(a)pyrene (5 rings), and similar polycyclic compounds. Exposure of bovine retinal pigment epithelial cells to benzo(a)pyrene appeared to inhibit growth and replication of the cells (Patton et al., Exp. Eye Res. 74:513-522 (2002)).

Tobacco specific nitrosamines (TSNAs) are electrophilic alkylating agents that are potent carcinogens. TSNAs are formed by reactions involving free nitrate during processing and storage of tobacco and by combustion of tobacco that contains the alkaloids, nicotine and nomicotine, in a nitrate rich environment. Fresh-cut, green tobacco contains virtually no tobacco specific nitrosamines (see, e.g., U.S. Pat. Nos. 6,202,649 and 6,135,121). In contrast, cured tobacco products obtained according to conventional methods contain a number of nitrosamines, including N′-nitrosonornicotine (NNN) and 4-(N-nitrosomethylamino)-1-(3-pyridyl)-1-butanone (NNK).

Additional toxic compounds produced in cigarette smoke include carbazole, phenol, and catechol. Carbazole is a heterocyclic aromatic compound containing a dibenzopyrrole system and is a suspected carcinogen. The phenolic compounds present in cigarette smoke occur as a result of pyrolysis of the polyphenols chlorogenic acid and rutin. Phenolic compounds in tobacco smoke include catechol, phenol, hydroquinone, resorcinol, o-cresol, m-cresol, and p-cresol. Catechol is the most abundant phenol in tobacco smoke (80-400 μg/cigarette) and has been identified as a co-carcinogen with benzo[a]pyrene.

Cigarette smoke condensate (CSC) may be prepared according to methods described herein and known in the art or may be purchased from a vendor such as Murty Pharmaceuticals (Lexington, Ky.). A mechanical device such as an FTC Smoke Machine or Phipps-Bird 20-channel smoking machine may be used for generating tobacco smoke. Examples of cigarettes used for preparing CSC include 1R4F or 1R3F research cigarettes or the like (see, e.g., Meckley et al., Food Chem. Toxicol. 42:851-63 (2004); Putnam et al., Toxicol. In Vitro 16:599-607 (2002)). To prepare CSC, for example, particulate constituents of tobacco smoke that is generated by one or more cigarettes may be deposited or collected on a filter, such as a glass fiber filter or another filter that is inert during the extraction process. Compounds are extracted from the filters using a solvent, for example, dimethyl sulfoxide (DMSO). The extraction procedure may also include a mechanical force such as sonication that is useful for aiding the removal of the particulate matter from the filters.

The effect of tobacco smoke on survival of retinal cells, particularly retinal neuronal cells, or on neurodegeneration of the retinal neuronal cells, in the presence and absence of an anti-A2E antibody, or antigen-binding fragment thereof, may be determined using the retinal cell culture system described herein. A retinal cell culture may be exposed to cigarette smoke condensate, tobacco smoke, or to one or more constituent compounds of tobacco smoke, including but not limited to the compounds discussed herein. The retinal cells may be exposed to a CSC cell stressor prior to culture of the retinal cells or for a period of time during the culture of the cells. Cells may be exposed to CSC for at least about 3 hours, 6 hours, 9 hours, 12, hours, 18 hours, 24 hours, or 2 days, 3 days, 4 days, 5 days, 6 days, or a week, 2 weeks, 4 weeks, 2 months, 4 months, or longer, or for any period of time between the time periods enumerated. The effect of the cell stressor on cell viability, survival, or alternatively on neurodegeneration, of the retinal cells in the cell culture may be determined according to methods described herein and known in the art.

Cigarette Smoke Condensate Plus Light as a Stressor

A retinal neuronal cell culture may be exposed to more than one cell stressor, for example, the culture may be exposed to at least two retinal cell stressors. For example, one retinal cell stressor may be cigarette smoke condensate and a second cell stressor may be light as described herein.

A retinal neuronal cell culture may be exposed to two cell stressors such as cigarette smoke condensate and a light source, separately or together, and then cultured. Alternatively, the retinal cell culture may be exposed to two cell stressors such as cigarette smoke condensate and a light source, separately or together, during the culture of the retinal neuronal cells. The retinal neuronal cells may be exposed to either one or both of the cell stressors prior to culturing the cells, or the cells may be exposed to one cell stressor prior to culture and then exposed to either one or both of the cell stressors during culture of the cells. The effect of an anti-A2E antibody, or antigen-binding fragment thereof, in the presence of the cell stressors, on the survival or alternatively neurodegeneration of the retinal cells in the cell culture may be determined according to methods described herein and known in the art. The time of exposure of the retinal neuronal cell culture to each cell stressor may differ. Cells may be exposed to CSC and/or light for at least about 3 hours, 6 hours, 9 hours, 12, hours, 18 hours, 24 hours, or 2 days, 3 days, 4 days, 5 days, 6 days, or a week, at least two weeks, and at least one month, or longer, or for any period of time between the time periods enumerated.

As described herein for culture of retinal cells exposed to a light stressor, the light may be emitted from at least one fluorescent light, incandescent light, or at least one light-emitting diode. The exposure may be intermittent or constant, and the duration of exposure may be varied. Alternatively, light stress may be applied as a light shock whereby cells at some point prior to or during cell culture may be protected from exposure to any light source and then exposed to a light stress. The light source may be inserted into a tissue culture incubator to provide continuous exposure or to regulate exposure during the time that the retinal cells are cultured.

The retinal cell culture system described herein may be used as model for diseases that affect photoreceptor cells, for example, macular degeneration. When a light stressor is combined with a CSC stressor, the number of photoreceptor cells that survive is reduced compared to the number of photoreceptor cells that survive when exposed to CSC alone.

The retinal cell culture system comprising a CSC stressor and a light stressor may be also used for studying the effect of an anti-A2E antibody, or antigen-binding fragment thereof, on cellular processes that underlie the pathology of a neurodegenerative disease or disorder, particularly a retinal disease or disorder. For instance, such stressors may affect retinal cells by inducing inappropriate activation of apoptosis (programmed cell death), which can contribute to a variety of pathological disease states. Apoptosis can be determined by a variety of methods known in the art and described herein.

Physical Stressor: Increased Hydrostatic Pressure

The retinal cell stressor may be a physical cell stressor such as elevated hydrostatic pressure (pressure exerted by a liquid, which may be applied by methods described herein and practiced in the art such as, for example, increasing atmospheric pressure). Elevated intraocular pressure (IOP) is known in the art to correlate with glaucoma in patients. Ocular cells exposed to a hydrostatic pressure of 50 mm mercury (Hg) did not appear to have decreased viability, but morphological changes were observed as well as changes in distribution of actin stress fibers in certain cells (see Wax et al., Br. J. Ophthalmol. 84:423-28 (2000)). In one embodiment, the retinal cell culture system comprises isolated mature retinal cells, including retinal neuronal cells, and increased or elevated hydrostatic pressure (or atmospheric pressure) as a cell stressor. Cells may be exposed to a pressure that is 40, 45, 50, 55, 60, 70, 75, 80, 100, 110, 120, or 130 mm Hg (or at any pressure between the mm Hg enumerated). Increased pressure may be applied using methods described herein and known to a skilled artisan, for example, by using a pressure incubator (see, e.g., Healey et al., J. Vasc. Surg. 38:1099-105 (2003)) or by placing a pressure chamber within a tissue culture incubator (see, e.g., Wax et al., supra; see also Vouyouka et al., J. Surg. Res. 110:344-51 (2003)). The retinal neuronal cell culture system may be exposed to increased atmospheric pressure for at least 6 hours, 9 hours, 12, hours, 18 hours, 24 hours, or 2 days, 3 days, 4 days, 5 days, 6 days, or a week, at least two weeks, and at least one month (4 weeks), or longer, or for any period of time between the time periods enumerated.

One or more culture conditions may be adjusted so that the effect of the physical stressor, such as increased hydrostatic pressure, on the retinal cells can be more readily observed. For example, the concentration or percent of fetal bovine serum may be reduced or eliminated from the cell culture when cells are exposed to increased pressure.

In another embodiment, the retinal cell culture system comprises increased hydrostatic pressure (or increased atmospheric pressure) as one cell stressor and a second cell stressor. The retinal neuronal cells may be exposed to increased pressure concomitantly with the second stressor or the cells may be exposed first to one cell stressor and then to the second stressor. In alternative embodiments, the retinal neuronal cells may be exposed to either one or both of the cell stressors prior to culturing the cells; alternatively, the cells may be exposed to one cell stressor prior to culture and then exposed to either one or both of the cell stressors during culture of the cells. The effect of an anti-A2E antibody, or antigen-binding fragment thereof, described herein in the presence of the cell stressors on retinal cell viability, survival, or neurodegeneration of a retinal neuronal cell, may be determined according to methods described herein and known in the art.

Chemical Stressors: Retinoid N-retinylidene-N-retinyl-ethanolamine (A2E) Cell Stressor

Alternatively, the stressor may be a chemical. For example, the chemical stressor may be a vitamin A derivative, such as retinoid N-retinylidene-N-retinyl-ethanolamine (A2E), or a derivative of A2E. A2E stress may include any one or more of A2E isomers including, such as iso-A2E (13-Z photo-isomer of A2E (see, e.g., Parish et al., Proc. Natl. Acad. Sci. USA 95:14609-13 (1998); Ben-Shabat et al., Angew. Chem. int. Ed. 41:814-17 (2002)), or the stress may include all isoforms of A2E. A2E is a component of retinal lipfuscin, which according to non-limiting theory is formed from retinal, digested rhodopsin, and ethanolamine (a cell membrane component), in retinal pigment epithelial cells that line the photoreceptor rods and cones during processing of cellular debris (see, e.g., Parish et al., supra; Mata et al., Proc. Natl. Acad. Sci. USA 97:7154-59 (2000)). Accumulation of A2E has been hypothesized to contribute to development of age-related neurodegeneration of retinal cells, particularly macular degeneration. Exposure of the retinal neuronal cell culture system to A2E results in selective killing of certain cells, particularly photoreceptor cells, that are present in the retinal cell culture system.

The photoreceptors in the retina, designed to initiate the cascade of events that link the incoming light to the sensation of “vision,” are susceptible to damage by light, particularly blue light. The damage can lead to cell death and diseases, particularly the dry form of macular degeneration. The turnover of retinal, an essential element of the visual process, is the basis of the events that lead to damage. Free retinal, absorbing in the blue region of the visible spectrum, is phototoxic and is a precursor of the (photo)toxic compound A2E, which specifically targets cytochrome oxidase and thereby induces cell death by apoptosis.

The retinal cell culture system may be exposed to A2E at any concentration between 1 pM and 200 μM (e.g., 1 pM, 10 pM, 100 pM, 250 pM, 500 pM, 750 pM, 1 nM, 10 nM, 50 nM, 100 nM, 250 nM, 500 nM, 750 nM, 1 μM, 2 μM, 5 μM, 7.5 μM, 10 μM, 15 μM, 20 μM, 25 μM, 40 μM, 50 μM, 75 μM, 100 μM, 120 μM, 200 μM); or 250 μM, 500 μM, or 750 μM), between 1 μM and 40 μM, or between 10 μM and 20 μM, for a period of time, for example, between 2 and 48 hours or between 12 and 36 hours. In another embodiment, the cell culture may be exposed to lower concentrations of A2E (for example, between 1 pM and 10 μM or between 1 nM and 1 μM) for longer times (such as about one week, about two weeks, or about one month (4 weeks)). By way of example, the retinal cell culture system prepared as described herein when exposed to 20 μM A2E for 48 hours results in death of photoreceptor cells (more than 90% of photoreceptor cells die compared to photoreceptor cells not exposed to A2E); survival of ganglion cells is not adversely affected (i.e., ganglion cell viability is not reduced).

As described herein, more than one stressor may be applied to the retinal cell culture system. For example, a culture may be exposed to a light stressor and a chemical stressor such as A2E according to methods and techniques described herein. Additional stressors that are known in the art and described herein, including but not limited to glucose oxygen deprivation, pressure, and neurotoxins, may be combined with either a light stressor or a chemical stressor or both stressors.

Chemical Cell Stressor: Glutamate

A retinal cell culture system may include glutamate as a cell stressor. In the mammalian central nervous system (CNS), the transmission of nerve impulses is controlled by the interaction between a neurotransmitter, which is released by a sending neuron, and a surface receptor on a receiving neuron, which causes excitation of this receiving neuron. Excitatory amino acids (EAAs), principally glutamic acid (the primary excitatory neurotransmitter) and aspartic acid, mediate the major excitatory pathway in the mammalian central nervous system. Thus, glutamic acid can bring about changes in the postsynaptic neuron that reflect the strength of the incoming neural signals. The receptors that respond to glutamate are called excitatory amino acid receptors (EAA receptors) (see, e.g., Watkins et al., Trans. Pharm. Sci. 11:25 (1990); Monaghan et al., Annu. Rev. Pharmacol. Toxicol. 29:365 (1989); Watkins et al., Annu. Rev. Pharmacol. Toxicol. 21:165 (1981)). The excitatory amino acids play a role in a variety of physiological processes, such as long-term potentiation (learning and memory), the development of synaptic plasticity, motor control, respiration, cardiovascular regulation, and sensory perception.

Excitatory amino acid receptors are classified into two general types: ionotropic and metabotropic. The ionotropic receptors contain ligand-gated ion channels and mediate ion fluxes for signaling, while the metabotropic receptors use G-proteins for signaling. Both types of receptors appear not only to mediate normal synaptic transmission along excitatory pathways, but also to participate in the modification of synaptic connections during development and throughout life (see, e.g., Schoepp et al., Trends in Pharmacol. Sci. 11:508 (1990); McDonald et al., Brain Res. Rev. 15:41 (1990)).

Further sub-classification of the ionotropic EAA glutamate receptors is based upon the agonists (stimulating agents) other than glutamic and aspartic acid that selectively activate the receptors. The at least three subtypes of the ionotropic receptors are defined by the depolarizing actions of allosteric modulators: a receptor responsive to N-methyl-D-aspartate (NMDA); a receptor responsive to alpha-amino-3-hydroxy-5-methylisoxazole-4-propionic acid (AMPA); and a receptor responsive to kainic acid (KA). The NMDA receptor controls the flow of both divalent (Ca⁺⁺) and monovalent (Na⁺, K⁺) ions into the postsynaptic neural cell. The AMPA and KA receptors also regulate the flow into postsynaptic cells of monovalent K⁺ and Na⁺, and occasionally divalent calcium (Ca⁺⁺). Other glutamate agonists in addition to NMDA, AMPA, and KA include aspartate, ACPD, quisqualic acid, ibotenic acid, and quinolinic acid. A glutamate agonist may be included as a retinal cell stressor in the mature retinal cell culture system at concentrations and for a duration and at times as described herein for the inclusion of glutamate as a cell stressor.

The G-protein excitatory amino acid receptor is coupled to multiple second messenger systems that lead to enhanced phosphoinositide hydrolysis, activation of phospholipase D, increased or decreased c-AMP formation, and/or changes in ion channel function (see, e.g., Schoepp et al., Trends in Pharmacol. Sci. 14:13 (1993)). The metabotropic EAA receptors are divided into three sub-groups, which are unrelated to ionotropic receptors, and are coupled via G-proteins to intracellular second messengers. These metabotropic EAA receptors are classified based on receptor homology and second messenger linkages. EAA receptors have been implicated during development in specifying neuronal architecture and synaptic connectivity and may be involved in experience-dependent synaptic modifications.

These receptors appear to be involved in a broad spectrum of CNS disorders. For example, during brain ischemia caused by stroke or traumatic injury, excessive amounts of the EAA glutamic acid are released from damaged or oxygen-deprived neurons. Binding of this excess glutamic acid to the postsynaptic glutamate receptors opens their ligand-gated ion channels, thereby allowing an ion influx that in turn activates a biochemical cascade resulting in protein, nucleic acid, and lipid degradation, and cell death. This phenomenon, known as excitotoxicity, may also be responsible for the neurological damage associated with other disorders ranging from hypoglycemia, ischemia, and epilepsy to chronic neurodegeneration that occurs in Huntington's, Parkinson's, and Alzheimer's diseases (see, e.g., Kannurpatti et al., Neurochem. Int. 44:361-69 (2004); Curr. Top. Med. Chem. 4:149-77 (2004); Swanson et al., Curr. Mol. Med. 4:193-205 (2004)). Excessive activation of ionotropic receptors and group I metabotropic receptors may result in neuronal death. Many neurodegenerative conditions, including Parkinson's disease, Alzheimer's disease, cerebral ischaemia, epilepsy, Huntington's chorea and amyotrophic lateral sclerosis (ALS), have been linked to disturbed glutamate homeostasis (Tortarolo et al., J. Neurochem. 88:481-93 (2004); Lipton et al, New Eng. J. Med. 330:613-22 (1994); Gegelashvili et al., Mol. Pharmacol. 52:6-15 (1997); Robinson et al., Adv. Pharmacol. 37:69-115 (1997)).

In glaucoma, the increased release of glutamate is a major cause of retinal ganglion cell death (see, e.g., El-Remessy et al., Am. J. Pathol. 163:1997-2008 (2003)). Extracellular glutamate concentrations are maintained within physiological levels exclusively by glutamate transporters, permitting normal excitatory transmission as well as protecting against excitotoxicity (Robinson et al., Adv. Pharmacol. 37:69-115 (1997)). Nerve damage may be caused by abnormal accumulation of glutamate that leads to overexcitation of the receiving nerve cell or may be caused by oversensitive glutamate receptors on the receiving nerve cell.

The cell culture system described herein comprising mature retinal cells including retinal neuronal cells for determining the effect of an anti-A2E antibody, or antigen-binding fragment thereof, on retinal cell survival or cell viability may comprise glutamate or a derivative thereof (see, e.g., U.S. Patent Application No. 2002/0115688) or a glutamate agonist as a cell stressor (see Luo et al., supra). The concentration of glutamate added to a retinal cell culture may be between 0.5 nM-100 μM, such as about 0.5 nM, 1 nM, 2 nM, 4 nM, 5 nM, 7.5 nM, 10 nM, 20 nM, 40 nM, 50 nM, 75 nM, 100 nM, 0.1 μM, 0.5 μM, 1 μM, 2 μM, 4 μM, 5 μM, 7.5 μM, 10 μM, 20 μM, 25 μM, 40 μM, 50 μM, 60 μM, 75 μM, or 100 μM, or between 100 μM and 1 mM, such as about 150 μM, 200 μM, 250 μM, 300 μM, 400 μM, 500 μM, 600 μM, 750 μM, 800 μM, 900 μM, and 1000 μM (1 mM). Glutamate acting as a cell stressor may be added to a retinal cell culture at the time the freshly harvested (isolated) retinal cells are prepared and plated for tissue culture. Alternatively, glutamate may be added at a time subsequent to plating and establishment of the retinal cells in culture. Glutamate may be added one day after plating the retinal cells, two days, three days, four days, five days, six days, or 7 days (one week), 2 weeks, 3 weeks, 4 weeks, or 6 weeks, or longer, after plating of the cells.

Glutamate may also be combined with one or more other cell stressors described herein, for example, light stress, CSC, A2E stress, or increased hydrostatic pressure. As described herein when a retinal cell culture is exposed to two or more cell stressors, glutamate and one or more other cell stressors may be applied or added to the cell culture together at the same time or may be applied or added to the cell culture separately at different times and in any order. The time of exposure to each cell stressor may be different or may be the same.

A glutamate stress retinal cell culture model with or without additional cell stressors may be used. Neurodegeneration may be affected any one of a number of different pathways and receptors that are affected by excitotoxic mechanisms. For example, activation of glutamate receptors can trigger death of neurons and some types of glial cells, particularly when cells are also subjected to adverse conditions such as reduced levels of oxygen or glucose, increased levels of oxidative stress, exposure to toxins, or a genetic mutation. Excitotoxic death that occurs as a result of one or more of these adverse conditions may involve excessive calcium influx, release of calcium from internal cell organelles, radical oxygen species production, and engagement of apoptotic cascades. See, e.g., Mattson, Neuromolecular Med 3:65-94 (2003); Atlante et al., FEBS Lett. 497:1-5 (2001).

Assay Methods

In one embodiment, the cell culture system and the in vitro retinal cell culture stress model described herein is used for biological testing of an anti-A2E antibody, or antigen-binding fragment thereof, described herein, that may be suitable for treatment of neurological diseases or disorders in general, and for treatment of degenerative diseases of the eye and brain in particular. Methods for altering (increasing or decreasing in a statistically significant manner) viability of a mature retinal cell comprise contacting (combining, mixing, or otherwise permitting interaction of) an anti-A2E antibody, or antigen-binding fragment thereof, with the mature retinal cells present in a retinal cell culture system (in the absence or presence of one or more cell stressors) under conditions and for a time sufficient to permit interaction between the an anti-A2E antibody, or antigen-binding fragment thereof, and the cell culture system, and then comparing the viability of a plurality of mature retinal cells in the presence of the antibody with the viability of a plurality of mature retinal cells in the absence of the antibody. The plurality of retinal cells that are not exposed to the anti-A2E antibody, or antigen-binding fragment thereof, may be prepared simultaneously from the same retinal tissue as the retinal cells that are exposed to the antibody. Alternatively, the viability of retinal cells in the presence of the anti-A2E antibody, or antigen-binding fragment thereof, may be quantified and compared to viability of a standard retinal cell culture (i.e., a retinal cell culture system as described herein that provides repeatedly consistent, reliable, and precise determinations of retinal cell viability).

In a particular embodiment, a method is provided for enhancing survival of a retinal neuronal cell (which may be a photoreceptor cell, amacrine cell, ganglion cell, horizontal cell, and/or a bipolar cell) wherein the method comprises contacting (combining, mixing, or otherwise permitting interaction of) an anti-A2E antibody, or antigen-binding fragment thereof, with the mature retinal cells present in a retinal cell culture system (in the absence or presence of one or more cell stressors) under conditions and for a time sufficient to permit interaction between the anti-A2E antibody, or antigen-binding fragment thereof, and the cell culture system, and then comparing survival of a plurality of mature retinal cells in the presence of the antibody with the survival of a plurality of mature retinal cells in the absence of the antibody. As described herein, the plurality of retinal cells that are not exposed to the anti-A2E antibody, or antigen-binding fragment thereof, may be prepared simultaneously from the same retinal tissue as the retinal cells that are exposed to the antibody. Alternatively, survival of retinal cells in the presence of the anti-A2E antibody, or antigen-binding fragment thereof, may be quantified and compared to survival of a standard retinal cell culture (i.e., a retinal cell culture system as described herein that provides repeatedly consistent, reliable, and precise determinations of retinal cell survival and viability).

In one embodiment, an anti-A2E antibody, or antigen-binding fragment thereof, is incorporated into screening assays comprising the retinal cell culture stress model system to determine whether the antibody is capable of altering neurodegeneration of neuronal cells (impairing, inhibiting, preventing, abrogating, reducing, slowing the progression of, or accelerating in a statistically significant manner). A preferred antibody or antigen-binding fragment thereof inhibits, reduces, abrogates, slows the progression of, or impairs neurodegeneration of a neuronal cell, particularly a retinal neuronal cell, is capable of regenerating a neuronal cell, and/or is capable of enhancing or prolonging survival (promoting, improving, or increasing survival, thus delaying injury and/or death) of a neuronal cell. An anti-A2E antibody, or antigen-binding fragment thereof, that inhibits neurodegeneration of a neuronal cell may be identified by contacting (mixing, combining, or otherwise permitting interaction between the antibody, or antigen-binding fragment thereof, and retinal cells of the cell culture system) with the cell culture system under conditions and for a time sufficient to permit interaction between the anti-A2E antibody, or antigen-binding fragment thereof, and the retinal cells, particularly the mature retinal neuronal cells of the cell culture system described herein.

An anti-A2E antibody, or antigen-binding fragment thereof, may act directly upon a retinal neuronal cell in a manner that affects survival or neurodegeneration (or neuronal cell injury) of the cell. Alternatively, the anti-A2E antibody, or antigen-binding fragment thereof, may act indirectly by interacting with one retinal cell type that consequently, via a biological response to the compound, affects viability, that is survival and/or neurodegeneration, of another retinal cell. Not wishing to be bound by theory, the capability of an A2E specific antibody to inhibit, prevent the accumulation of, or decrease A2E in a cell or in the extracellular environment by binding to A2E may enhance survival, increase retinal cell viability, and/or minimize or decrease neurodegeneration.

In certain embodiments, the methods described herein may be used for identifying or determining that an anti-A2E antibody, or antigen-binding fragment thereof, alters viability (i.e., alters survival and/or neurodegeneration and/or neuronal cell injury) of one, two, three, or more, or all retinal cell types and may also be used to identify and/or determine that the antibody alters viability of one, two, three, or more, or all retinal neuronal cell types (amacrine cell, a photoreceptor cell, a ganglion cell, horizontal cell, and bipolar cell). In certain other embodiments, the screening methods may be used to identify and/or determine that an anti-A2E antibody, or antigen-binding fragment thereof, alters viability (preferably enhances survival and/or inhibits neurodegeneration or cell injury) of one retinal neuronal cell type, such as an amacrine cell, a photoreceptor cell, or a ganglion cell, horizontal cell, or bipolar cell.

In other embodiments, an antibody specific for A2E (or antigen-binding fragment thereof) may be used in a method, such as a competition assay, in which candidate agents are screened to identify an agent that will compete with the antibody for binding to A2E. The interaction between an antibody and A2E may be determined using immunoassay methods known in the art and described herein. Such a candidate agent may be useful for enhancing retinal cell survival or increasing retinal cell viability and thus useful for treating and/or preventing macular degeneration.

An anti-A2E antibody, or antigen-binding fragment thereof, as described herein, that effectively inhibits neurodegeneration of a neuronal cell, particularly a retinal neuronal cell, may be identified and/or evaluated for its ability to enhance retinal neuronal cell survival by techniques known in the art and described herein for determining the effects of the agent on retinal neuronal cell structure or morphology; expression of retinal neuronal cell markers (e.g., β3-tubulin, rhodopsin, recoverin, visinin, calretinin, calbindin, Thy-1, tau, microtubule-associated protein 2, and the like (see, e.g., Espanel et al., Int. J. Dev. Biol. 41:469-76 (1997); Ehrlich et al., Exp. Neurol. 167:215-26 (2001); Kosik et al., J. Neurosci. 7:3142-53 (1987)); and cell survival (i.e., cell viability or length of time until cell death). Preferably, the an anti-A2E antibody, or antigen-binding fragment thereof, enhances survival of neuronal cells such as retinal neuronal cells and more particularly photoreceptor cells, that is, the agent promotes survival or prolongs survival such that the time period in which neuronal cells are viable is extended.

In another embodiment, an anti-A2E antibody, or antigen-binding fragment thereof, is incorporated into screening assays comprising the retinal cell culture stress model system described herein to determine whether the antibody increases viability (i.e., increases in a statistically significant or biologically significant manner) of a plurality of retinal cells. A person skilled in the art would readily appreciate and understand that as described herein a retinal cell that exhibits increased viability means that the length of time that a retinal cell survives in the cell culture system is increased (increased lifespan) and/or that the retinal cell maintains a biological or biochemical function (normal metabolism and organelle function; lack of apoptosis; etc.) compared with a retinal cell cultured in an appropriate control cell system (e.g., the cell culture system described herein in the absence of the compound). Increased viability of a retinal cell may be indicated by delayed cell death or a reduced number of dead or dying cells; maintenance of structure and/or morphology; lack of or delayed initiation of apoptosis; delay, inhibition, slowed progression, and/or abrogation of retinal neuronal cell neurodegeneration or delaying or abrogating or preventing the effects of neuronal cell injury. Methods and techniques for determining viability of a retinal cell and thus whether a retinal cell exhibits increased viability are described in greater detail herein and are known to persons skilled in the art.

In certain embodiments, a method is provided for determining whether an anti-A2E antibody, or antigen-binding fragment thereof, enhances survival of photoreceptor cells. One method comprises contacting a retinal cell culture system as described herein with the agent under conditions and for a time sufficient to permit interaction between the retinal neuronal cells and the compound. Enhanced survival (prolonged survival) may be measured according to methods described herein and known in the art, including detecting expression of rhodopsin. Rhodopsin, which is composed of the protein opsin and retinal (a vitamin A form), is located in the membrane of the photoreceptor cell in the retina of the eye and catalyzes the only light sensitive step in vision. The 11-cis-retinal chromophore lies in a pocket of the protein and is isomerised to all-trans retinal when light is absorbed. The isomerisation of retinal leads to a change of the shape of rhodopsin, which triggers a cascade of reactions that lead to a nerve impulse that is transmitted to the brain by the optical nerve.

The capability of an anti-A2E antibody, or antigen-binding fragment thereof, to increase retinal cell viability and/or to enhance, promote, or prolong cell survival (that is, to extend the time period in which retinal neuronal cells are viable), and/or impair, inhibit, or impede neurodegeneration as a direct or indirect result of the herein described stress may be determined by any one of several methods known to those skilled in the art. For example, changes in cell morphology in the absence and presence of an anti-A2E antibody, or antigen-binding fragment thereof, may be determined by visual inspection such as by light microscopy, confocal microscopy, or other microscopy methods known in the art. Survival of cells can also be determined by counting viable and/or nonviable cells, for instance. Immunochemical or immunohistological techniques (such as fixed cell staining or flow cytometry) may be used to identify and evaluate cytoskeletal structure (e.g., by using antibodies specific for cytoskeletal proteins such as glial fibrillary acidic protein, fibronectin, actin, vimentin, tubulin, or the like) or to evaluate expression of cell markers as described herein. The effect of an anti-A2E antibody, or antigen-binding fragment thereof, on cell integrity, morphology, and/or survival may also be determined by measuring the phosphorylation state of neuronal cell polypeptides, for example, cytoskeletal polypeptides (see, e.g., Sharma et al., J. Biol. Chem. 274:9600-06 (1999); Li et al., J. Neurosci. 20:6055-62 (2000)). Cell survival or, alternatively cell death, may also be determined according to methods described herein and known in the art for measuring apoptosis (for example, annexin V binding, DNA fragmentation assays, caspase activation, marker analysis, e.g., poly(ADP-ribose) polymerase (PARP), etc.).

Enhanced survival (or prolonged or extended survival) of one or more retinal cell types in the presence of an anti-A2E antibody, or antigen-binding fragment thereof, indicates that the antibody may be an effective agent for treatment of a neurodegenerative disease, particularly a retinal disease or disorder. Cell survival and enhanced cell survival may be determined according to methods described herein and known to a skilled artisan including viability assays and assays for detecting expression of retinal cell marker proteins. For determining enhanced survival of photoreceptor cells, opsins may be detected, for instance, including the protein rhodopsin that is expressed by rods. Rhodopsin, which is composed of the protein opsin and retinal (a vitamin A form), is located in the membrane of the photoreceptor cell in the retina of the eye and catalyzes the only light sensitive step in vision. The 11-cis-retinal chromophore lies in a pocket of the protein and is isomerized to all-trans retinal when light is absorbed. The isomerization of retinal leads to a change of the shape of rhodopsin, which triggers a cascade of reactions that lead to a nerve impulse that is transmitted to the brain by the optical nerve.

Viability (or survival) of one or more retinal cell types that are present in the cell culture system described herein may be determined according to methods described herein and which are familiar to a skilled artisan. For example, viable cells may be differentiated from non-viable cells by uptake of particular dyes, such as trypan blue. Alternatively, cell death and cell lysis may be quantified by measuring cellular metabolites or enzymes, such as alkaline and acid phosphatase, glutamate-oxalacetate transaminase, glutamate pryuvate transaminase, argininosuccinate lyase, and lactate dehydrogenase, that are released into cell culture media supernatant from the damaged cells (e.g., via damaged or compromised plasma membranes) or upon cell expiration. For example, viability assays may be employed that use esterase substrates, stain nucleic acids, or that measure oxidation or reduction (see Molecular Probes, Eugene, Oreg., Invitrogen Life Sciences, Carlsbad, Calif.). Viability of living cells that are not actively dividing, such as retinal neuronal cells, may be determined by evaluating one or more metabolic processes. Such methods incorporate reagents that may be detected by calorimetric or fluorimetric analyses. Companies that provide assay kits for determining cell viability/vitality or cytotoxicity include Roche Applied Science, Indianapolis, Ind. and Molecular Probes.

Viability of one or more retinal cell types in the cell culture system may be determined by assessing survival of the one, two, three, or more retinal cell types. Viability or survival of retinal cells in the cell culture system in the absence or presence of one or more cell stressors may be determined, as well as viability (survival) in the absence or presence of an anti-A2E antibody, or antigen-binding fragment thereof. In a preferred embodiment, the anti-A2E antibody, or antigen-binding fragment thereof, enhances or prolongs survival of one or more retinal cell types. Survival may be determined by comparing the number (or percent) of retinal cells exposed to the anti-A2E antibody, or antigen-binding fragment thereof, that are viable over a defined period of time relative to the number (or percent) of retinal cells not exposed to the anti-A2E antibody, or antigen-binding fragment thereof, that are viable over the same defined time period. Survival of retinal cells in the cell culture system may be compared during the time the cells are exposed to the anti-A2E antibody, or antigen-binding fragment thereof, or may be compared for a period(s) of time after the anti-A2E antibody, or antigen-binding fragment thereof, is removed from the cell culture system. The time period may be 1 day, 2-3 days, 4-7 days, 7-14 days, or 14-28 days, 2 months, 4, months, or longer.

To determine that an anti-A2E antibody, or antigen-binding fragment thereof, effectively alters, preferably inhibits, impairs, slows the progression of, prevents, decreases, or reverses neurodegeneration or neuronal cell injury of a retinal neuronal cell may be accomplished by techniques known in the art and described herein for determining the effects of the anti-A2E antibody, or antigen-binding fragment thereof, on neuronal cell structure or morphology; expression of neuronal cell markers (e.g., β3-tubulin, rhodopsin, recoverin, visinin, calretinin, calbindin, neurofilament (NFM), Thy-1, tau, microtubule-associated protein 2, neuron-specific enolase, protein gene product 95, and the like (see, e.g., Espanel et al., Int. J. Dev. Biol. 41:469-76 (1997); Ehrlich et al., Exp. Neurol. 167:215-26 (2001); Kosik et al., J. Neurosci. 7:3142-53 (1987); Luo et al., supra)); and/or cell survival (i.e., cell viability or length of time until cell death). Antibodies that may be used include antibodies that specifically bind to a protein that is expressed by specific cell types (e.g., opsins expressed by photoreceptor cells, for example, rhodopsin expressed by rods; β3-tubulin expressed by interneurons and ganglion cells; and NFM expressed by ganglion cells), and include antibodies that specifically identify a cell marker expressed by a retinal cell from a specific animal source.

An anti-A2E antibody, or antigen-binding fragment thereof, may affect regeneration of retinal neuronal cells. Regeneration of neuronal cells or proliferation of neuronal cells may be determined by any of several methods known in the art, for example, by measuring incorporation of labeled deoxyribonucleotides or ribonucleotides or derivatives thereof, such as tritiated thymidine, or such as by measuring incorporation of bromodeoxyuridine (BrdU), which can be detected by using antibodies that specifically bind to BrdU.

Viability, cell survival or, alternatively cell death, may also be determined according to methods described herein and known in the art for determining whether cells are apoptotic (for example, annexin V binding, DNA fragmentation assays (such as terminal deoxynucleotide transferase-mediated dUTP nick-end labeling (TUNEL)); caspase activation; mitochondrial membrane potential breakdown; marker analysis, e.g., poly(ADP-ribose) polymerase (PARP); detection with antibodies specific for enzymes or polypeptides expressed during apoptosis (e.g., an anti-caspase-3 antibody; etc.).

The disclosed methods and cell culture model systems permit precise measurements of specific interactions occurring between neurons, as well as enabling detailed analysis of subtleties in neuron structure. For instance, the methods and cultured cells described herein are compatible with neurochips, cell-based biosensors, and other multielectrode or electrophysiologic devices for stimulating and recording data from cultured neurons (see, for instance, M. P. Maher et al., J. Neurosci. Meth. 87:45-56, 1999; K. H. Gilchrist et al., Biosensors & Bioelectronics 16:557-64, 2001).

Through use of the methods described herein, an anti-A2E antibody, or antigen-binding fragment thereof, may be tested to identify and/or confirm that the compounds are useful for treating diseases and disorders of the central nervous system and retina, including but not limited to neurodegenerative diseases, epilepsy, glaucoma, macular degeneration, diabetic retinopathy, retinal detachment, retinal blood vessel (artery or vein) occlusion, retinitis pigmentosa, inflammatory retinal diseases, optic neuropathy, and retinal disorders associated with other degenerative diseases such as Alzheimer's disease, Parkinson's disease, or multiple sclerosis, or associated with AIDS. The cultured mature neurons provided herein are particularly useful for screening compounds and molecules such as an anti-A2E antibody, or antigen-binding fragment thereof, to identify an agent that may enable or effect regeneration of retinal tissue that has been damaged by disease. For example, the presence of photoreceptors with an intact outer segment is relevant in such an assay to identify compounds useful for treating neurodegenerative eye diseases.

As described herein, methods are provided for determining the capability of a molecule such as an anti-A2E antibody, or antigen-binding fragment thereof, to alter viability of a retinal cell, which methods include light as a cell stressor. In another embodiment, A2E is added as a cell stressor. Such methods may include more than one cell stressor. For example, the light plus cigarette smoke condensate stress model may be used to determine the capability of the antibody to impair or inhibit the activity of A2E such that A2E is inhibited or blocked from acting as a stressor in the retinal cell culture system. As described herein, A2E is a component of retinal lipofuscin, which as described herein and according to non-limiting theory is formed from retinal, digested rhodopsin, and ethanolamine (a cell membrane component), in retinal pigment epithelial cells that line the photoreceptor rods and cones during processing of cellular debris (see, e.g., Parish et al., supra; Mata et al., Proc. Natl. Acad. Sci. USA 97:7154-59 (2000)). Accumulation of A2E may play some role in development of age-related neurodegeneration of retinal cells, particularly macular degeneration. Exposure of the retinal cell culture system described herein to A2E results in selective killing of certain cells, particularly photoreceptor cells, that are present in the retinal cell culture.

Methods for Detecting A2E in a Biological Sample

In one embodiment, the antibodies (or antigen-binding fragments thereof) are used for binding, removing, quantifying, and/or generally detecting the presence of A2E in a biological sample. The antibodies disclosed herein are therefore useful for diagnosis and prognosis of macular degeneration and other ophthalmic diseases. Any number of assays may be used for detecting A2E and include, for example, an ELISA, immunofluorescence (e.g., immunohistochemistry), immunoprecipitation, immunoblotting, countercurrent immunoelectrophoresis, radioimmunoassays, dot blot assays, chemiluminiscent assay, bioluminescent assay, surface plasmon resonance, inhibition or competition assays, and the like, which may be readily performed by those having ordinary skill in the art (see, e.g., Amara et al., Neurosci. Lett. 185:147-50 (1995); Brand et al., Planta Med. 70:986-92 (2004)). The methods described herein using at least one anti-A2E antibody provide greater sensitivity of detection than currently used biochemical methods such as high pressure liquid chromatography (HPLC) (see, e.g., Parish et al., Proc. Natl. Acad. Sci. USA 95:14609-13 (1998)).

As described herein, autofluorescent pigments such as A2E that accumulate in RPE cells have been implicated in the etiology of ophthalmic diseases, such as macular degeneration (including juvenile macular degeneration such as Stargardt's disease). Accordingly, the anti-A2E antibodies described herein may be used for determining whether a subject has developed a retinal disease such as macular degeneration or is at risk for developing a retinal disease or disorder.

A biological sample may be provided by obtaining a blood sample (from which serum or plasma may be prepared), biopsy specimen, tissue explant, organ culture, or any other tissue or cell preparation from a subject or a biological source. A sample may further refer to a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. The subject or biological source may be a human or non-human animal, a primary cell culture, or culture adapted cell line. In particular embodiments, the biological sample is obtained from the eye of a subject and includes but is not limited to blood, serum, vitreous fluid, aqueous humor, intraocular fluid, and tears.

The presence of A2E may be detected in a biological sample by contacting (i.e., combining, mixing, or otherwise permitting interaction of) the sample with an antibody, or antigen-binding fragment thereof, that binds specifically to A2E under conditions and for a time sufficient to permit formation of an antibody-A2E complex, and then detecting the complex (i.e., detecting a level of antibody/A2E complex). The complex may be detected by adding a reagent that detects the anti-A2E antibody, such as an immunoglobulin-specific reagent (such as an anti-constant region antibody) that is detectably labeled (referred to as a secondary reagent or secondary antibody). Detecting a level of antibody/A2E complex includes detection of the presence of A2E that is above background, which is determined by including the appropriate controls for the particular type of assay with which a skilled artisan is very familiar. The level of antibody/A2E complex may also be quantified, for example, by comparing the level of antibody/A2E complex detected in a sample with detection of a known amount of A2E by the antibody (i.e., comparing the level of complex detected in a sample to the level of A2E/A2E antibody complex using known amounts of A2E to establish a standard curve). Either the anti-A2E antibody or, if used, the secondary reagent may be labeled with or conjugated to an enzyme, such as horseradish peroxidase, glucose oxidase, urease or alkaline phosphatase, that generates a colored product upon contact with a chromogenic substrate or a binding ligand such as biotin and/or avidin or streptavidin compounds. Alternatively, the anti-A2E antibody or secondary reagent may be attached or conjugated according to methods practiced in the art to a radionuclide or a fluorophore (any number of fluorophores may be used and which are available from commercial vendors (see, for example, Molecular Probes, Eugene, Oreg.)). (See, e.g., U.S. Pat. Nos. 3,817,837; 3,850,752, 3,939,350, 3,996,345; 4,277,437; 4,275,149, 4,366,241; Harlow and Lane, supra).

Such methods for detecting the presence of A2E in a biological sample may include any one of a number of immunoassay formats. For example, the biological sample suspected of containing A2E can be immobilized on a solid support (such as but not limited to a microtiter well plate, a glass slide, nitrocellulose, polyvinylidene fluoride membrane and the like). Prior to the sample being immobilized on a solid support, the sample may be subjected (but not necessarily) to methods for removing partially or in total non-A2E molecules and macromolecules and/or cells or cellular material. An antibody that specifically binds to A2E may then be contacted and incubated with the bound sample, and A2E detected by determining the presence of A2E/anti-A2E antibody complexes as described herein.

In an alternative immunoassay format, sometimes referred to as a capture immunoassay, an antibody that specifically binds to A2E (or an antigen-binding fragment thereof) may be bound to a solid support to which the sample is added to permit contact between the anti-A2E antibody and the sample. A2E that is present in the sample and that binds to the immobilizing anti-A2E antibody may be detected by contacting a second anti-A2E antibody that binds to a different A2E epitope than the first antibody. The second anti-A2E antibody may have a detectable label, or the presence of a first anti-A2E antibody/A2E/second anti-A2E antibody complex may be detected with a secondary reagent or secondary antibody that does not bind to the antibody that is immobilized on the solid surface. For example, the immobilized anti-A2E antibody and the second anti-A2E antibody may have constant regions of different immunoglobulin classes or of different species such that a secondary antibody may be used that is specific for the particular constant region of the second anti-A2E antibody. According to still another immunoassay format, sometimes referred to as a competitive or inhibition immunoassay, detectably labeled A2E of known amounts is permitted to compete with A2E present in a sample for binding to an antibody that specifically binds to A2E.

Another immunoassay for which anti-A2E antibodies may be used is immunohistochemistry that may be performed for detecting the presence of A2E in a biological sample, such as a cellular or tissue exudates or a biopsy sample or any other sample that contains retinal neuronal cells or RPE cells. A tissue sample may be fresh-frozen and/or formalin-fixed using paraformaldehyde, according to methods practiced in the art. By way of example, the tissue samples (or sections) may then be contacted with an anti-A2E antibody, or fragment thereof, which may be conjugated to a fluorophore that can be detected by visualization with a microscope. Alternatively, the antibody or antigen-binding fragment may be conjugated or attached to biotin and the A2E-antibody/biotin complex detected with an avidin or streptavidin reagent (e.g., avidin or streptavidin conjugated, for example, to an enzyme such as horseradish peroxidase or alkaline phosphatase according to methods routinely practiced by persons skilled in the art).

As described herein, the A2E specific antibody or the second antibody or reagent may contain a detectable reporter moiety or label such as an enzyme, dye, radionuclide, luminescent group, fluorescent group, or biotin, or the like. The amount of the A2E specific antibody or second reagent that binds to A2E or to the A2E antibody, respectively, is determined using a method appropriate for the specific detectable reporter moiety or label. For radioactive groups, scintillation counting or autoradiographic methods are generally appropriate. Antibody-enzyme conjugates may be prepared using a variety of coupling techniques (for review see, e.g., Scouten, W. H., Methods in Enzymology 135:30-65 (1987)). Spectroscopic methods may be used to detect dyes (including, for example, colorimetric products of enzyme reactions), luminescent groups, and fluorescent groups. Biotin may be detected using avidin or streptavidin, coupled to a different reporter group (commonly a radionuclide, fluorescent group, or an enzyme). Enzyme reporter groups may generally be detected by the addition of substrate (generally for a specific period of time), followed by spectroscopic, spectrophotometric, or other analysis of the reaction products. Standards and standard additions may be used to determine the level of A2E in a sample, using well known techniques.

In another embodiment of the invention, a kit is provided for use according to any of the above detection methods. Such kits typically comprise one or more components necessary for performing an assay for detecting the presence of A2E in a biological sample. Components may be compounds, including an A2E antibody or antibodies or fragments thereof; reagents; containers; and/or equipment. For example, one container within a kit may contain a monoclonal antibody or fragment thereof that specifically binds to A2E. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Treatment of Neurodegenerative Diseases

In another embodiment, methods are provided for treating and/or preventing neurodegenerative diseases and disorders, particularly neurodegenerative retinal diseases and ophthalmic diseases as described herein. A subject in need of such treatment may be a human or non-human primate or other animal who has developed symptoms of a neurodegenerative retinal disease or who is at risk for developing a neurodegenerative retinal disease. As described herein, a method is provided for enhancing survival of neuronal cells such as retinal neuronal cells, particularly photoreceptor cells, by administering an antibody that specifically binds to A2E, or an antigen-binding fragment of the antibody, to a subject having a neurodegenerative retinal disease a composition comprising the antibody and a pharmaceutically acceptable excipient or carrier (that is a pharmaceutically acceptable vehicle, diluent, etc.). An antibody may be suitable for treatment of neurological diseases or disorders in general (see, e.g., U.S. Pat. No. 6,703,015; Janus, CNS Drugs 17:457-74 (2003); Chauhan et al., Neurosci. Lett. 375:143-47 (2005)) and for treatment of degenerative diseases of the eye and brain in particular.

In a certain other embodiment, methods are provided for treating and/or preventing a neurodegenerative retinal disease (an ophthalmic disease) in a subject by administering an A2E conjugate as described herein. As described herein the A2E conjugate may be used to induce an immune response such that the A2E molecule will be recognized as a foreign or non-self antigen (i.e., acquired immunity). Typically, more than one immunization with an antigen is required to improve and/or sustain the duration and/or quality (e.g., affinity of an antibody for A2E) of immune responses directed at A2E. A composition that comprises an A2E conjugate used for inducing an immune response, that is, immunizing a subject or host may be combined with an adjuvant, which may potentiate or enhance the immune response. An adjuvant may be combined with an A2E conjugate to enhance long term release of A2E, which increases the length of time that A2E is presented to the host immune system for processing as well as increase the duration of the antibody response. An adjuvant may also act as a non-specific mediator of immune cell function by stimulating or modulating immune cells. An adjuvant may also enhance macrophage phagocytosis after binding of the antigen as a particulate. Aluminum salt adjuvants (e.g., aluminum hydroxide, aluminum phosphate, and other aluminum salts referred to as alum) and calcium phosphate are most typically used as adjuvants in vaccines administered to humans. To stimulate the ocular mucosal immune system, synthetic immunostimulatory oligodeoxynucleotides that contain unmethylated CpG motifs may be combined with an A2E conjugate (see, e.g., Nesburn et al., Vaccine 23:873-83 (2005); Klinman et al., Immunol. Rev. 199:201-16 (2004)). Additional adjuvants monophosphoryl lipid A (MPL®) (a derivative of lipid A from Salmonella minnesota, Corixa Corp., Hamilton, Mont.) has been tested in humans; other synthetic lipid A mimetics include aminoalkyl glucosaminide 4-phosphates (see, e.g, Baldridge et al., Expert Opin. Biol. Ther. 4:1129-38 (2004)). Additional adjuvants include immune-stimulating complexes (ISCOMs), which are antigen-modified saponin/cholesterol micelles (for example, ISCOMATRIX®(CSL Limited, Australia); Quil A, a highly refined form of saponin, which may also be mixed with cholesterol to form an ISCOM; and QS-21 (Antigenics, New York, N.Y.) (see, e.g., Pearse et al., Vaccine 22:2391-95 (2004); Stewart et al., Vaccine 22:3738-43 (2004) see also, e.g., U.S. Pat. No. 5,773,007). To stimulate mucosal immunity, the immunoconjugates may be delivered intranasally. Adjuvants that are useful for stimulating mucosal immunity include proteosomes and Protollin™ (see, e.g., U.S. Pat. Nos. 6,803,402; 6,476,201; 5,985,284; 5,961,970; 5,726,292; 5,716,637; U.S. Patent Application Publication No. 2003/0044425). For a review of parenteral and mucosal delivery systems including delivery of antigens combined with an adjuvant see Kersten et al., Expert Rev. Vaccines (3:453-62 (2004).

A neurodegenerative retinal disease or disorder for which the compounds and methods described herein may be used for treating, curing, preventing, ameliorating the symptoms of, or slowing, inhibiting, or stopping the progression of, is a disease or disorder that leads to or is characterized by retinal neuronal cell loss, which is the cause of visual impairment. Such a disease or disorder includes but is not limited to macular degeneration (particularly dry form of macular degeneration), glaucoma, diabetic retinopathy, retinal detachment, retinal blood vessel (artery or vein) occlusion, retinitis pigmentosa, an inflammatory retinal disease, optical neuropathy, diabetic maculopathy, hemorrhagic retinopathy, retinopathy of prematurity, optic neuropathy, proliferative vitreoretinopathy, retinal dystrophy, ischemia-reperfusion related retinal injury, hereditary optic neuropathy, metabolic optic neuropathy, Stargardt's macular dystrophy, Sorsby's fundus dystrophy, Best disease, uveitis, a retinal injury, ischemia-reperfusion injury (such as that caused by transplant, surgical trauma, hypotension, thrombosis or trauma injury), traumatic injury to the optic nerve (such as by physical injury, excessive light exposure, or laser light), a retinal disorder associated with viral infection (e.g., cytomegalovirus or herpes simplex virus), a retinal disorder related to light overexposure, and retinal disorders associated with other neurodegenerative diseases such as Alzheimer's disease, multiple sclerosis, Parkinson's disease or other neurodegenerative diseases that affect brain cells, or other conditions such as AIDS. In another specific embodiment, the disease or disorder results from mechanical injury, chemical or drug-induced injury, thermal injury, radiation injury, light injury, laser injury. These methods are also useful for preventing ophthalmic injury from environmental factors such as light-induced oxidative retinal damage, laser-induced retinal damage, etc.

In another embodiment, a subject may be treated for diabetic retinopathy or diabetic maculopathy. Diabetes increases the permeability of blood vessel walls beneath the retina, allowing fluids and fatty exudates to accumulate in the macula. This accumulation causes macular edema, destabilizes RPE membranes, and causes abnormal blood vessel function, leading to light-exacerbated vision loss. Preventing the accumulation of these exudates (or phototoxic constituents, such as A2E) could protect the diabetic retina from degeneration.

An anti-A2E antibody or antigen-binding fragment thereof that inhibits degeneration of retinal cells or enhances survival of retinal cells such as retinal neuronal cells, particularly photoreceptor cells, may be particularly useful for treating retinal diseases such as the dry form of macular degeneration. The antibody or antigen-binding fragment thereof may be delivered systemically, such as intravenously, intramuscularly, intraperitoneally, or subdermally, or the antibody or fragment thereof may be delivered topically to the eye or intravitreally. Macugen® (pegaptanib sodium injection) is a pegylated single-stranded nucleic acid that specifically inhibits VEGF, which was very recently approved by the FDA for the treatment of neovascular (wet) ARMD. The nucleic acid is administered to patients by intravitreal injection every six weeks. Photoreceptor neurodegeneration is a sequela of retinal diseases, including but not limited to the dry form of macular degeneration. As described herein, dry or atrophic macular degeneration results in the loss of RPE cells and photoreceptors and is characterized by diminished retinal function due to an overall atrophy of the cells.

Macular degeneration as described herein is a disorder that affects the macula (central region of the retina) and results in the decline and loss of central vision. Age-related macular degeneration occurs typically in individuals over the age of 55 years. The etiology of age-related macular degeneration may include both an environmental influence and a genetic component (see, e.g, Iyengar et al., Am. J. Hum. Genet. 74:20-39 (2004) (Epub 2003 Dec. 19); Kenealy et al., Mol. Vis. 10:57-61 (2004); Gorin et al., Mol. Vis. 5:29 (1999)). More rarely, macular degeneration occurs in younger individuals, including children and infants, and generally the disorder results from a genetic mutation. Types of juvenile macular degeneration include Stargardt's disease (see, e.g., Glazer et al., Ophthalmol. Clin. North Am. 15:93-100, viii (2002); Weng et al., Cell 98:13-23 (1999)); Best's vitelliform macular dystrophy (see, e.g., Kramer et al., Hum. Mutat. 22:418 (2003); Sun et al., Proc. Natl. Acad. Sci. USA 99:4008-13 (2002)), Doyne's honeycomb retinal dystrophy (see, e.g., Kermani et al., Hum. Genet. 104:77-82 (1999)); Sorsby's fundus dystrophy, Malattia Levintinese, fundus flavimaculatus, and autosomal dominant hemorrhagic macular dystrophy (see also Seddon et al., Ophthalmology 108:2060-67 (2001); Yates et al., J. Med. Genet. 37:83-7 (2000); Jaakson et al., Hum. Mutat. 22:395-403 (2003)).

As used herein, a patient (or subject) may be any mammal, including a human, that may have or be afflicted with a neurodegenerative disease or condition, including an ophthalmic disease or disorder, or that may be free of detectable disease. Accordingly, the treatment may be administered to a subject who has an existing disease, or the treatment may be prophylactic, administered to a subject who is at risk for developing the disease or condition. A pharmaceutical composition may be a sterile aqueous or non-aqueous solution, suspension or emulsion, which additionally comprises a physiologically acceptable excipient (pharmaceutically acceptable or suitable excipient or carrier) (i.e., a non-toxic material that does not interfere with the activity of the active ingredient). Such compositions may be in the form of a solid, liquid, or gas (aerosol). Alternatively, compositions described herein may be formulated as a lyophilizate, or compounds may be encapsulated within liposomes using technology known in the art. Pharmaceutical compositions may also contain other components, which may be biologically active or inactive. Such components include, but are not limited to, buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, stabilizers, dyes, flavoring agents, and suspending agents and/or preservatives.

Any suitable excipient known to those of ordinary skill in the art may be employed in the pharmaceutical compositions described herein. Excipients for therapeutic use are well known, and are described, for example, in Remingtons Pharmaceutical Sciences, Mack Publishing Co. (A. R. Gennaro ed. 1985). In general, the type of excipient is selected based on the mode of administration. Pharmaceutical compositions may be formulated for any appropriate manner of administration, including, for example, intraocular, subconjunctival, topical, oral, nasal, intrathecal, rectal, vaginal, sublingual or parenteral administration, including subcutaneous, intravenous, intramuscular, intrasternal, intracavernous, intrameatal or intraurethral injection or infusion. For parenteral administration, the carrier preferably comprises water, saline, alcohol, a fat, a wax or a buffer. For oral administration, any of the above excipients or a solid excipient or carrier, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose and/or magnesium carbonate, may be employed.

A pharmaceutical composition (e.g., for oral administration or delivery by injection or for application as an eye drop) may be in the form of a liquid. A liquid pharmaceutical composition may include, for example, one or more of the following: sterile diluents such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition or a composition that is delivered ocularly is preferably sterile.

The anti-A2E antibodies and antigen-binding fragments thereof or the A2E conjugates described herein may be formulated for sustained or slow release. Such compositions may generally be prepared using well known technology and administered by, for example, oral, ocular, rectal or subcutaneous implantation, or by implantation at the desired target site. Sustained-release formulations may contain an agent dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Systemic drug absorption of a drug or composition administered via an ocular route is known to those skilled in the art (see, e.g., Lee et al., Int. J. Pharm. 233:1-18 (2002)). In one embodiment, an A2E specific antibody or antigen-binding fragment thereof or A2E conjugate is delivered by a topical ocular delivery method (see, e.g., Curr. Drug Metab. 4:213-22 (2003)). The composition may be in the form of an eye drop, salve, or ointment or the like, such as, aqueous eye drops, aqueous ophthalmic suspensions, non-aqueous eye drops, and non-aqueous ophthalmic suspensions, gels, ophthalmic ointments, etc. For preparing a gel, for example, carboxyvinyl polymer, methyl cellulose, sodium alginate, hydroxypropyl cellulose, ethylene maleic anhydride polymer and the like can be used. The dose of the composition of the present invention may differ, depending upon the patient's (e.g., human) condition, that is, stage of the disease, general health status, age, and other factors that a person skilled in the medical art will use to determine dose. When the composition of the present invention is used as eye drops, for example, one to several drops per unit dose, preferably 1 or 2 drops (about 50 μl per 1 drop), may be applied about 1 to about 6 times daily.

Pharmaceutical compositions may be administered in a manner appropriate to the disease to be treated (or prevented) as determined by persons skilled in the medical arts. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with neurodegeneration of retinal neuronal cells. Optimal doses may generally be determined using experimental models and/or clinical trials. The optimal dose may depend upon the body mass, weight, or blood volume of the patient. The dose depending upon any one of the aforementioned parameters may vary from 1 ng/ml to 10 mg/ml.

The following Examples are offered by way of illustration and not by way of limitation.

### EXAMPLES

### Example 1

### Preparation of Compound 39

This Example describes synthesis of a thiol-containing A2E compound according to the following reaction scheme (Reaction Scheme 8).

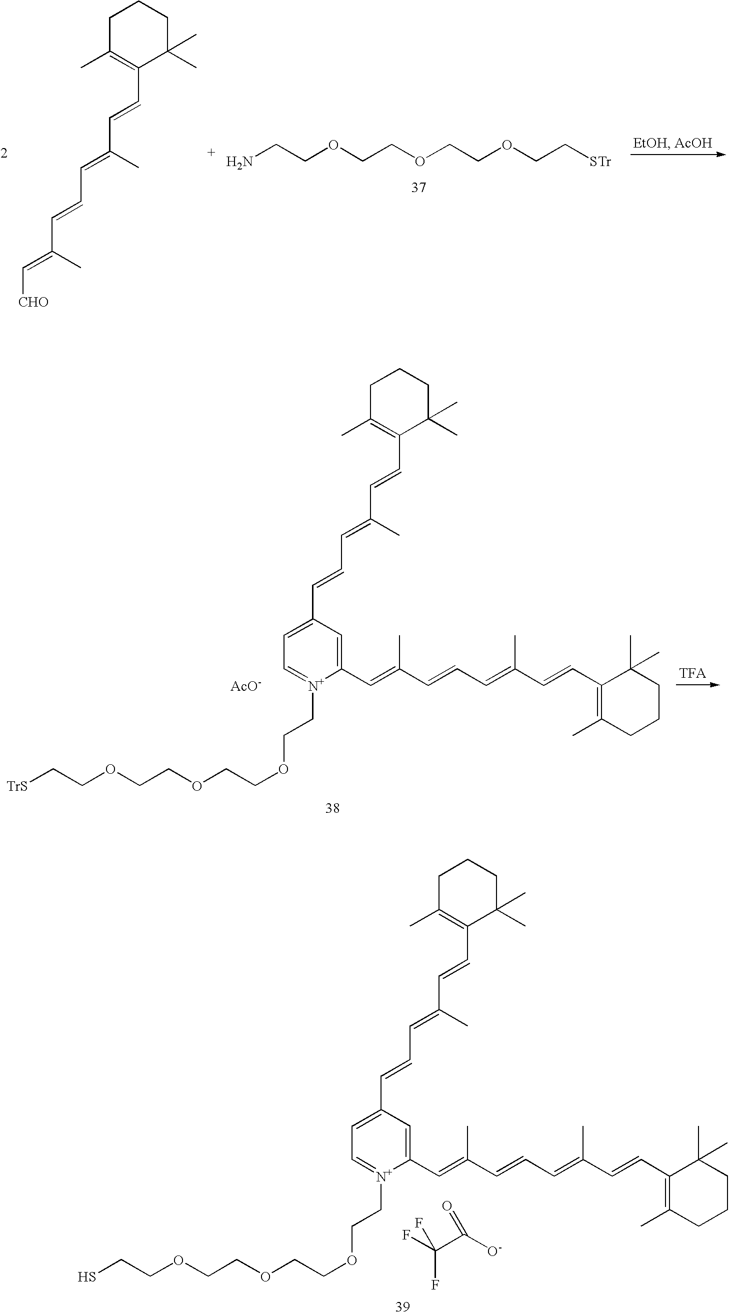

All-trans retinal (500 mg, 1.75 mmol) is dissolved in anhydrous ethanol (10 ml) and to this solution, 0.88 mmol of the amine (structure (37)) and 0.054 ml of acetic acid are added. The reaction vessel is wrapped in foil, and the mixture is stirred in the dark for 3 days at room temperature. The product is deprotected by treatment with 2% trifluoroacetic acid (TFA) in dichloromethane at room temperature for 5 hr and purified by flash chromatography on silica gel using 1:9 mixture of methanol and dichloromethane containing 0.1% of TFA to elute the product.

### Example 2

### Preparation of a Compound 8

This Example describes the synthesis of a compound 8 as described herein (see Reaction Scheme 3).

A compound 6 is prepared according to the procedure described in Ren et al. (J. Am. Chem. Soc. 119:3619-20 (1997)). Compound 6 is oxidized with manganese dioxide similarly as described in Samokyszyn et al. (J. Biol. Chem. 275:6908-14 (2000)). Compound 6 (55 mg, 0.1 mmol) is stirred with 1 g of manganese dioxide in 10 mL of dichloromethane at room temperature in the dark for 90 hr. The MnO₂ is removed by filtration, and the solvent is removed under vacuum. The product is purified by flash chromatography on silica gel eluting with 20:1 hexanes:ethyl acetate to yield 28 mg of a compound 8 (50%) as a mixture of isomers.

### Example 3

### Preparation of Compound 9

This Example describes the synthesis of a compound 9 as described herein (see Reaction Scheme 3).

Synthesis of this compound is performed in a similar manner to the method in Ren et al., supra. A mixture of compound 8 (see Example 2) (28 mg, 0.05 mmol) and iodoethanol (4.4 μL, 0.055 mmol) in nitromethane (3 mL) is heated under reflux in the dark overnight (12 hr). The crude product is purified on silica gel column in a dark room (or otherwise protected from light) to yield compound 9 (18 mg, 50%) in the form of a bright red fluorescent syrup.

### Example 4

### Preparation of Compound 11

This Example describes the synthesis of compound 11 as described herein (see Reaction Scheme 4).

Compound 10 is prepared as described (Tanaka et al., J. Org. Chem. 66:3099-3110 (2001)). Then, to a solution of compound 10 (160 mg, 0.55 mmol) in dichloromethane (4 mL), is added manganese dioxide (200 mg) at room temperature, and the mixture is stirred for 15 min. The reaction mixture is filtered and concentrated in vacuo to give the crude product, which is purified by flash chromatography on silica gel eluting with 7:3 hexanes:ethyl acetate to yield compound 11 (150 mg, 94%).

### Example 5

### Preparation of Compound 13

This Example describes the synthesis of a compound 13 as described herein (see Reaction Scheme 4).

Triphenylphosphonium reagent (12) (see Reaction Scheme 4) is prepared as described (Curley et al., J. Org. Chem. 49:1941-1944 (1984)). To a solution of reagent 12 (0.31 g, 0.6 mmol) in anhydrous ethanol (10 mL), a 21% solution of sodium ethoxide in ethanol (0.22 mL, 0.6 mmol) is added dropwise at 0° C. The resulting dark-red solution is stirred for 0.5 hr followed by dropwise addition of a solution of the compound of structure (11) (150 mg, 0.51 mmol) in anhydrous ethanol (5 mL) at 0° C. The reaction mixture is continuously stirred in the dark for 1 hr. The product is purified by flash chromatography on silica gel eluting with 10:1 hexanes:ethyl acetate to yield compound 13 (170 mg, 70%).

### Example 6

### Preparation of Compound 14

This Example describes the synthesis of a compound 14 as described herein (see Reaction Scheme 4).

To a THF solution (5 mL) of the compound 13 (130 mg, 0.28 mmol) is added tetrabutylammonium fluoride (TBAF) (86 mg, 0.33 mmol) at room temperature. After being stirred at room temperature for 75 min, the reaction mixture is concentrated in vacuo to provide the crude product. The product is purified by flash chromatography on silica gel eluting with 9:1 methanol:dichloromethane to yield compound 13 (100 mg, 100%). To a solution of this compound (13) (100 mg, 0.28 mmol) in dichloromethane (4 mL), is added manganese dioxide (300 mg) at room temperature, and the mixture is stirred for 15 min. The reaction mixture is filtered and concentrated in vacuo to give the crude product, which is purified by flash chromatography on silica gel using 7:3 hexanes:ethyl acetate to yield compound 14 (95 mg, 94%).

### Example 7

### Preparation of Compound 16

This Example describes the synthesis of a compound 16 as described herein (see Reaction Scheme 4).

Compound 15 (see Reaction Scheme 4) is prepared according to procedures practiced in the art. To a solution of compound of structure 15 (0.43 g, 0.6 mmol) in anhydrous ethanol (10 mL) is added dropwise a 21% solution of sodium ethoxide in ethanol (0.22 mL, 0.6 mmol) at 0° C. The resulting dark-red solution is stirred for 0.5 hr followed by dropwise addition of a solution of compound 14 (184 mg, 0.51 mmol) in anhydrous ethanol (5 mL) at 0° C. The reaction mixture is continuously stirred in the dark for 1 hr. The product is purified by flash chromatography on silica gel eluting with 10:1 hexanes:ethyl acetate to yield compound 16 (0.25 g, 70%).

### Example 8

### Preparation of Compound 17

This Example describes the synthesis of a compound 17 as described herein (see Reaction Scheme 4).

A mixture of the compound 16 prepared as described in Example 7 (57 mg, 0.08 mmol) and iodoethanol (8.0 μL, 0.1 mmol) in nitromethane (3 mL) is heated under reflux in the dark overnight (12 hr). The crude product is purified on silica gel column (in a dark room) to give 35 mg of a bright red fluorescent syrup. This compound is treated with trifluoroacetic acid in dichloromethane at room temperature to afford the yield the bis trifluoroacetate compound 17 (35 mg, 50%).

### Example 9

### Preparation of Compound 21

This Example describes the synthesis of a compound 21 as described herein (see Reaction Scheme 5).

All-trans-retinal is converted to its diethyl acetate according to methods practiced in the art (see, e.g., Patent FR1313917; Chem. Abstr. 59 10135f(1963)), which is brominated with N-bromosuccinimide in carbon tetrachloride similar to the method described in Sheves et al. (J. Am. Chem. Soc. 106: 2435-2437 (1984)) to yield compound 19. The compound 19 is converted to a compound 21 as follows. To a solution of sodium ethoxide in ethanol (1 mmol in 10 mL), N-Boc aminoethane thiol (compound of structure (20)) is added (0.18 g, 1 mmol). After stirring at room temperature for 10 min, the mixture is added to a solution of compound 19 (0.44 g, 1 mmol) in ethanol (10 mL). The resulting mixture is stirred at 25° C. for 30 min, diluted with ethyl acetate (100 mL) and water (50 mL), and then the organic layer is separated. The organic layer is washed with water, dried over sodium sulfate, and then evaporated in vacuo. The product (0.50 g, 94%) is used without further purification in the next step (see Example 10).

### Example 10

### Preparation of Compound 22

This Example describes the synthesis of a compound 22 as described herein (see Reaction Scheme 5).

Compound 21 is deprotected to provide the aldehyde compound 22 by stirring a solution of compound 21 in THF with aqueous oxalic acid at room temperature. The reaction mixture is monitored by HPLC or TLC to avoid deprotection of the Boc group. The product is purified by flash chromatography on silica gel using a mixture of hexanes and ethyl acetate (1:1) to yield a compound 22 (0.32 g, 70%).

### Example 11

### Preparation of a Compound 25

This Example describes the synthesis of a compound 25 as described herein (see Reaction Scheme 5).

Compound 22 (160 mg, 0.35 mmol) (see Example 10) is dissolved in anhydrous ethanol (3 mL) and to this solution, 0.35 mmol of ethanolamine and 0.35 mmol of acetic acid are added. The reaction vessel is wrapped in foil, and the mixture is stirred in the dark for 5 hr at room temperature. To the mixture, a solution of all-trans-retinal (0.35 mmol) in ethanol (2 mL) is added, and the reaction mixture is stirred in the dark for 2 days at room temperature to yield compound 24 as a mixture of isomers. The products are deprotected with TFA and separated using high performance liquid chromatography on C-18 reverse phase column using a gradient of acetonitrile in 0.1% trifluoroacetic acid.

### Example 12

### Preparation of Compound 28

This Example describes the synthesis of a compound 28 as described herein (see Reaction Scheme 6).

The 2-hydroxypyridine 4-aldehyde (compound 26) is obtained according to a method described in Ren et al. (J. Am. Chem. Soc. 119:3619-20 (1997)). This compound is protected with tert-butyldimethylsilyl chloride to yield a compound 27 (see, e.g., Tanaka et al., J. Org. Chem. 66: 3099-3110 (2001)). A 21% solution of sodium ethoxide in ethanol (0.22 mL, 0.6 mmol) is added dropwise at 0° C. to a solution of a compound 12 (see Reaction Scheme 4) (0.31 g, 0.6 mmol) in anhydrous ethanol (10 mL). The resulting dark-red solution is stirred for 0.5 hr followed by dropwise addition of a solution of a compound 27 (121 mg, 0.51 mmol) in anhydrous ethanol (5 mL) at 0° C. The reaction mixture is continuously stirred in the dark for 1 hr. The product is purified by flash chromatography on silica gel using 10:1 hexanes:ethyl acetate for elution to yield compound 28 (151 mg, 70%).

### Example 13

### Preparation of Compound 29

This Example describes the synthesis of a compound 29 as described herein (see Reaction Scheme 6).

To a THF solution of compound 28 (118 mg, 0.28 mmol) (5 mL) is added tetrabutylammonium fluoride (TBAF) (86 mg, 0.33 mmol) at room temperature. After being stirred at room temperature for 75 min, the reaction mixture is concentrated in vacuo to give the crude product. The product is purified by flash chromatography on silica gel, eluting with 9:1 methanol:dichloromethane to provide the corresponding alcohol (86 mg, 100%). To a mixture of this alcohol and pyridine (70 μL) in dichloromethane (2 mL), is added triflic anhydride (0.31 mmol) at 0° C. The reaction mixture is stirred at room temperature for 4 hr and quenched by addition of saturated aqueous solution of ammonium chloride (2 mL). The mixture is extracted with ether (3 times, 5 mL each), and the combined organic layers are washed with brine (5 mL) and dried over anhydrous magnesium sulfate. The crude product is purified by flash chromatography on silica gel to yield compound 29 (99 mg, 80%).

### Example 14

### Preparation of Compound 31

This Example describes the synthesis of a compound 31 as described herein (see Reaction Scheme 6).

A mixture of a compound 29 (88 mg, 0.2 mmol); tin reagent (compound 30) (86 mg, 0.22 mmol); LiCl (10 mg, 0.24 mmol); Pd(PPh₃)₄ (12 mg, 5 mol %); and CuI (19 mg, 5 mol %) in dioxane is heated at reflux under argon overnight. The reation mixture is filtered to remove insoluble material, and the filtrate is concentrated in vacuo. The crude product is purified on silica gel to yield compound 31 (69 mg, 88%).

### Example 15

### Preparation of Compound 32

This Example describes the synthesis of a compound 32 as described herein (see Reaction Scheme 6).

Compound 31 (69 mg, 0.17 mmol) (see Example 14) is dissolved in ethanol (2 ml), and to this solution is added 1 mL of 0.5M solution of lithium hydroxide. The reaction mixture is stirred at 40° C. for 3 hr, and ethanol is evaporated under vacuum. To the residue, 1 ml of 0.5 M HCl is added, and the mixture is extracted with ethyl acetate (3 times×5 mL each). The combined extracts are washed successively with saturated aqueous sodium bicarbonate solution, brine, and water and dried over magnesium sulfate. The solution is evaporated to yield a crude acid, which is dried under vacuum, dissolved in anhydrous THF, treated with DCC (0.17 mmol) and mono-Boc protected ethanolamine (27 mg, 0.17 mmol). After stirring for 12 hr, the mixture is filtered to remove precipitated dicyclohexylurea and concentrated in vacuo. The crude product is purified on silica gel to yield compound 32 (43 mg, 50%).

### Example 16

### Preparation of Compound 33

This Example describes the synthesis of a compound 33 as described herein (see Reaction Scheme 6).

A mixture of the compound 32 (25 mg, 0.05 mmol) and iodoethanol (4.4 μL, 0.055 mmol) in nitromethane (3 mL) is heated under reflux in the dark overnight (12 hr). The crude product is deprotected by treatment with trifluoroacetic acid in methylene chloride and purified by reverse phase HPLC using a gradient of acetonitrile in 0.1% trifluoroacetic acid to yield compound 33 (20 mg, 50%) as a bright red fluorescent syrup.

### Example 17

### Preparation of Compound 39 Conjugated to KLH

This Example describes the conjugation of a compound 39 as described herein (see Example 1).

The compound 39 is conjugated to Imject® maleimide-activated mariculture keyhole limpet hemocyanin (mcKLH, Pierce Biotechnolgoy) according to the manufacturer protocols. All procedures are performed under dim red light to minimize decomposition of A2E derivatives. The number of molecules of A2E derivative bound per molecule of KLH (i.e., the extent of conjugation) is quantified by measuring absorbance of a solution of the conjugate at 430 nm and calculating the concentration of A2E (ε=30,000).

### Example 18

### Preparation of Compound 9 Conjugated to BSA

This Example describes the conjugation of a compound 9 as described herein (see Example 3).

Compound 9 is conjugated to BSA according to a method described in Pestka et al., J. Food Prot. 48:953-57 (1985)).

### Example 19

### Preparation of Compound 17 Conjugated to BSA

This Example describes the conjugation of a compound 17 as described herein (see Reaction Scheme 4; Example 8).

Compound 17 is coupled to BSA using standard glutaraldehyde-mediated coupling procedures. All procedures are performed under dim red light to minimize decomposition of A2E derivatives. The number of molecules of A2E derivative that are conjugated per molecule of BSA is quantified by measuring absorbance of a solution of the conjugate at 430 nm and calculating concentration of A2E (ε=30,000).

### Example 20

### Preparation of Compound 41

This Example describes synthesis of a carboxyl-containing A2E compound according to the following reaction scheme (Reaction Scheme 9).

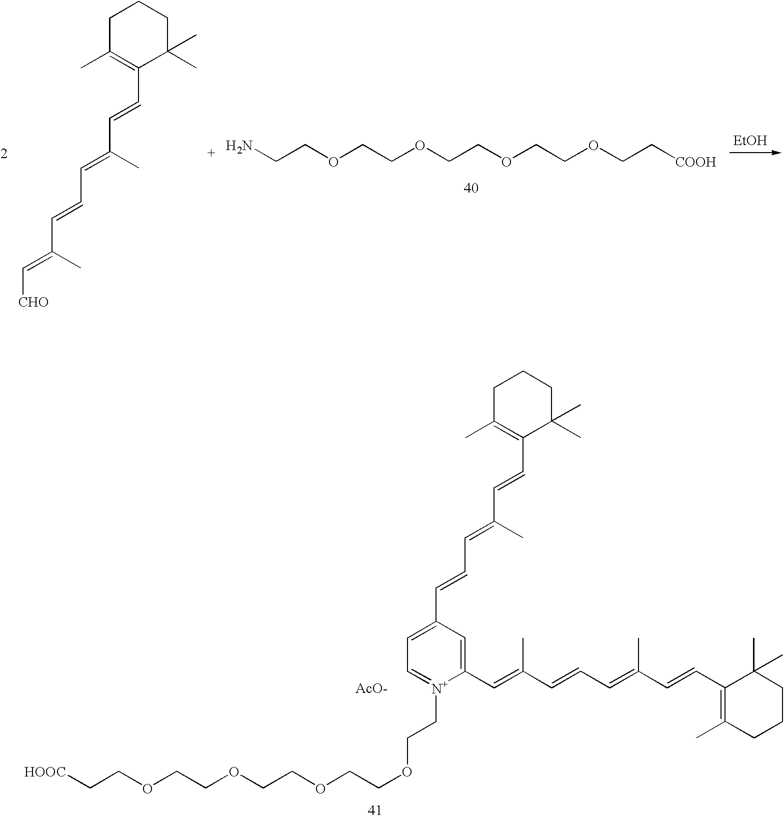

All-trans retinal (284 mg, 1 mmol) is dissolved in anhydrous ethanol (5 ml) and to this solution, 0.5 mmol of the amine (structure (40), available from Quanta BioDesign) is added. The reaction vessel is wrapped in foil, and the mixture is stirred in the dark for 5 days at room temperature. After removal of the solvent and purification by flash chromatography on silica gel using 15:85 mixture of methanol and dichloromethane containing 2% of acetic acid to elute the product, 28 mg (6.5%) of compound 41 is obtained as dark red oil.

### Example 21

### Preparation of Compound 44

This Example describes synthesis of an amino-containing A2E compound according to the following reaction scheme (Reaction Scheme 10).

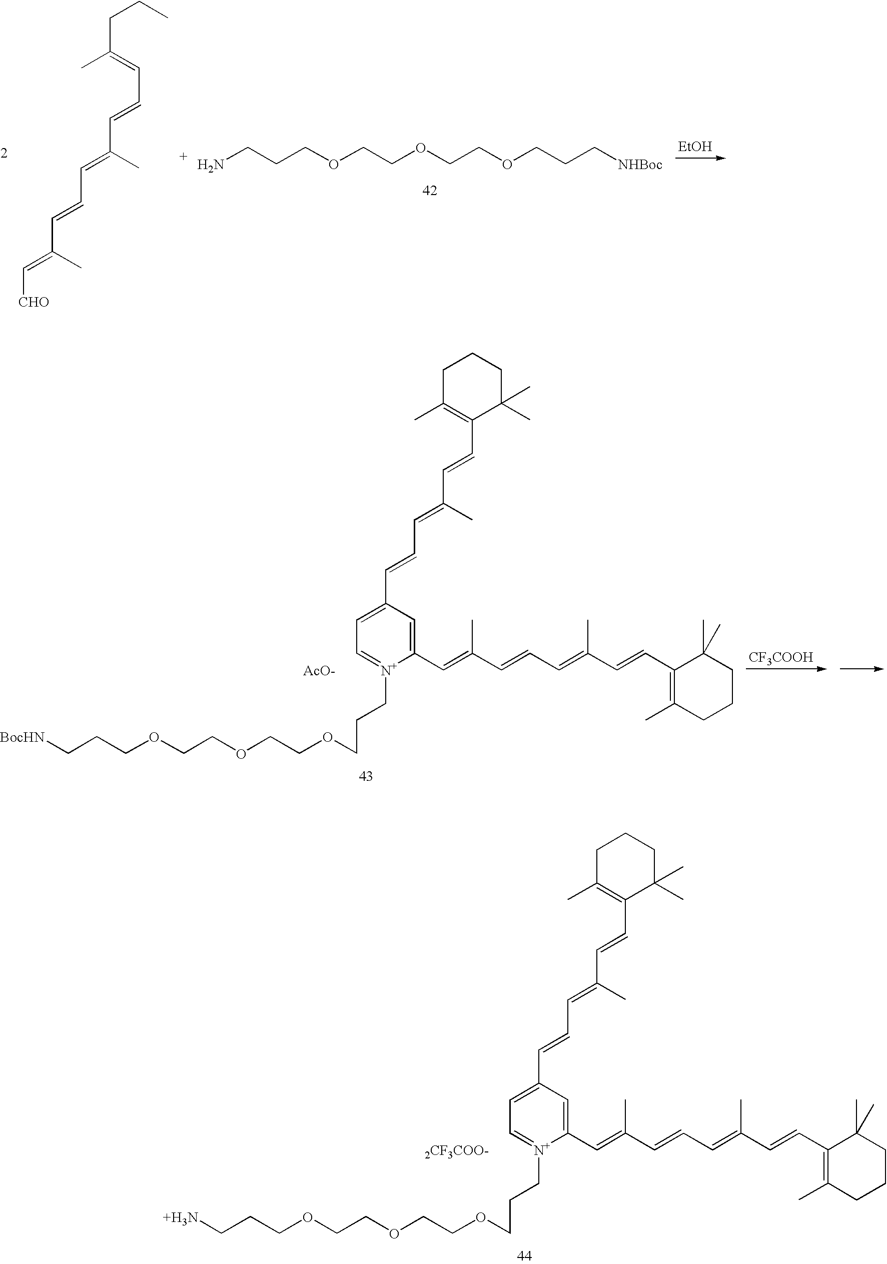

All-trans retinal (580 mg, 2 mmol) is dissolved in anhydrous ethanol (5 ml) and to this solution, 160 mg (0.5 mmol) of the amine (structure (42), available from Quanta BioDesign) is added, followed by 30 mg (0.5 mmol) of acetic acid. The reaction vessel is wrapped in foil, and the mixture is stirred in the dark for 7 days at room temperature. After removal of the solvent and purification by flash chromatography on silica gel using 15:85 mixture of methanol and dichloromethane containing 2% of acetic acid to elute the product, 75 mg (16.5%) of compound 44 is obtained as dark red oil. The compound is deprotected by treatment with trifluoroacetic acid in dichloromethane to yield compound 44.

### Example 22

### Immunogenicity of A2E Immunoconjugates

This Example describes methods for testing, evaluating, and characterizing the immunogenicity of an A2E immunoconjugates. An A2E immunoconjugates as described herein may be prepared according to the exemplary procedures described in the aforementioned Examples.

An A2E immunoconjugate (e.g., an A2E like compound described herein conjugated to a carrier polypeptide such as KLH) is mixed with an adjuvant (e.g., Ribi Adjuvant System). Groups of 2 rabbits are injected with different A2E immunoconjugates according to immunization protocols practiced in the art. Rabbits receive at least one booster immunizations 2-4 weeks after the initial immunization. Sera is prepared from blood obtained from the animals and tested in an immunoassay, such as an Ochterlony assay or an ELISA to determine the titer of the sera against the A2E immunoconjugates used as immunogens. Booster immunizations are repeated as needed until a maximum antibody titer is reached. The sera may be used for testing of the antibodies in the cell culture assay described in the following Example 24.

An A2E immunoconjugate that provokes an immune response in rabbits is then used as an immunogen in mice. Groups of 5-10 mice per A2E immunoconjugates are immunized with the conjugate mixed with adjuvant according to standard immunization procedures used for immunizing mice. Different groups of mice may be immunized with different amounts of conjugate to determine an amount of the A2E conjugate that stimulates an immune response. The immune response of the mice is monitored by an immunoassay, such as ELISA. The sera may be used for testing of the antibodies in the cell culture assay described in Example 24.

Mice with the highest titer of anti-A2E antibodies can be boosted and the spleens obtained for isolation of monoclonal antibodies. Mice are boosted with the A2E conjugate 3-5 days prior to spleen removal. Standard procedures for hybridizing spleen B cells with an immortalized cell fusion partner and isolating specific antibodies are followed. The capability of the A2E monoclonal antibodies to inhibit degeneration of a retinal cell (or enhance or prolong survival of a retinal cell, such as a retinal neuronal cell) is examined in the retinal cell culture assay described in Example 24.

### Example 23

### Preparation of Retinal Neuronal Cell Culture System

This Example describes methods for preparing a long-term culture of retinal neuronal cells.

All compounds and reagents were obtained from Sigma Aldrich Chemical Corporation (St. Louis, Mo.) except as noted.

Retinal Neuronal Cell Culture

Porcine eyes are obtained from Kapowsin Meats, Inc. (Graham, Wash.). Eyes are enucleated, and muscle and tissue are cleaned away from the orbit. Eyes are cut in half along their equator and the neural retina is dissected from the anterior part of the eye in buffered saline solution, according to standard methods known in the art. Briefly, the retina, ciliary body, and vitreous are dissected away from the anterior half of the eye in one piece, and the retina is gently detached from the clear vitreous. Each retina is dissociated with papain (Worthington Biochemical Corporation, Lakewood, N.J.), followed by inactivation with fetal bovine serum (FBS) and addition of 134 Kunitz units/ml of DNaseI. The enzymatically dissociated cells are triturated and collected by centrifugation, resuspended in Dulbecco's modified Eagle's medium (DMEM)/F12 medium (Gibco BRL, Invitrogen Life Technologies, Carlsbad, Calif.) containing 25 μg/ml of insulin, 100 μg/ml of transferrin, 60 μM putrescine, 30 nM selenium, 20 nM progesterone, 100 U/ml of penicillin, 100 μg/ml of streptomycin, 0.05 M Hepes, and 10% FBS. Dissociated primary retinal cells are plated onto Poly-D-lysine- and Matrigel- (BD, Franklin Lakes, N.J.) coated glass coverslips that are placed in 24-well tissue culture plates (Falcon Tissue Culture Plates, Fisher Scientific, Pittsburgh, Pa.). Cells are maintained in culture for 5 days to one month in 0.5 ml of media (as above, except with only 1% FBS) at 37° C. and 5% CO₂.

Immunocytochemistry Analysis

The retinal neuronal cells are cultured for 1, 3, 6, and 8 weeks, and the cells are analyzed by immunohistochemistry at each time point. Immunocytochemistry analysis is performed according to standard techniques known in the art. Rod photoreceptors are identified by labeling with a rhodopsin-specific antibody (mouse monoclonal, diluted 1:500; Chemicon, Temecula, Calif.). An antibody to mid-weight neurofilament (NFM rabbit polyclonal, diluted 1:10,000, Chemicon) is used to identify ganglion cells; an antibody to β3-tubulin (G7121 mouse monoclonal, diluted 1:1000, Promega, Madison, Wis.) is used to generally identify interneurons and ganglion cells, and antibodies to calbindin (AB1778 rabbit polyclonal, diluted 1:250, Chemicon) and calretinin (AB5054 rabbit polyclonal, diluted 1:5000, Chemicon) are used to identify subpopulations of calbindin- and calretinin-expressing interneurons in the inner nuclear layer. Briefly, the retinal cell cultures are fixed with 4% paraformaldehyde (Polysciences, Inc, Warrington, Pa.) and/or ethanol, rinsed in Dulbecco's phosphate buffered saline (DPBS), and incubated with primary antibody for 1 hour at 37° C. The cells are then rinsed with DPBS, incubated with a secondary antibody (Alexa 488- or Alexa 568-conjugated secondary antibodies (Molecular Probes, Eugene, Oreg.)), and rinsed with DPBS. Nuclei are stained with 4′,6-diamidino-2-phenylindole (DAPI, Molecular Probes), and the cultures are rinsed with DPBS before removing the glass coverslips and mounting them with Fluoromount-G (Southern Biotech, Birmingham, Ala.) on glass slides for viewing and analysis.

Survival of mature retinal neurons after varying times in culture is indicated by the histochemical analyses. Photoreceptor cells are identified using a rhodopsin antibody; ganglion cells are identified using an NFM antibody; and amacrine and horizontal cells are identified by staining with an antibody specific for calretinin.

Cultures are analyzed by counting rhodopsin-labeled photoreceptors and NFM-labeled ganglion cells using an Olympus 1X81 or CZX41 microscope (Olympus, Tokyo, Japan). Twenty fields of view are counted per coverslip with a 20× objective lens. Six coverslips are analyzed by this method for each condition in each experiment. Cells that are not exposed to any stressor are counted, and cells exposed to a stressor are normalized to the number of cells in the control.

### Example 24

### Effect of an Anti-A2E Antibody on Retinal Cell Survival

This Example describes the use of the mature retinal cell culture system that comprises a cell stressor for determining the effects of an antibody that specifically binds to A2E on the viability of the retinal cells.

Retinal cell cultures are prepared as described in Example 2. A2E is added as a retinal cell stressor. After culturing the cells for 1 week, a chemical stress, A2E, is applied. A2E is diluted in ethanol and added to the retinal cell cultures at concentration of 0, 10 μM, 20 μM, and 40 μM. Cultures are treated for 24 and 48 hours. A2E is obtained from Dr. Koji Nakanishi (Columbia University, New York City, N.Y.) or is synthesized according to the method of Parish et al. (Proc. Natl. Acad. Sci. USA 95:14602-13 (1998)). An antibody that specifically binds to A2E is then added to the culture. To other retinal cell cultures, an antibody that specifically binds to A2E is added before application of the stressor or is added at the same time that A2E is added to the retinal cell culture. The cultures are maintained in tissue culture incubators for the duration of the stress at 37° C. and 5% CO₂. The cells are then analyzed by immunocytochemistry as described in Example 1.

Apoptosis Analysis

Retinal cell cultures are prepared as described in Example 20 and cultured for 2 weeks and then exposed to white light stress at 6000 lux for 24 hours followed by a 13-hour rest period. A device was built to uniformly deliver light of specified wavelengths to specified wells of the 24-well plates. The device contained a fluorescent cool white bulb (GE P/N FC12T9/CW) wired to an AC power supply. The bulb is mounted inside a standard tissue culture incubator. White light stress is applied by placing plates of cells directly underneath the fluorescent bulb. The CO₂ levels are maintained at 5%, and the temperature at the cell plate is maintained at 37° C. The temperature was monitored by using thin thermocouples. The light intensities for all devices were measured and adjusted using a light meter from Extech Instruments Corporation (P/N 401025; Waltham, Mass.). An antibody that specifically binds to A2E is added to wells of the culture plates prior to exposure of the cells to white light and is added to other wells of the cultures after exposure to white light. To assess apoptosis, TUNEL is performed as described herein.

Apoptosis analysis is also performed after exposing retinal cells to blue light. Retinal cell cultures are cultured as described in Example 20. After culturing the cells for 1 week, a blue light stress is applied. Blue light is delivered by a custom-built light-source, which consists of two arrays of 24 (4×6) blue light-emitting diodes (Sunbrite LED P/N SSP-01TWB7UWB12), designed such that each LED is registered to a single well of a 24 well disposable plate. The first array is placed on top of a 24 well plate full of cells, while the second one is placed underneath the plate of cells, allowing both arrays to provide a light stress to the plate of cells simultaneously. The entire apparatus is placed inside a standard tissue culture incubator. The CO₂ levels are maintained at 5%, and the temperature at the cell plate is maintained at 37° C. The temperature is monitored with thin thermocouples. Current to each LED is controlled individually by a separate potentiometer, allowing a uniform light output for all LEDs. Cell plates are exposed to 2000 lux of blue light stress for either 2 hours or 48 hours, followed by a 14 hour rest period. An antibody that specifically binds to A2E is added to wells of the culture plates prior to exposure of the cells to blue light and is added to other wells of the cultures after exposure to blue light. To assess apoptosis, TUNEL is performed as described herein.

To assess apoptosis, TUNEL is performed according to standard techniques practiced in the art and according to the manufacturer's instructions. Briefly, the retinal cell cultures are first fixed with 4% paraformaldehyde and then ethanol, and then rinsed in DPBS. The fixed cells are incubated with TdT enzyme (0.2 units/μl final concentration) in reaction buffer (Fermentas, Hanover, Md.) combined with Chroma-Tide Alexa568-5-dUTP (0.1 μM final concentration) (Molecular Probes) for 1 hour at 37° C. Cultures are rinsed with DPBS and incubated with primary antibody either overnight at 4° C. or for 1 hour at 37° C. The cells are then rinsed with DPBS, incubated with Alexa 488-conjugated secondary antibodies, and rinsed with DPBS. Nuclei are stained with DAPI, and the cultures are rinsed with DPBS before removing the glass coverslips and mounting them with Fluoromount-G on glass slides for viewing and analysis.

Cultures are analyzed by counting TUNEL-labeled nuclei using an Olympus 1X81 or CZX41 microscope (Olympus, Tokyo, Japan). Twenty fields of view are counted per coverslip with a 20× objective lens. Six coverslips are analyzed by this method for each condition. Cells that are not exposed to the anti-A2E antibody are counted, and cells exposed to the antibody are normalized to the number of cells in the control. Data are analyzed using the unpaired Student's t-test.

From the foregoing, it will be appreciated that, although specific embodiments of the invention have been described herein for purposes of illustration, various modifications may be made without deviating from the spirit and scope of the invention.

1. A compound having the following structure (1): 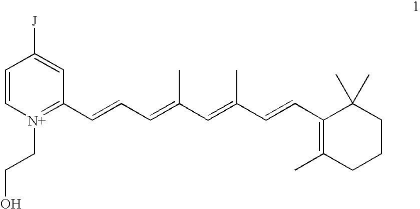
or a pharmaceutically acceptable acid addition salt thereof;
wherein
J is -Z¹-Y, -Z²-R⁰—Y, -Z²-R⁰-Z³-Y, or -Z²-Y′—R⁰,
Z¹ is a divalent C₁-C₄₀ alkyl,
Z² is a divalent C₁-C₄₀ alkyl,
Z³ is a polyethylene glycol having the formula —(CH₂CH₂O)ₙCH₂CH₂— wherein n=2-8, or —R⁴—CH₂—, wherein R⁴ is C₁-C₄₀ alkylene or C₁-C₄₀ heteroalkylene;
R⁰ is a monovalent or divalent optionally substituted homocycle, aryl, heteroaryl, or heterocycle; and
wherein Y is a monovalent electrophilic or nucleophilic moiety suitable for covalent attachment of the compound to an amino acid,
and wherein Y′ is a divalent electrophilic or nucleophilic moiety suitable for covalent attachment of the compound to an amino acid.

2. The compound according to claim 1 wherein J is -Z¹-Y.

3. The compound according to claim 1 wherein J is -Z²-R⁰—Y.

4. The compound according to claim 1 wherein J is -Z²-R⁰-Z³-Y.

5. The compound according to claim 4 wherein Z³ is a polyethylene glycol having the formula —(CH₂CH₂O)ₙCH₂CH₂— wherein n=2-8.

6. The compound according to claim 4 wherein Z³ is —R⁴—CH₂—, and wherein R⁴ is C₁-C₄₀ alkylene or C₁-C₄₀ heteroalkylene.

7. The compound according to claim 6 wherein R⁴ is C₁-C₄₀ alkylene.

8. The compound according to claim 6 wherein R⁴ is C₁-C₄₀ heteroalkylene.

9. The compound according to claim 8 wherein C₁-C₄₀ heteroalkylene comprises amide, disulfide, —O—, —S—, or sulfonamide.

10. The compound according to claim 6 wherein R⁴ is C₁-C₂₀ alkylene or C₁-C₂₀ heteroalkylene.

11. The compound according to claim 10 wherein R⁴ is C₁-C₂₀ alkylene.

12. The compound according to claim 10 wherein R⁴ is C₁-C₂₀ heteroalkylene.

13. The compound according to claim 12 wherein C₁-C₂₀ heteroalkylene comprises amide, disulfide, —O—, —S—, or sulfonamide.

14. The compound according to claim 1 wherein Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

15. The compound according to claim 1 wherein Y is —SH, —NH₂, or —C(═O)OH.

16. The compound according to claim 1 wherein J is -Z²-Y′—R⁰.

17. The compound according to claim 11 wherein Y′ is —O—.

18. A compound having the following structure (2): 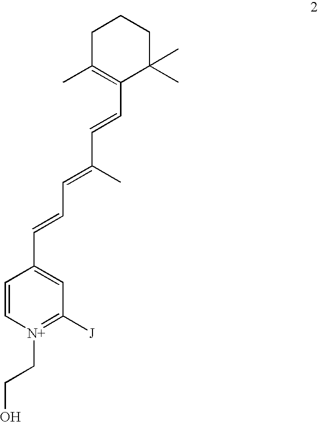
or a pharmaceutically acceptable acid addition salt thereof,
wherein
J is -Z¹-Y, -Z²-R⁰—Y, -Z²-R⁰-Z³-Y, or -Z²-Y′—R⁰,
Z¹ is a divalent C₁-C₄₀ alkyl,
Z² is a divalent C₁-C₄₀ alkyl,
Z³ is a polyethylene glycol having the formula —(CH₂CH₂O)ₙCH₂CH₂— wherein n=2-8, or —R⁴—CH₂—, wherein R⁴ is C₁-C₄₀ alkylene or C₁-C₄₀ heteroalkylene;
R⁰ is an monovalent or divalent optionally substituted homocycle, aryl, heteraryl, or heterocycle; and
wherein Y is a monovalent electrophilic or nucleophilic moiety suitable for covalent attachment of the compound to an amino acid,
and wherein Y′ is a divalent electrophilic or nucleophilic moiety suitable for covalent attachment of the compound to an amino acid.

19. The compound according to claim 18 wherein J is -Z¹-Y.

20. The compound according to claim 18 wherein J is -Z²-R⁰—Y.

21. The compound according to claim 18 wherein J is -Z²-R⁰-Z³-Y.

22. The compound according to claim 21 wherein Z³ is a polyethylene glycol having the formula —(CH₂CH₂O)ₙCH₂CH₂— wherein n=2-8.

23. The compound according to claim 21 wherein Z³ is —R⁴—CH₂—, and wherein R⁴ is C₁-C₄₀ alkylene or C₁-C₄₀ heteroalkylene.

24. The compound according to claim 23 wherein R⁴ is C₁-C₄₀ alkylene.

25. The compound according to claim 23 wherein R⁴ is C₁-C₄₀ heteroalkylene.

26. The compound according to claim 25 wherein C₁-C₄₀ heteroalkylene comprises amide, disulfide, —O—, —S—, or sulfonamide.

27. The compound according to claim 23 wherein R⁴ is C₁-C₂₀ alkylene or C₁-C₂₀ heteroalkylene.

28. The compound according to claim 27 wherein R⁴ is C₁-C₂₀ alkylene.

29. The compound according to claim 27 wherein R⁴ is C₁-C₂₀ heteroalkylene.

30. The compound according to claim 29 wherein C₁-C₂₀ heteroalkylene comprises amide, disulfide, —O—, —S—, or sulfonamide.

31. The compound according to claim 18 wherein Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

32. The compound according to claim 18 wherein Y is —SH, —NH₂, or —C(═O)OH.

33. The compound according to claim 18 wherein J is -Z²-Y′—R⁰.

34. The compound according to claim 33 wherein Y′ is —O—.

35. A compound having the following structure (3): 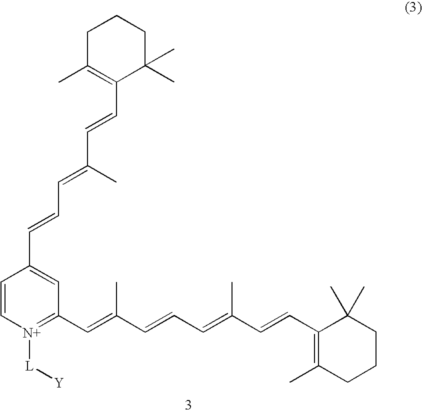
or a pharmaceutically acceptable acid addition salt thereof,
wherein L is a divalent linker group -R₁-, -R₂-, or -R₃-, and
R₁ is divalent C₁-C₆ alkyl;
R₂ is C₁-C₄₀ alkylene or C₁-C₄₀ heteroalkylene; and
R₃ is a polyethylene glycol having the formula —(CH₂CH₂O)ₙCH₂CH₂— wherein n=2-12;
and wherein Y is an electrophilic or nucleophilic moiety suitable for reaction of the compound with an amino acid.

36. The compound of claim 35 wherein L is -R₁-.

37. The compound of claim 35 wherein L is -R₂-.

38. The compound of claim 35 wherein L is -R₃-.

39. The compound of claim 37 wherein -R₂- is C₁-C₄₀ alkylene.

40. The compound of claim 37 wherein -R₂- is C₁-C₄₀ heteroalkylene.

41. The compound of claim 37 wherein -R₂- is C₁-C₂₀ alkylene or C₁-C₂₀ heteroalkylene.

42. The compound of claim 37 wherein -R₂- is C₁-C₂₀ alkylene.

43. The compound of claim 37 wherein -R₂- is C₁-C₂₀ heteroalkylene.

44. The compound according to claim 40 wherein C₁-C₄₀ heteroalkylene comprises amide, disulfide, —O—, —S—, or sulfonamide.

45. The compound according to claim 43 wherein C₁-C₂₀ heteroalkylene comprises amide, disulfide, —O—, —S—, or sulfonamide.

46. The compound of claim 35 wherein R₃ is a polyethylene glycol moiety having the formula —(CH₂CH₂O)ₙCH₂CH₂—, wherein n=1 to 4.

47. The compound of claim 35 wherein Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

48. The compound of claim 35 wherein R₃ is —(CH₂CH₂O)₂CH₂CH₂— and Y is —NH₂.

49. The compound of claim 35 wherein R₃ is —(CH₂CH₂O)₂CH₂CH₂— and Y is —SH.

50. The compound of claim 35 wherein R₃ is —(CH₂CH₂O)₄CH₂CH₂— and Y is —C(═O)OH.

51. The compound of claim 35 wherein compound 3 has the
structure 3(a) 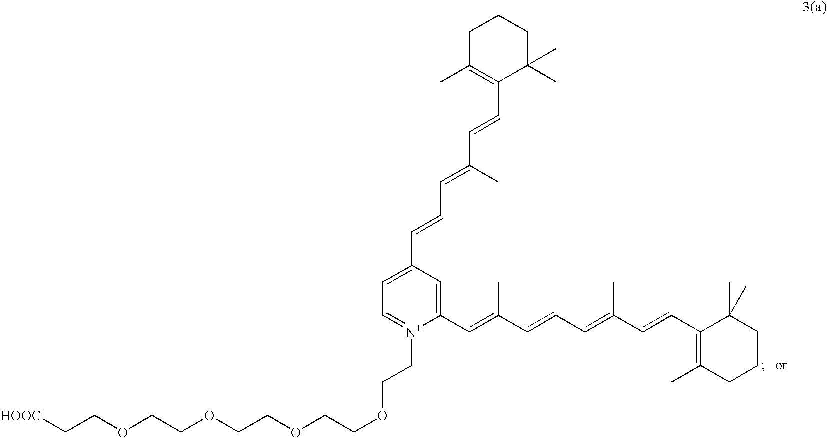
the structure 3(b) 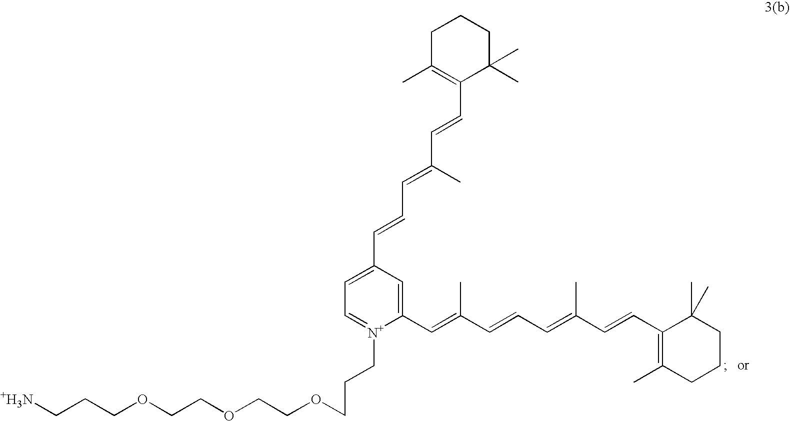
structure 3(c)

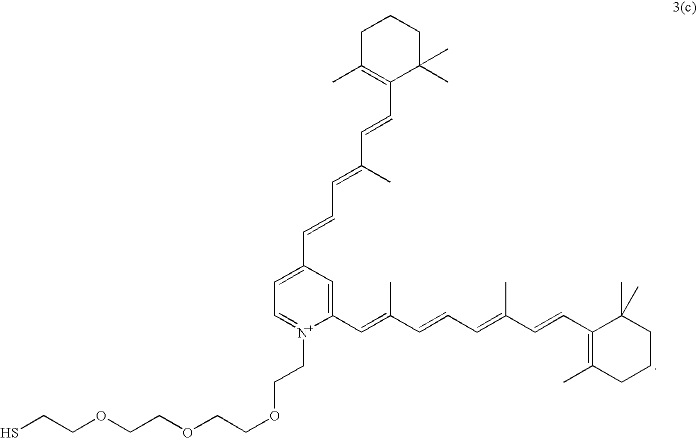

52. The compound of claim 1 wherein J is Z²-R⁰-Z³-Y, and -Z²-R⁰-Z³- has the following structure (I): 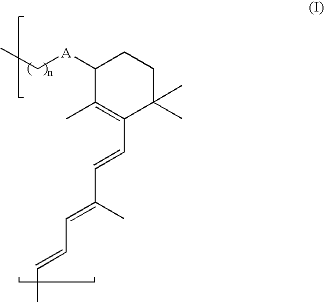
wherein n=0-12 when A is a direct bond; wherein n=1-10 when A is —O—, —NH—, —S—, —S—S—, —C(═O)NH, —NHC(═O)—, —NHC(═O)NH—, —OC(═O)—, or —C(═O)O—;
and wherein Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

53. The compound of claim 1 wherein J is Z²-R⁰-Z³-Y, and Z²-R⁰-Z³- has the following structure (II): 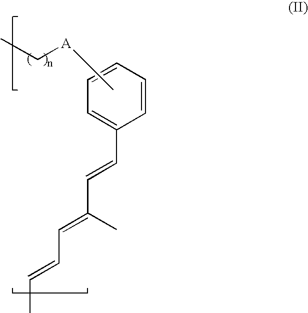
wherein n=0-12 when A is a direct bond; wherein n=1-10 when A is —O—, —NH—, —S—, —S—S—, —C(═O)NH, —NHC(═O)—, —NHC(═O)NH—, —OC(═O)—, or —C(═O)O—; and wherein Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

54. The compound of claim 1 wherein J is -Z²-Y′—R⁰, and -Z²-Y′—R⁰ has the following structure (III):

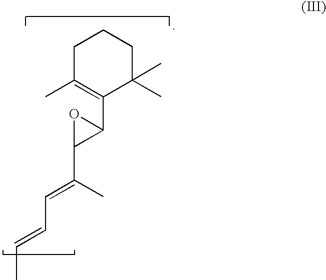

55. The compound of claim 1 wherein J is Z²-R⁰—Y, and -Z²-R⁰—Y has the following structure (IV):

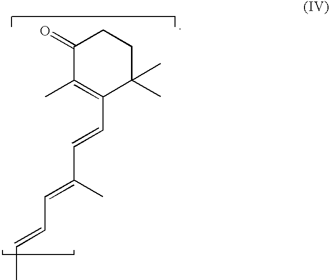

56. The compound according to claim 1 wherein compound 1 has either structure 1(a) 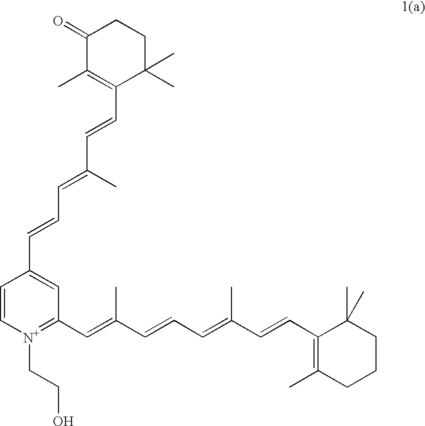
or structure 1(b)

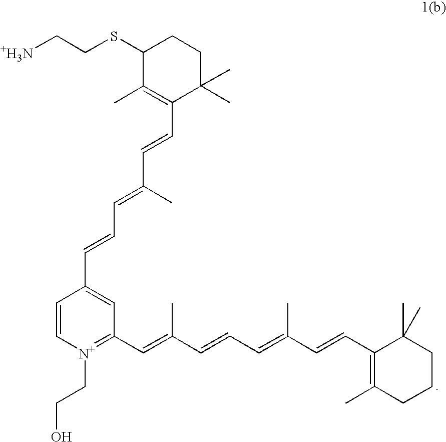

57. The compound of claim 1 wherein compound 1 has the structure 1(c):

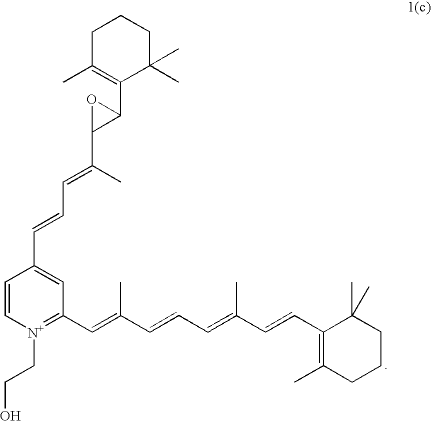

58. The compound of claim 18 wherein J is -Z²-R⁰-Z³-Y, and -Z²-R⁰-Z³ has the structure (V): 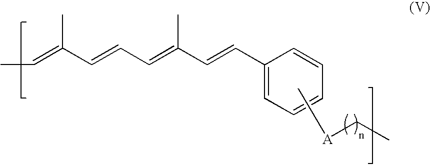
wherein n=0-12 when A is a direct bond; wherein n=1-10 when A is —O—, —NH—, —S—, —S—S—, —C(═O)NH, —NHC(═O)—, —NHC(═O)NH—, —OC(═O)—, or —C(═O)O—; and wherein Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

59. The compound of claim 18 wherein J is -Z²-R⁰-Z³-Y, and -Z²-R⁰-Z³ has the following structure (VI): 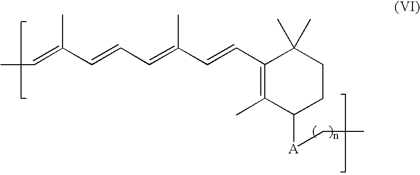
wherein n=0-12 when A is a direct bond; wherein n=1-10 when A is —O—, —NH—, —S—, —S—S—, —C(═O)NH, —NHC(═O)—, —NHC(═O)NH—, —OC(═O)—, or —C(═O)O—; and wherein Y is —OH, —SH, —NH₂, —C(═O)OH, oxo, hydrazide, N-hydroxy-succinimidyl ester, N-hydroxy-sulfosuccinimidyl ester, or pentafluorophenoxycarbonyl.

60. The compound of claim 18 wherein J is -Z²-Y′—R⁰, and -Z²-Y′—R⁰ has the following structure (VII):

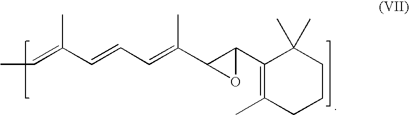

61. The compound of claim 18 wherein J is -Z²-R⁰—Y, and -Z²-R⁰—Y has the following structure (VIII):

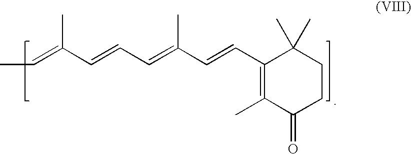

62. The compound according to claim 18 wherein compound 2 has the structure 2(a) 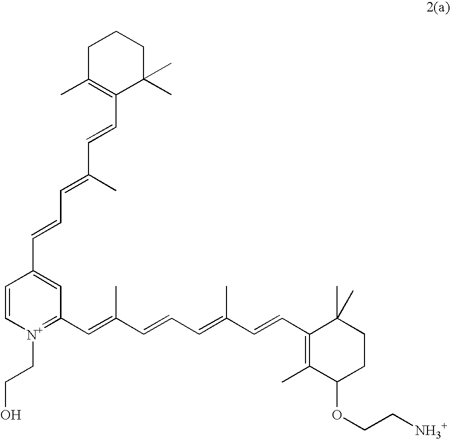
or structure 2(b)

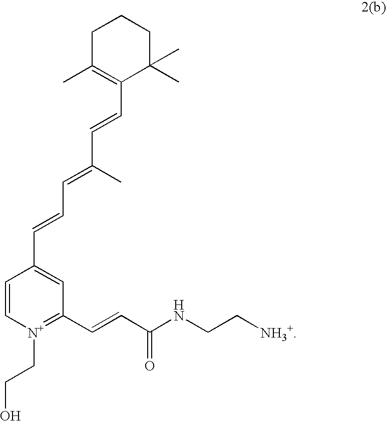

63. An immunogen comprising at least one molecule of the compound according to any one of claims 1, 18 and 35, wherein the compound is conjugated to a carrier polypeptide.

64. The immunogen of claim 63 wherein the carrier polypeptide is selected from keyhole limpet hemocyanin, bovine serum albumin, ovalbumin, tetanus toxoid, diphtheria toxoid, E. coli heat-labile enterotoxin B subunit, polyglutamate, glucose oxidase, rabbit serum albumin, sperm whale myoglobin, human thyroglobulin, and Pasteurella haemolytica leukotoxin polypeptide.

65-66. (canceled)

67. A composition comprising the immunogen according to claim 63 and a physiologically acceptable excipient.

68. The composition of claim 67 further comprising an adjuvant.

69. An isolated antibody, or an antigen-binding fragment thereof, that binds specifically to retinoid N-retinylidene-N-retinyl-ethanolamine (A2E).

70. The antibody of claim 69 wherein the antibody is a polyclonal antibody or a monoclonal antibody.

71. (canceled)

72. The antibody of claim 70 wherein the monoclonal antibody is selected from a mouse monoclonal antibody, a human monoclonal antibody, a rat monoclonal antibody, and a hamster monoclonal antibody.

73. The antibody of claim 69 wherein the antibody is a chimeric antibody or a humanized antibody.

75. The antibody of claim 69 wherein the antigen binding fragment thereof is selected from an Fab, Fab′, F(ab′)₂, Fd, and Fv.

76. (canceled)

77. The antibody of claim 69 wherein the antibody comprises a single chain antibody.

78. The antibody of claim 69 wherein the antibody is a recombinant antibody.

79. A host cell that expresses the antibody of claim 69.

80. A composition comprising an antibody, or antigen-binding fragment thereof, according to claim 69 and a physiologically acceptable excipient.

81. A method for producing an antibody that specifically binds to retinoid N-retinylidene-N-retinyl-ethanolamine (A2E) comprising administering to an animal an immunogen that comprises a compound according to any one of claims 1, 18, and 35 that is conjugated to a carrier polypeptide.

82. The method of claim 81 further comprising administering an adjuvant to the animal.

83. (canceled)

84. A method for inducing an immune response in an animal comprising administering to the animal a composition that comprises the immunogen according to claim 63.

85-86. (canceled)

87. The method according to 84 wherein the composition further comprises an adjuvant.

88. (canceled)

89. A method for detecting the presence of retinoid N-retinylidene-N-retinyl-ethanolamine (A2E) in a biological sample comprising:
(a) contacting a biological sample with an antibody, or antigen-binding fragment thereof, that specifically binds to A2E, under conditions and for a time sufficient to permit formation of an antibody/A2E complex; and
(b) detecting a level of antibody/A2E complex, and thereby detecting the presence of A2E in a sample.

90. (canceled)

91. The method of claim 89 wherein the biological sample is selected from blood, serum, vitreous fluid, aqueous humor, intraocular fluid, and tears.

92-93. (canceled)

94. The method of claim 89 wherein the antibody is detectably labeled.

95. The method of 94 wherein the antibody is detectably labeled with a fluorophore, a radionuclide, an enzyme, or biotin.

96-101. (canceled)

102. A method for treating an ophthalmic disease or disorder in a subject comprising administering to the subject a composition comprising an antibody that specifically binds to retinoid N-retinylidene-N-retinyl-ethanolamine (A2E) and a physiologically acceptable excipient.

103. The method of claim 102 wherein the ophthalmic disease or disorder is selected from macular degeneration, glaucoma, diabetic retinopathy, retinal detachment, retinal blood vessel occlusion, retinitis pigmentosa, optic neuropathy, inflammatory retinal disease, diabetic maculopathy, hemorrhagic retinopathy, retinopathy of prematurity, optic neuropathy, proliferative vitreoretinopathy, retinal dystrophy, ischemia-reperfusion related retinal injury, hereditary optic neuropathy, metabolic optic neuropathy, Stargardt's macular dystrophy, Sorsby's fundus dystrophy, Best disease, uveitis, a retinal injury, a retinal disorder associated with Parkinson's disease, a retinal disorder associated with viral infection, a retinal disorder related to light overexposure, and a retinal disorder associated with AIDS, a retinal disorder associated with Alzheimer's disease, and a retinal disorder associated with multiple sclerosis.

104-106. (canceled)

107. A method for treating an ophthalmic disease or disorder in a subject, comprising administering to the subject the immunogen according to claim 63.

108. The method according claim 107 wherein the carrier polypeptide is selected from keyhole limpet hemocyanin, bovine serum albumin, ovalbumin, tetanus toxoid, diphtheria toxoid, E. Coli heat-labile enterotoxin B subunit, polyglutamate, glucose oxidase, rabbit serum albumin, sperm whale myoglobin, human thyroglobulin, and Pasteurella haemolytica leukotoxin polypeptide.

109. (canceled)

110. The method according to 107 further comprising administering an adjuvant.

111-116. (canceled)

117. The method according to claim 107 wherein the ophthalmic disease or disorder is selected from macular degeneration, glaucoma, diabetic retinopathy, retinal detachment, retinal blood vessel occlusion, retinitis pigmentosa, optic neuropathy, inflammatory retinal disease, diabetic maculopathy, hemorrhagic retinopathy, retinopathy of prematurity, optic neuropathy, proliferative vitreoretinopathy, retinal dystrophy, ischemia-reperfusion related retinal injury, hereditary optic neuropathy, metabolic optic neuropathy, Stargardt's macular dystrophy, Sorsby's fundus dystrophy, Best disease, uveitis, a retinal injury, a retinal disorder associated with Parkinson's disease, a retinal disorder associated with viral infection, a retinal disorder related to light overexposure, and a retinal disorder associated with AIDS, a retinal disorder associated with Alzheimer's disease, and a retinal disorder associated with multiple sclerosis.

118-120. (canceled)
